(19) United States
(12) Patent Application Publication
(10) Pub. No.: US 2010/0204246 A1
(43) Pub. Date: Aug. 12, 2010

(54) 5-AMINOPYRAZOL-3-YL-3H-IMIDAZO (4,5-B) PYRIDINE DERIVATIVES AND THEIR USE FOR THE TREATMENT OF CANCER

(75) Inventors: Audrey Davies, Waltham, MA (US); Stephanos Ioannidis, Waltham, MA (US); Michelle Lamb, Waltham, MA (US); Tao Wang, Waltham, MA (US); Hai-Jun Zhang, Waltham, MA (US)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/596,218

(22) PCT Filed: Apr. 17, 2008
(86) PCT No.: PCT/GB08/01356

§ 371 Date: Mar. 29, 2010

Related U.S. Application Data
(60) Provisional application No. 60/912,676, filed on Apr. 18, 2007.
(60) Provisional application No. 60/912,511, filed on Apr. 18, 2007.

(51) Int. Cl. A61K 31/506; C07D 401/14; C07D 471/04; A61K 31/4375; A61P 35/00

(52) U.S. Cl. 514/256; 544/333; 546/118; 514/303

(57) ABSTRACT

The present invention relates to compounds of Formula (I) and to their pharmaceutical compositions, and to their methods of use. These novel compounds provide a treatment for myeloproliferative disorders and cancer.

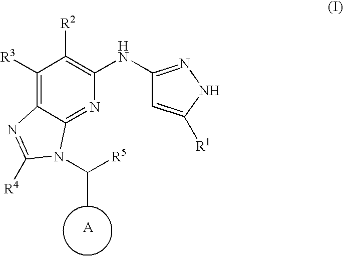

### FIELD OF THE INVENTION

The present invention relates to a novel compound, its pharmaceutical compositions and methods of use. In addition, the present invention relates to therapeutic methods for the treatment and prevention of cancers and to the use of this compound in the manufacture of medicaments for use in the treatment and prevention of myeloproliferative disorders and cancers.

### BACKGROUND OF THE INVENTION

Receptor tyrosine kinases (RTK's) are a sub-family of protein kinases that play a critical role in cell signalling and are involved in a variety of cancer related processes including cell proliferation, survival, angiogenesis and metastasis. Currently up to 100 different RTK's including tropomyosin-related kinases (Trk's) have been identified.

Trk's are the high affinity receptors activated by a group of soluble growth factors called neurotrophins (NT). The Trk receptor family has three members—TrkA, TrkB and TrkC. Among the NTs there are (i) nerve growth factor (NGF) which activates TrkA, (ii) brain-derived growth factor (BDNF) and NT-4/5 which activate TrkB and (iii) NT3 which activates TrkC. Each Trk receptor contains an extra-cellular domain (ligand binding), a trans-membrane region and an intra-cellular domain (including kinase domain). Upon binding of the ligand, the kinase catalyzes auto-phosphorylation and triggers downstream signal transduction pathways.

Trk's are widely expressed in neuronal tissue during its development where Trk's are critical for the maintenance and survival of these cells. A post-embryonic role for the Trk/neurotrophin axis (or pathway), however, remains in question. There are reports showing that Trk's play important role in both development and function of the nervous system (Patapoutian, A. et al Current Opinion in Neurobiology, 2001, 11, 272-280).

In the past decade, a considerable number of literature documentations linking Trk signalling with cancer have published. For example, while Trk's are expressed at low levels outside the nervous system in the adult, Trk expression is increased in late stage prostate cancers. Both normal prostate tissue and androgen-dependent prostate tumors express low levels of Trk A and undetectable levels of Trk B and C. However, all isoforms of Trk receptors as well as their cognate ligands are up-regulated in late stage, androgen-independent prostate cancer. There is additional evidence that these late stage prostate cancer cells become dependent on the Trk/neurotrophin axis for their survival. Therefore, Trk inhibitors may yield a class of apoptosis-inducing agents specific for androgen-independent prostate cancer (Weeraratna, A. T. et al The Prostate, 2000, 45, 140-148).

Furthermore, the literature also shows that over-expression, activation, amplification and/or mutation of Trk's are associated with secretory breast carcinoma (Cancer Cell, 2002, 2, 367-376), colorectal cancer (Bardelli et al Science, 2003, 300, 949-949) and ovarian cancer (Davidson, B. et al Clinical Cancer Research, 2003, 9, 2248-2259).

There are a few reports of selective Trk tyrosine kinase inhibitors. Cephalon described CEP-751, CEP-701 (George, D. et al Cancer Research, 1999, 59, 2395-2341) and other indolocarbazole analogues (WO0114380) as Trk inhibitors. It was shown that CEP-701 and/or CEP751, when combined with surgically or chemically induced androgen ablation, offered better efficacy compared with mono-therapy alone. GlaxoSmithKline disclosed certain oxindole compounds as Trk A inhibitors in WO0220479 and WO0220513. Recently, Japan Tobacco reported pyrazolyl condensed cyclic compounds as Trk inhibitors (JP2003231687A). Pfizer also recently published certain isothiazole Trk A inhibitors (Bioorg. Med. Chem. Lett. 2006, 16, 3444-3448).

In addition to the above, Vertex Pharmaceuticals have described pyrazole compounds as inhibitors of GSK3, Aurora, etc. in WO0250065, WO0262789, WO03027111 and WO200437814; and AstraZeneca have reported pyrazole compounds as inhibitors against IGF-1 receptor kinase (WO0348133). AstraZeneca have also reported Trk inhibitors in International Applications WO 2005/049033, WO 2005/103010, WO 2006/082392, WO 2006/087530, and WO 2006/087538.

Another such family of RTK's is the JAK family. The JAK (Janus-associated kinase)/STAT (signal transducers and activators or transcription) signalling pathway is involved in a variety of hyperproliferative and cancer related processes including cell-cycle progression, apoptosis, angiogenesis, invasion, metastasis and evasion of the immune system (Haura et al., Nature Clinical Practice Oncology, 2005, 2(6), 315-324; Verna et al., Cancer and Metastasis Reviews, 2003, 22, 423-434).

The JAK family consists of four non-receptor tyrosine kinases Tyk2, JAK1, JAK2, and JAK3, which play a critical role in cytokine- and growth factor mediated signal transduction. Cytokine and/or growth factor binding to cell-surface receptor(s), promotes receptor dimerization and facilitates activation of receptor-associated JAK by autophosphorylation. Activated JAK phosphorylates the receptor, creating docking sites for SH2 domain-containing signalling proteins, in particular the STAT family of proteins (STAT1, 2, 3, 4, 5a, 5b and 6). Receptor-bound STATs are themselves phosphorylated by JAKs, promoting their dissociation from the receptor, and subsequent dimerization and translocation to the nucleus. Once in the nucleus, the STATs bind DNA and cooperate with other transcription factors to regulate expression of a number of genes including, but not limited to, genes encoding apoptosis inhibitors (e.g. Bcl-XL, Mcl-1) and cell cycle regulators (e.g. Cyclin D1/D2, c-myc) (Haura et al., Nature Clinical Practice Oncology, 2005, 2(6), 315-324; Verna et al., Cancer and Metastasis Reviews, 2003, 22, 423-434).

Over the past decade, a considerable amount of scientific literature linking constitutive JAK and/or STAT signalling with hyperproliferative disorders and cancer has been published. Constitutive activation of the STAT family, in particular STAT3 and STATS, has been detected in a wide range of cancers and hyperproliferative disorders (Haura et al., Nature Clinical Practice Oncology, 2005, 2(6), 315-324). Furthermore, aberrant activation of the JAK/STAT pathway provides an important proliferative and/or anti-apoptotic drive downstream of many kinases (e.g. Flt3, EGFR) whose constitutive activation have been implicated as key drivers in a variety of cancers and hyperproliferative disorders (Tibes et al., Annu Rev Pharmacol Toxicol 2550, 45, 357-384; Choudhary et al., International Journal of Hematology 2005, 82(2), 93-99; Sordella et al., Science 2004, 305, 1163-1167). In addition, impairment of negative regulatory proteins, such as the suppressors of cytokine signalling (SOCS) proteins, can also influence the activation status of the JAK/STAT signalling pathway in disease (J C Tan and Rabkin R, Pediatric Nephrology 2005, 20, 567-575).

Several mutated forms of JAK2 have been identified in a variety of disease settings. For example, translocations resulting in the fusion of the JAK2 kinase domain with an oligomerization domain, TEL-JAK2, Bcr-JAK2 and PCM 1-JAK2, have been implicated in the pathogenesis of various hematologic malignancies (S D Turner and Alesander D R, Leukemia, 2006, 20, 572-582). More recently, a unique acquired mutation encoding a valine-to-phenylalanine (V617F) substitution in JAK2 was detected in a significant number of polycythemia vera, essential thrombocythemia and idiopathic myelofibrosis patients and to a lesser extent in several other diseases. The mutant JAK2 protein is able to activate downstream signalling in the absence of cytokine stimulation, resulting in autonomous growth and/or hypersensitivity to cytokines and is believed to play a critical role in driving these diseases (M J Percy and McMullin M F, Hematological Oncology 2005, 23(3-4), 91-93).

JAKs (in particular JAK3) play an important biological roles in the immunosuppressive field and there are reports of using JAK kinase inhibitors as tools to prevent organ transplant rejections (Changelian, P. S. et al, Science, 2003, 302, 875-878). Merck (Thompson, J. E. et al Bioorg. Med. Chem. Lett. 2002, 12, 1219-1223) and Incyte (WO2005/105814) reported imidazole based JAK2/3 inhibitors with enzyme potency at single nM levels. Recent Vertex PCT publications have described azaindoles as JAK inhibitors (WO2005/95400). AstraZeneca has published quinoline-3-carboxamides as JAK3 inhibitors (WO2002/92571).

In addition to the above, Vertex Pharmaceuticals has described pyrazole compounds as inhibitors of GSK3, Aurora, etc. in WO2002/50065, WO2002/62789, WO2003/027111 and WO2004/37814; and AstraZeneca has reported pyrazole compounds as inhibitors against IGF-1 receptor kinase WO2003/48133- and Trk in WO2005/049033, WO2005/103010, WO2006/082392.

### SUMMARY OF THE INVENTION

In accordance with the present invention, the applicants have hereby discovered novel compounds of Formula (I):

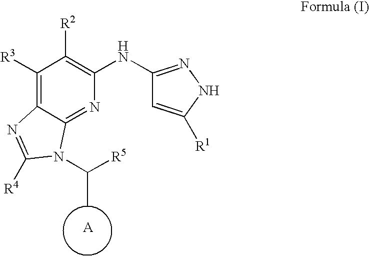

or pharmaceutically acceptable salts thereof.

The compounds of Formula (I) are believed to possess Trk kinase inhibitory activity and are accordingly useful for their anti-proliferation and/or proapoptotic (such as anti-cancer) activity and in methods of treatment of the human or animal body. The invention also relates to processes for the manufacture of said compounds, or pharmaceutically acceptable salts thereof, to pharmaceutical compositions containing them and to their use in the manufacture of medicaments for use in the production of an anti-proliferation and/or proapoptotic effect in warm-blooded animals such as man.

Also in accordance with the present invention the applicants provide methods of using such compounds, or pharmaceutically acceptable salts thereof, in the treatment of cancer.

The properties of the compounds of Formula (I) are expected to be of value in the treatment of disease states associated with cell proliferation such as cancers (solid tumors and leukemia), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

Furthermore, the compounds of Formula (I), or pharmaceutically acceptable salts thereof, are expected to be of value in the treatment or prophylaxis of cancers selected from congenital fibrosarcoma, mesoblastic nephroma, mesothelioma, acute myeloblastic leukemia, acute lymphocytic leukemia, multiple myeloma, melanoma, oesophageal cancer, myeloma, hepatocellular, pancreatic, cervical cancer, Ewings sarcoma, neuroblastoma, Kaposi sarcoma, ovarian cancer, breast cancer including secretory breast cancer, colorectal cancer, prostate cancer including hormone refractory prostate cancer, bladder cancer, melanoma, lung cancer—non small cell lung cancer (NSCLC), and small cell lung cancer (SCLC), gastric cancer, head and neck cancer, renal cancer, lymphoma, thyroid cancer including papillary thyroid cancer, mesothelioma and leukaemia; particularly ovarian cancer, breast cancer, colorectal cancer, prostate cancer and lung cancer—NSCLC and SCLC; more particularly prostate cancer; and more particularly hormone refractory prostate cancer.

The compounds of Formula (I) are also believed to possess JAK kinase inhibitory activity and are accordingly useful for their anti-proliferation and/or pro-apoptotic activity and in methods of treatment of the human or animal body. The invention also relates to processes for the manufacture of said compound, or pharmaceutically acceptable salts thereof, to pharmaceutical compositions containing it and to its use in the manufacture of medicaments for use in the production of an anti-proliferation and/or pro-apoptotic effect in warm-blooded animals such as man. Also in accordance with the present invention the applicants provide methods of using said compound, or pharmaceutically acceptable salts thereof, in the treatment of myeloproliferative disorders, myelodysplastic syndrome and cancer.

The properties of the compounds of Formula (I) are expected to be of value in the treatment of myeloproliferative disorders, myelodysplastic syndrome, and cancer by inhibiting the tyrosine kinases, particularly the JAK family and more particularly JAK2. Methods of treatment target tyrosine kinase activity, particularly the JAK family activity and more particularly JAK2 activity, which is involved in a variety of myeloproliferative disorders, myelodysplastic syndrome and cancer related processes. Thus, inhibitors of tyrosine kinases, particularly the JAK family and more particularly JAK2, are expected to be active against myeloproliferative disorders such as chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and neoplastic disease such as carcinoma of the breast, ovary, lung, colon, prostate or other tissues, as well as leukemias, myelomas and lymphomas, tumors of the central and peripheral nervous system, and other tumor types such as melanoma, fibrosarcoma and osteosarcoma. Tyrosine kinase inhibitors, particularly the JAK family inhibitors and more particularly JAK2 inhibitors are also expected to be useful for the treatment other proliferative diseases including but not limited to autoimmune, inflammatory, neurological, and cardiovascular diseases.

Furthermore, the compounds of Formula (I), or pharmaceutically acceptable salts thereof, are expected to be of value in the treatment or prophylaxis of against myeloproliferative disorders selected from chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia; particularly myeloma, leukemia, ovarian cancer, breast cancer and prostate cancer.

### DETAILED DESCRIPTION

The present invention provides compounds of Formula (I):

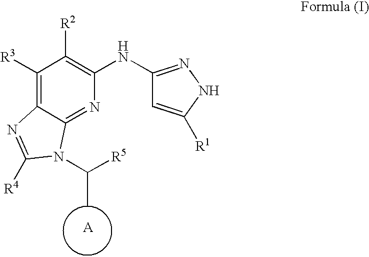

or pharmaceutically acceptable salts thereof, wherein
Ring A may be selected from heterocyclyl, wherein said heterocyclyl may be optionally substituted with one or more R⁶;
R¹ may be selected from H, —CN, C₁₋₆alkyl, C₂₋₆alkenyl, C₂₋₆alkynyl, 3- to 5-membered carbocyclyl, 5-membered heterocyclyl, —OR^{1a}, —SR^{1a}, —N(R^{1a})₂, —N(R^{1a})C(O)R^{1b}, —N(R^{1a})N(R^{1a})₂, —NO₂, —C(O)H, —C(O)R^{1b}, —C(O)₂R^{1a}, —C(O)N(R^{1a})₂, —OC(O)N(R^{1a})₂, —N(R^{1a})C(O)₂R^{1a}, —N(R^{1a})C(O)N(R^{1a})₂, —OC(O)R^{1b}, —S(O)R^{1b}, —S(O)₂R^{1b}, —S(O)₂N(R^{1a})₂, —N(R^{1a})S(O)₂R^{1b}, —C(R^{1a})═N(R^{1a}), and —C(R^{1a})═N(OR^{1a}), wherein said C₁₋₆alkyl, C₂₋₆alkenyl, C₂₋₆alkynyl, 3- to 5-membered carbocyclyl, and 5-membered heterocyclyl may be optionally substituted with one or more R¹⁰;
R^{1a} in each occurrence may be independently selected from H and C₁₋₆alkyl, 3- to 5-membered carbocyclyl, and 5-membered heterocyclyl, wherein said C₁₋₆alkyl, 3- to 5-membered carbocyclyl, and 5-membered heterocyclyl in each occurrence may be optionally and independently substituted with one or more R¹⁰;
R^{1b} in each occurrence may be independently selected from C₁₋₆alkyl, C₂₋₆alkenyl, C₂₋₆alkynyl, 3- to 5-membered carbocyclyl, and 5-membered heterocyclyl, wherein said C₁₋₆alkyl, C₂₋₆alkenyl, C₂₋₆alkynyl, 3- to 5-membered carbocyclyl, and 5-membered heterocyclyl in each occurrence may be optionally and independently substituted with one or more R¹⁰;
R² may be selected from H, halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —N(R^{2a})N(R^{2a})₂, —NO₂, —C(O)H, —C(O)R^{2b}, —C(O)₂R^{2a}, —C(O)N(R^{2a})₂, —OC(O)N(R^{2a})₂, —N(R^{2a})C(O)₂R^{2a}, —N(R^{2a})C(O)N(R^{2a})₂, —OC(O)R^{2b}, —S(O)R^{2b}, —S(O)₂R^{2b}, —S(O)₂N(R^{2a})₂, —N(R^{2a})S(O)₂R^{2b}, —C(R^{2a})═N(R^{2a}), and —C(R^{2a})═N(OR^{2a}), wherein said C₁₋₆alkyl, C₂₋₆alkenyl, C₂₋₆alkynyl, carbocyclyl, and heterocyclyl may be optionally substituted with one or more R²⁰;
R^{2a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R²⁰;
R^{2b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R²⁰;
R³ may be selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —N(R^{3a})N(R^{3a})₂, —NO₂, —C(O)H, —C(O)R^{3b}, —C(O)₂R^{3a}, —C(O)N(R^{3a})₂, —OC(O)N(R^{3a})₂, —N(R^{3a})C(O)₂R^{3a}, —N(R^{3a})C(O)N(R^{3a})₂, —OC(O)R^{3b}, —S(O)R^{3b}, —S(O)₂R^{3b}, —S(O)₂N(R^{3a})₂, —N(R^{3a})S(O)₂R^{3b}, —C(R^{3a})═N(R^{3a}), and —C(R^{3a})═N(OR^{3a}), wherein said C₁₋₆alkyl, C₂₋₆alkenyl, C₂₋₆alkynyl, carbocyclyl, and heterocyclyl may be optionally substituted with one or more R³⁰;
R^{3a} in each occurrence may be independently selected from H, carbocyclyl, and heterocyclyl, wherein said C₁₋₆alkyl, carbocyclyl, and heterocyclyl in each occurrence may be optionally and independently substituted with one or more R³⁰;
R^{3b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R³⁰;
R⁴ may be selected from H, halo, —CN, C₁₋₆alkyl, C₂₋₆alkenyl, C₂₋₆alkynyl, carbocyclyl, heterocyclyl, —OR^{4a}, —SR^{4a}, —N(R^{4a})₂, —N(R^{4a})C(O)R^{4b}, —N(R^{4a})N(R^{4a})₂, —NO₂, —C(O)H, —C(O)R^{4b}, —C(O)₂R^{4a}, —C(O)N(R^{4a})₂, —OC(O)N(R^{4a})₂, —N(R^{4a})C(O)₂R^{4a}, —N(R^{4a})C(O)N(R^{4a})₂, —OC(O)R^{4b}, —S(O)R^{4b}, —S(O)₂R^{4b}, —S(O)₂N(R^{4a})₂, —N(R^{4a})S(O)₂R^{4b}, —C(R^{4a})═N(R^{4a}), and —C(R^{4a})═N(OR^{4a}), wherein said C₁₋₆alkyl, C₂₋₆alkenyl, C₂₋₆alkynyl, carbocyclyl, and heterocyclyl may be optionally substituted with one or more R⁴⁰;
R^{4a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R⁴⁰;
R^{4b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R⁴⁰;
R⁵ may be selected from H, —CN, C₁₋₆alkyl, C₂₋₆alkenyl, C₂₋₆alkynyl, carbocyclyl, heterocyclyl, —N(R^{5a})C(O)R^{5b}, —N(R^{5a})N(R^{5a})₂, —NO₂, —C(O)H, —C(O)R^{5b}, —C(O)₂R^{5a}, —C(O)N(R^{5a})₂, —OC(O)N(R^{5a})₂, —N(R^{5a})C(O)₂R^{5a}, —N(R^{5a})C(O)N(R^{5a})₂, —OC(O)R^{5b}, —S(O)R^{5b}, —S(O)₂R^{5b}, —S(O)₂N(R^{5a})₂, —N(R^{5a})S(O)₂R^{5b}, —C(R^{5a})═N(R^{5a}), and —C(R^{5a})═N(OR^{5a}), wherein said C₁₋₆alkyl, C₂₋₆alkenyl, C₂₋₆alkynyl, carbocyclyl, and heterocyclyl may be optionally substituted with one or more R⁵⁰;
R^{5a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R⁵⁰;
R^{5b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R⁵⁰;
R⁶ in each occurrence may be independently selected from halo, —CN, C₁₋₆alkyl, C₂₋₆alkenyl, C₂₋₆alkynyl, carbocyclyl, heterocyclyl, —OR^{6a}, —SR^{6a}, —N(R^{6a})₂, —N(R^{6a})C(O)R^{6b}, —N(R^{6a})N(R^{6a})₂, —NO₂, —C(O)H, —C(O)R^{6b}, —C(O)₂R^{6a}, —C(O)N(R^{6a})₂, —OC(O)N(R^{6a})₂, —N(R^{6a})C(O)₂R^{6a}, —N(R^{6a})C(O)N(R^{6a})₂, —OC(O)R^{6b}, —S(O)R^{6b}, —S(O)₂R^{6b}, —S(O)₂N(R^{6a})₂, —N(R^{6a})S(O)₂R^{6b}, —C(R^{6a})═N(R^{6a}), and —C(R^{6a})═N(OR^{6a}), wherein said C₁₋₆alkyl, C₂₋₆alkenyl, C₂₋₆alkynyl, carbocyclyl, and heterocyclyl may be optionally substituted with one or more R⁶⁰;
R^{6a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R⁶⁰;
R^{6b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R⁶⁰;
R¹⁰ in each occurrence may be independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})C(O)R^{10b}, —N(R^{10a})N(R^{10a})₂, —NO₂, —C(O)H, —C(O)R^{10b}, —C(O)₂R^{10a}, —C(O)N(R^{10a})₂, —OC(O)N(R^{10a})₂, —N(R^{10a})C(O)₂R^{10a}, —N(R^{10a})C(O)N(R^{10a})₂, —OC(O)R^{10b}, —S(O)R^{10b}, —S(O)₂R^{10b}, —S(O)₂N(R^{10a})₂, —N(R^{10a})S(O)₂R^{10b}, —C(R^{10a})═N(R^{10a}), and —C(R^{10a})═N(OR^{10a}), wherein said C₁₋₆alkyl, C₂₋₆alkenyl, C₂₋₆alkynyl, carbocyclyl, and heterocyclyl in each occurrence may be optionally and independently substituted with one or more R^{a};
R^{10a} in each occurrence may be independently selected from H and C₁₋₆alkyl, wherein said C₁₋₆alkyl in each occurrence may be optionally and independently substituted with one or more R^{a};
R^{10b} in each occurrence may be independently selected from C₁₋₆alkyl, C₂₋₆alkenyl, and C₂₋₆alkynyl, wherein said C₁₋₆alkyl, C₂₋₆alkenyl, and C₂₋₆alkynyl in each occurrence may be optionally and independently substituted with one or more R^{a};
R²⁰ in each occurrence may be independently selected from halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, —N(R^{20a})C(O)R^{20b}, —N(R^{20a})N(R^{20a})₂, —NO₂, —C(O)H, —C(O)R^{20b}, —C(O)₂R^{20a}, —C(O)N(R^{20a})₂, —OC(O)N(R^{20a})₂, —N(R^{20a})C(O)₂R^{20a}, —N(R^{20a})C(O)N(R^{20a})₂, —OC(O)R^{20b}, —S(O)R^{20b}, —S(O)₂R^{20b}, —S(O)₂N(R^{20a})₂, —N(R^{20a})S(O)₂R^{20b}, —C(R^{20a})═N(R^{20a}), and —C(R^{20a})—N(OR^{20a}), wherein said C₁₋₆alkyl, C₂₋₆alkenyl, C₂₋₆alkynyl, carbocyclyl, and heterocyclyl in each occurrence may be optionally and independently substituted with one or more R^{b};
R^{20a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R^{b};
R^{20b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R^{b};
R³⁰ in each occurrence may be independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})C(O)R^{30b}, —N(R^{30a})N(R^{30a})₂, —NO₂, —C(O)H, —C(O)R^{30b}, —C(O)₂R^{30a}, —C(O)N(R^{30a})₂, —OC(O)N(R^{30a})₂, —N(R^{30a})C(O)₂R^{30a}, —N(R^{30a})C(O)N(R^{30a})₂, —OC(O)R^{30b}, —S(O)R^{30b}, —S(O)₂R^{30b}, —S(O)₂N(R^{30a})₂, —N(R^{30a})S(O)₂R^{30b}, —C(R^{30a})═N(R^{30a}), and —C(R^{30a})═N(OR^{30a}), wherein said C₁₋₆alkyl, C₂₋₆alkenyl, C₂₋₆alkynyl, carbocyclyl, and heterocyclyl in each occurrence may be optionally and independently substituted with one or more R^{c};
R^{30a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R^{c};
R^{30b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R^{c};
R⁴⁰ in each occurrence may be independently selected from halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})C(O)R^{40b}, —N(R^{40a})N(R^{40a})₂, —NO₂, —C(O)H, —C(O)R^{40b}, —C(O)₂R^{40a}, —C(O)N(R^{40a})₂, —OC(O)N(R^{40a})₂, —N(R^{40a})C(O)₂R^{40a}, —N(R^{40a})C(O)N(R^{40a})₂, —OC(O)R^{40b}, —S(O)R^{40b}, —S(O)₂R^{40b}, —S(O)₂N(R^{40a})₂, —N(R^{40a})S(O)₂R^{40b}, —C(R^{40a})═N(R^{40a}) and —C(R^{40a})═N(OR^{40a}), wherein said C₁₋₆alkyl, C₂₋₆alkenyl, C₂₋₆alkynyl, carbocyclyl, and heterocyclyl in each occurrence may be optionally and independently substituted with one or more R^{d};
R^{40a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R^{d};
R^{40b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R^{d};
R⁵⁰ in each occurrence may be independently selected from halo, —CN, C₁₋₆alkyl, C₂₋₆alkenyl, C₂₋₆alkynyl, carbocyclyl, heterocyclyl, —OR^{50a}, —SR^{50a}, —N(R^{50a})₂, —N(R^{50a})C(O)R^{50b}, —N(R^{50a})N(R^{50a})₂, —NO₂, —C(O)H, —C(O)R^{50b}, —C(O)₂R^{50a}, —C(O)N(R^{50a)}₂, —OC(O)N(R^{50a})₂, —N(R^{50a})C(O)₂R^{50a}, —(R^{50a})C(O)N(R^{50a})₂, —OC(O)R^{50b}, —S(O)R^{50b}, —S(O)₂R^{50b}, —S(O)₂N(R^{50a})₂, —N(R^{50a})S(O)₂R^{50b}, —C(R^{50a})═N(R^{50a}), and —C(R^{50a})═N(OR^{50a}), wherein said C₁₋₆alkyl, C₂₋₆alkenyl, C₂₋₆alkynyl, carbocyclyl, and heterocyclyl in each occurrence may be optionally and independently substituted with one or more R^{e};
R^{50a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R^{e};
R^{50b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R^{e};
R⁶⁰ in each occurrence may be independently selected from halo, —CN, C₁₋₆alkyl, C₂₋₆alkenyl, C₂₋₆alkynyl, carbocyclyl, heterocyclyl, —OR^{60a}, —SR^{60a}, —N(R^{60a})₂, —N(R^{60a})C(O)R^{60b}, —N(R^{60a})N(R^{60a})₂, —NO₂, —C(O)H, —C(O)R^{60b}, —C(O)₂R^{60a}, —C(O)N(R^{60a})₂, —OC(O)N(R^{60a})₂, —N(R^{60a})C(O)₂R^{60a}, —N(R^{60a})C(O)N(R^{60a})₂, —OC(O)R^{60b}, —S(O)R^{60b}, —S(O)₂R^{60b}, —S(O)₂N(R^{60a})₂, —N(R^{60a})S(O)₂R^{60b}, —C(R^{60a})═N(R^{60a}), and —C(R^{60a})═N(OR^{60a}), wherein said C₁₋₆alkyl, C₂₋₆alkenyl, C₂₋₆alkynyl, carbocyclyl, and heterocyclyl in each occurrence may be optionally and independently substituted with one or more R^{f};
R^{60a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R^{f};
R^{60b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R^{f};
R^{a}, R^{b}, R^{c}, R^{d}, R^{e} and R^{f} in each occurrence may be independently selected from halo, —CN, C₁₋₆alkyl, C₂₋₆alkenyl, C₂₋₆alkynyl, carbocyclyl, heterocyclyl, —OR^{m}, —SR^{m}, —N(R^{m})₂, —N(R^{m})C(O)Rⁿ, —N(R^{m})N(R^{m})₂, —NO₂, —C(O)H, —C(O)Rⁿ, —C(O)₂R^{m}, —C(O)N(R^{m})₂, —OC(O)N(R^{m})₂, —N(R^{m})C(O)₂R^{m}, —N(R^{m})C(O)N(R^{m})₂, —OC(O)Rⁿ, —S(O)Rⁿ, —S(O)₂Rⁿ, —S(O)₂N(R^{m})₂, —N(R^{m})S(O)₂Rⁿ, —C(R^{m})═N(R^{m}), and —C(R^{m})═N(OR^{m});
R^{m} in each occurrence may be independently selected from H and C₁₋₆alkyl; and
Rⁿ may be C₁₋₆alkyl.

In this specification the prefix C_{x-y} as used in terms such as C_{x-y}alkyl and the like (where x and y are integers) indicates the numerical range of carbon atoms that are present in the group; for example, C₁₋₄alkyl includes C₁alkyl (methyl), C₂alkyl (ethyl), C₃alkyl (propyl and isopropyl) and C₄alkyl (butyl, 1-methylpropyl, 2-methylpropyl, and t-butyl).

As used herein the term “alkyl” refers to both straight and branched chain saturated hydrocarbon radicals having the specified number of carbon atoms. References to individual alkyl groups such as “propyl” are specific for the straight chain version only and references to individual branched chain alkyl groups such as ‘isopropyl’ are specific for the branched chain version only.

The term “alkenyl” refers to both straight and branched chain hydrocarbon radicals having the specified number of carbon atoms and containing at least one carbon-carbon double bond. For example, “C₂₋₆alkenyl” includes, but is not limited to, groups such as C₂₋₆alkenyl, C₂₋₄alkenyl, ethenyl, 2-propenyl, 2-methyl-2-propenyl, 3-butenyl, 4-pentenyl, and 5-hexenyl.

The term “alkynyl” refers to both straight and branched chain hydrocarbon radicals having the specified number of carbon atoms and containing at least one carbon-carbon triple bond. For example, “C₂₋₆alkynyl” includes, but is not limited to, groups such as C₂₋₆alkynyl, C₂₋₄alkynyl, ethynyl, 2-propynyl, 2-methyl-2-propynyl, 3-butynyl, 4-pentynyl, and 5-hexynyl.

The term “halo” refers to fluoro, chloro, bromo, and iodo. In one aspect, the term “halo” may refer to fluoro, chloro, and bromo. In another aspect, “halo” may refer to fluoro and chloro.

The term “carbocyclyl” refers to a saturated, partially saturated, or unsaturated, mono or bicyclic carbon ring that contains 3 to 12 ring atoms, of which one or more —CH₂— groups may be optionally replaced with a corresponding number of —C(O)— groups. Illustrative examples of “carbocyclyl” include, but are not limited to, adamantyl, cyclopropyl, cyclobutyl, cyclopentyl, cyclopentenyl, cyclohexyl, cyclohexenyl, indanyl, naphthyl, oxocyclopentyl, 1-oxoindanyl, phenyl, and tetralinyl.

In one aspect, “carbocyclyl” may be “3- to 5-membered carbocyclyl.” The term “3- to 5-membered carbocyclyl” refers to a saturated or partially saturated monocyclic carbon ring containing 3 to 5 ring atoms, of which one or more —CH₂— groups may be optionally replaced with a corresponding number of —C(O)— groups. Illustrative examples of “3- to 5-membered carbocyclyl” include cyclopropyl, cyclobutyl, cyclopentyl, oxocyclopentyl, and cyclopentenyl.

In one aspect, “3- to 5-membered carbocyclyl” may refer to cyclopropyl, cyclobutyl, and cyclopentyl. In another aspect, “3- to 5-membered carbocyclyl” may refer to cyclopropyl.

The term “heterocyclyl” refers to a saturated, partially saturated, or unsaturated, mono or bicyclic ring containing 4 to 12 ring atoms of which at least one ring atom is selected from nitrogen, sulfur, and oxygen, and which may, unless otherwise specified, be carbon or nitrogen linked, and of which a —CH₂— group can optionally be replaced by a —C(O)—. Ring sulfur atoms may be optionally oxidized to form S-oxides. Ring nitrogen atoms may be optionally oxidized to form N-oxides. Illustrative examples of the term “heterocyclyl” include, but are not limited to, 1,3-benzodioxolyl, 3,5-dioxopiperidinyl, imidazolyl, indolyl, isoquinolinyl, isothiazolyl, isoxazolyl, morpholino, 2-oxa-5-azabicyclo[2.2.1]hept-5-yl oxopyrrolidinyl, 2-oxo-1,3-thiazolidinyl, piperazinyl, piperidyl, pyranyl, pyrazolyl, pyridinyl, pyrrolyl, pyrrolidinyl, pyrrolidinyl, pyrimidyl, pyrazinyl, pyrazolyl, pyridazinyl, 4-pyridonyl, quinolyl, tetrahydropyranyl, thiazolyl, thiadiazolyl, thiazolidinyl, thiomorpholino, thiophenyl, pyridinyl-N-oxide and quinolinyl-N-oxide.

In one aspect, “heterocyclyl” may be “6-membered heterocyclyl,” which refers to a saturated, partially saturated, or unsaturated, monocyclic ring containing 6 ring atoms, of which at least one ring atom is selected from nitrogen, sulfur, and oxygen, and of which a —CH₂— group may be optionally replaced by a —C(O)— group. Unless otherwise specified, “6-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “6-membered heterocyclyl” include, but are not limited to, morpholino, piperazinyl, piperidinyl, pyrazinyl, pyridazinyl, pyridinyl, and pyrimidinyl.

In another aspect, “heterocyclyl” may be “5-membered heterocyclyl,” which refers to a saturated, partially saturated, or unsaturated, monocyclic ring containing 5 ring atoms, of which at least one ring atom is selected from nitrogen, sulfur, and oxygen, and of which a —CH₂— group may be optionally replaced by a —C(O)— group. Unless otherwise specified, “5-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “5-membered heterocyclyl” include, but are not limited to, furanyl, imidazolyl, oxopyrrolidinyl, pyrrolyl, pyrrolidinyl, tetrahydrofuranyl, and thiazolyl.

In still another aspect, “heterocyclyl” may be “6-membered heteroaryl.” The term “6-membered heteroaryl” is intended to refer to a monocyclic, aromatic heterocyclyl ring containing 6 ring atoms. Illustrative examples of the term “6-membered heteroaryl” include, but are not limited to, pyrazinyl, pyridazinyl, pyrimidinyl, and pyridinyl. In one aspect, “6-membered heteroaryl” may refer to pyridinyl and pyrimidinyl.

Where a particular R group (e.g. R^{1a}, R¹⁰, etc.) is present in a compound of Formula (I) more than once, it is intended that each selection for that R group is independent at each occurrence of any selection at any other occurrence. For example, the —N(R)₂ group is intended to encompass: 1) those —N(R)₂ groups in which both R substituents are the same, such as those in which both R substituents are, for example, C₁₋₆alkyl; and 2) those —N(R)₂ groups in which each R substituent is different, such as those in which one R substituent is, for example, H, and the other R substituent is, for example, carbocyclyl.

Unless specifically stated, the bonding atom of a group may be any suitable atom of that group; for example, propyl includes prop-1-yl and prop-2-yl.

The phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician.

In particular, an effective amount of a compound of Formula (I) for use in the treatment of cancer is an amount sufficient to symptomatically relieve in a warm-blooded animal such as man, the symptoms of cancer, to slow the progression of cancer, or to reduce in patients with symptoms of cancer the risk of getting worse.

The term “leaving group” is intended to refer to groups readily displaceable by a nucleophile such as an amine nucleophile, and alcohol nucleophile, or a thiol nucleophile. Examples of suitable leaving groups include halo, such as chloro and bromo, and sulfonyloxy groups, such as methanesulfonyloxy, trifluoromethanesulfonate, and toluene-4-sulfonyloxy.

The term “optionally substituted,” indicates that substitution is optional and therefore it is possible for the designated group to be either substituted or unsubstituted. In the event a substitution is desired, any number of hydrogens on the designated group may be replaced with a selection from the indicated substituents, provided that the normal valency of the atoms on a particular substituent is not exceeded, and that the substitution results in a stable compound.

In one aspect, when a particular group is designated as being optionally substituted with “one or more” substituents, the particular may be unsubstituted. In another aspect, the particular group may bear one substituent. In another aspect, the particular substituent may bear two substituents. In still another aspect, the particular group may bear three substituents. In yet another aspect, the particular group may bear four substituents. In a further aspect, the particular group may bear one or two substituents. In still a further aspect, the particular group may be unsubstituted, or may bear one or two substituents.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “protecting group” is intended to refer to those groups used to prevent selected reactive groups (such as carboxy, amino, hydroxy, and mercapto groups) from undergoing undesired reactions.

Illustrative examples of suitable protecting groups for a hydroxy group include, but are not limited to, an acyl group; alkanoyl groups such as acetyl; aroyl groups, such as benzoyl; silyl groups, such as trimethylsilyl; and arylmethyl groups, such as benzyl. The deprotection conditions for the above hydroxy protecting groups will necessarily vary with the choice of protecting group. Thus, for example, an acyl group such as an alkanoyl or an aroyl group may be removed, for example, by hydrolysis with a suitable base such as an alkali metal hydroxide (for example, lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

Illustrative examples of suitable protecting groups for an amino group include, but are not limited to, acyl groups; alkanoyl groups such as acetyl; alkoxycarbonyl groups, such as methoxycarbonyl, ethoxycarbonyl, and t-butoxycarbonyl; arylmethoxycarbonyl groups, such as benzyloxycarbonyl; and aroyl groups, such benzoyl. The deprotection conditions for the above amino protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide (for example, lithium or sodium hydroxide). Alternatively an acyl group such as a t-butoxycarbonyl group may be removed, for example, by treatment with a suitable acid as hydrochloric, sulfuric, phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron trichloride). A suitable alternative protecting group for a primary amino group is, for example, a phthaloyl group, which may be removed by treatment with an alkylamine (For example, dimethylaminopropylamine or 2-hydroxyethylamine), or with hydrazine. Another suitable protecting group for an amine is, for example, a cyclic ether such as tetrahydrofuran, which may be removed by treatment with a suitable acid such as trifluoroacetic acid.

The protecting groups may be removed at any convenient stage in the synthesis using conventional techniques well known in the chemical art, or they may be removed during a later reaction step or work-up.

With reference to substituent R¹ for illustrative purposes, the following substituent definitions have the indicated meanings:

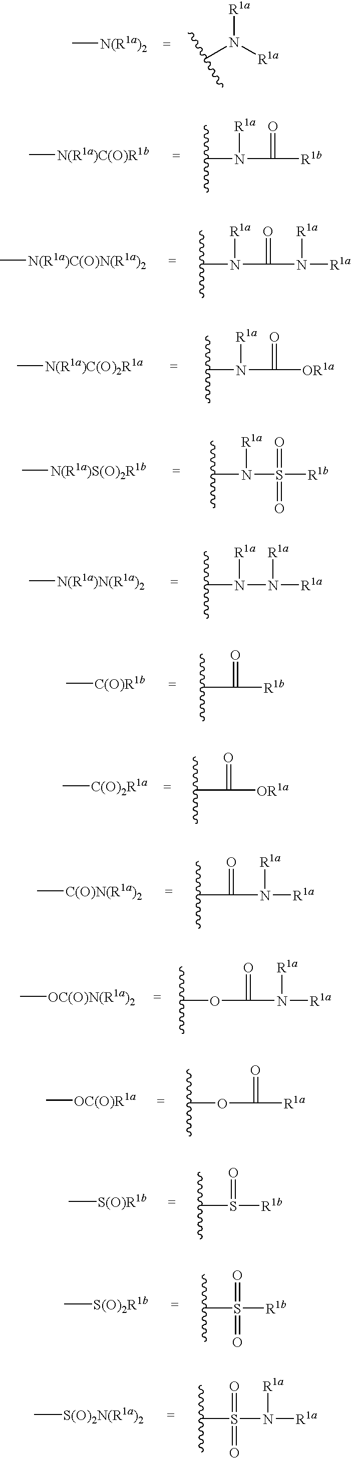

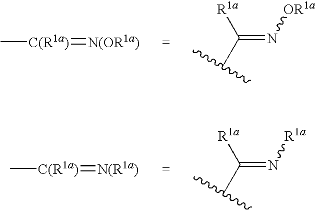

The compounds discussed herein in many instances were named and/or checked with ACD/Name by ACD/Labs®.

Compounds of Formula (I) may form stable pharmaceutically acceptable acid or base salts, and in such cases administration of a compound as a salt may be appropriate. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Examples of base salts include ammonium salts; alkali metal salts such as sodium, lithium and potassium salts; alkaline earth metal salts such as aluminum, calcium and magnesium salts; salts with organic bases such as dicyclohexylamine salts and N-methyl-D-glucamine; and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates such as dimethyl, diethyl, dibutyl; diamyl sulfates; long chain halides such as decyl, lauryl, myristyl and stearyl halides; arylalkyl halides such as benzyl bromide and others. Non-toxic physiologically-acceptable salts are preferred, although other salts may be useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

Some compounds of Formula (I) may have chiral centers and/or geometric isomeric centers (E- and Z-isomers), and it is to be understood that the invention encompasses all such optical, diastereoisomers and geometric isomers. The invention further relates to any and all tautomeric forms of the compounds of Formula (I).

It is also to be understood that certain compounds of Formula (I) can exist in solvated as well as unsolvated forms such as, for example, hydrated forms. It is to be understood that the invention encompasses all such solvated forms.

Additional embodiments of the invention are as follows. These additional embodiments relate to compounds of Formula (I) and pharmaceutically acceptable salts thereof. Such specific substituents may be used, where appropriate, with any of the definitions, claims or embodiments defined hereinbefore or hereinafter.

### Ring A

In one aspect, Ring A may be selected from 6-membered heterocyclyl, wherein said 6-membered heterocyclyl may be optionally substituted with one or more R⁶;

R⁶ in each occurrence may be independently selected from halo, —CN, C₁₋₆alkyl, C₂₋₆alkenyl, C₂₋₆alkynyl, carbocyclyl, and heterocyclyl, —OR^{6a}, —SR^{6a}, —N(R^{6a})₂, —N(R^{6a})C(O)R^{6b}, —NO₂, —C(O)H, —C(O)R^{6b}, —C(O)₂R^{6a}, —C(O)N(R^{6a})₂, —OC(O)R^{6a}, —N(R^{6a})C(O)N(R^{6a})₂, —S(O)R^{6b}, —S(O)₂R^{6b}, —S(O)₂N(R^{6a})₂, and —N(R^{6a})S(O)₂R^{6b}, wherein said C₁₋₆alkyl, C₂₋₆alkenyl, C₂₋₆alkynyl, carbocyclyl, and heterocyclyl may be optionally substituted with one or more R⁶⁰;
R^{6a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R⁶⁰;
R^{6b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R⁶⁰;
R⁶⁰ in each occurrence may be independently selected from halo, —CN, C₁₋₆alkyl, C₂₋₆alkenyl, C₂₋₆alkynyl, carbocyclyl, and heterocyclyl, —OR^{60a}, —SR^{60a}, —N(R^{60a})₂, —N(R^{60a})C(O)R^{60b}, —NO₂, —C(O)H, —C(O)R^{60b}, —C(O)₂R^{60a}, —C(O)N(R^{60a}), —OC(O)R^{60a}, —N(R^{60a})C(O)N(R^{60a})₂, —S(O)R^{60b}, —S(O)₂R^{60b}, —S(O)₂N(R^{60a})₂, and —N(R^{60a})S(O)₂R^{60b};
R^{60a} in each occurrence may be independently selected from H, carbocyclyl, and heterocyclyl; and
R^{60b} in each occurrence may be independently selected from C₁₋₆alkyl, C₂₋₆alkenyl, C₂₋₆alkynyl, carbocyclyl, and heterocyclyl.

In another aspect, Ring A may be selected from 6-membered heterocyclyl, wherein said 6-membered heterocyclyl may be optionally substituted with one or more R⁶;

R⁶ in each occurrence may be independently selected from halo, —CN, C₁₋₆alkyl, C₂₋₆alkenyl, C₂₋₆alkynyl, carbocyclyl, and heterocyclyl, —OR^{6a}, —SR^{6a}, —N(R^{6a})₂, —N(R^{6a})C(O)R^{6b}, —NO₂, —C(O)H, —C(O)R^{6b}, —C(O)₂R^{6a}, —C(O)N(R^{6a})₂, —OC(O)R^{6a}, —N(R^{6a})C(O)N(R^{6a})₂, —S(O)R^{6b}, —S(O)₂R^{6b}, —S(O)₂N(R^{6a})₂, and —N(R^{6a})S(O)₂R^{6b};
R^{6a} in each occurrence may be independently selected from H, C₁₋₆alkyl, carbocyclyl, and heterocyclyl;
R^{6b} in each occurrence may be independently selected from C₁₋₆alkyl, C₂₋₆alkenyl, C₂₋₆alkynyl, carbocyclyl, and heterocyclyl.

In still another aspect, Ring A may be selected from 6-membered heterocyclyl, wherein said 6-membered heterocyclyl may be optionally substituted with one or more R⁶;

R⁶ in each occurrence may be independently selected from halo, —CN, C₁₋₆alkyl, C₂₋₆alkenyl, and C₂₋₆alkynyl, —OR^{6a}, —SR^{6a}, —N(R^{6a})₂, —N(R^{6a})C(O)R^{6b}, —NO₂, —C(O)H, —C(O)R^{6b}, —C(O)₂R^{6a}, —C(O)N(R⁶)₂, —OC(O)R^{6a}, —N(R^{6a})C(O)N(R^{6a})₂, —S(O)R^{6b}, —S(O)₂R^{6b}, —S(O)₂N(R^{6a})₂, and —N(R^{6a})S(O)₂R^{6b};
R^{6a} in each occurrence may be independently selected from H and C₁₋₆alkyl; and
R^{6b} in each occurrence may be independently selected from C₁₋₆alkyl, C₂₋₆alkenyl, and C₂₋₆alkynyl.

In yet another aspect, Ring A may be selected from 6-membered heterocyclyl, wherein said 6-membered heterocyclyl may be optionally substituted with one or more R⁶;

R⁶ in each occurrence may be independently selected from halo, —CN, —OR^{6a}, —SR^{6a}, and —N(R^{6a});
R^{6a} in each occurrence may be independently selected from H and C₁₋₆alkyl.

In a further aspect, Ring A may be selected from 6-membered heteroaryl, wherein said 6-membered heteroaryl may be optionally substituted with one or more R⁶;

R⁶ in each occurrence may be independently selected from halo, —CN, C₁₋₆alkyl, C₂₋₆alkenyl, and C₂₋₆alkynyl, —OR^{6a}, —SR^{6a}, —N(R^{6a})₂, —N(R^{6a})C(O)R^{6b}, —NO₂, —C(O)H, —C(O)R^{6b}, —C(O)₂R^{6a}, —C(O)N(R^{6a})₂, —OC(O)R^{6a}, —N(R^{6a})C(O)N(R^{6a})₂, —S(O)R^{6b}, —S(O)₂R^{6b}, —S(O)₂N(R^{6a})₂, and —N(R^{6a})S(O)₂R^{6b};
R^{6a} in each occurrence may be independently selected from H and C₁₋₆alkyl; and
R^{6b} in each occurrence may be independently selected from C₁₋₆alkyl, C₂₋₆alkenyl, and C₂₋₆alkynyl.

In still a further aspect, Ring A may be selected from 6-membered heteroaryl, wherein said 6-membered heteroaryl may be optionally substituted with one or more R⁶;

R⁶ in each occurrence may be independently selected from halo, —CN, —OR^{6a}, —SR^{6a}, and —N(R^{6a}),
R^{6a} in each occurrence may be independently selected from H and C₁₋₆alkyl.

In yet a further aspect, Ring A may be selected from 6-membered heteroaryl, wherein said 6-membered heteroaryl may be optionally substituted with one or more R⁶; and

R⁶ may be halo.

In one aspect, Ring A may be selected from pyridinyl and pyrimidinyl, wherein said pyridinyl and pyrimidinyl may be optionally substituted with one or more R⁶; and

R⁶ in each occurrence may be independently selected from halo, —CN, and —OR^{6a}; and
R^{6a} in each occurrence may be independently selected from H and C₁₋₆alkyl.

In another aspect, Ring A may be selected from pyridinyl and pyrimidinyl, wherein said pyridinyl and pyrimidinyl may be optionally substituted with one or more R⁶; and

R⁶ may be fluoro.

In still another aspect, Ring A may be selected from pyridinyl, wherein said pyridinyl may be optionally substituted with one or more R⁶; and

R⁶ may be halo.

In yet another aspect, Ring A may be selected from pyrimidinyl, wherein said pyrimidinyl may be optionally substituted with one or more R⁶; and

R⁶ may be halo.

In a further aspect, Ring A may be selected from 5-fluoropyridin-2-yl, 3,5-difluoropyridin-2-yl, and 5-fluoropyrimidin-2-yl.

In still a further aspect, Ring A may be 3,5-difluoropyridin-2-yl.

In yet a further aspect, Ring A may be 5-fluoropyridin-2-yl.

In one aspect, Ring A may be 5-fluoropyrimidin-2-yl.

### R¹

In one aspect, R¹ may be selected from —CN, C₁₋₆alkyl, C₂₋₆alkenyl, C₂₋₆alkynyl, 3- to 5-membered carbocyclyl, 5-membered heterocyclyl, —OR^{1a}, —SR^{1a}, —N(R^{1a})₂, —N(R^{1a})C(O)R^{1b}, —NO₂, —C(O)H, —C(O)R^{1b}, —C(O)₂R^{1a}, —C(O)N(R^{1a})₂, —OC(O)R^{1b}, —N(R^{1a})C(O)N(R^{1a})₂, —S(O)R^{1b}, —S(O)₂R^{1b}, —S(O)₂N(R^{1a})₂, and —N(R^{1a})S(O)₂R^{1b}, wherein said C₁₋₆alkyl, C₂₋₆alkenyl, C₂₋₆alkynyl, 3- to 5-membered carbocyclyl, and 5-membered heterocyclyl may be optionally substituted with one or more R¹⁰;

R^{1a} in each occurrence may be independently selected from H, C₁₋₆alkyl, 3- to 5-membered carbocyclyl, and 5-membered heterocyclyl, wherein said C₁₋₆alkyl, 3- to 5-membered carbocyclyl, and 5-membered heterocyclyl in each occurrence may be optionally and independently substituted with one or more R¹⁰;
R^{1b} in each occurrence may be independently selected from C₁₋₆alkyl, C₂₋₆alkenyl, C₂₋₆alkynyl, 3- to 5-membered carbocyclyl, and 5-membered heterocyclyl wherein said C₁₋₆alkyl, C₂₋₆alkenyl, C₂₋₆alkynyl, 3- to 5-membered carbocyclyl, and 5-membered heterocyclyl in each occurrence may be optionally and independently substituted with one or more R¹⁰;
R¹⁰ in each occurrence may be independently selected from halo, —CN, C₁₋₆alkyl, C₂₋₆alkynyl, —OR^{10a}, —SR^{10a}, —N(R^{10a})₂, —N(R^{10a})C(O)R^{10b}, —NO₂, —C(O)H, —C(O)R^{10b}, —C(O)₂R^{10a}, —C(O)N(R^{10a})₂, —OC(O)R^{10b}, —(R^{10a})C(O)N(R^{10a})₂, —S(O)R^{10b}, —S(O)₂R^{10b}, —S(O)₂N(R^{10a})₂, and —N(R^{10a})S(O)₂R^{10b};
R^{10a} in each occurrence may be independently selected from H and C₁₋₆alkyl; and
R^{10b} in each occurrence may be independently selected from C₁₋₆alkyl, C₂₋₆alkenyl, and C₂₋₆alkynyl.

In another aspect, R¹ may be selected from —CN, C₁₋₆alkyl, C₂₋₆alkenyl, C₂₋₆alkynyl, 3- to 5-membered carbocyclyl, 5-membered heterocyclyl, —OR^{1a}, —SR^{1a}, —N(R^{1a})₂, —N(R^{1a})C(O)R^{1b}, —NO₂, —C(O)H, —C(O)R^{1b}, —C(O)₂R^{1a}, —C(O)N(R^{1a})₂, —OC(O)R^{1b}, —N(R^{1a})C(O)N(R^{1a})₂, —S(O)R^{1b}, —S(O)₂R^{1b}, —S(O)₂N(R^{1a})₂, and —N(R^{1a})S(O)₂R^{1b};

R^{1a} in each occurrence may be independently selected from H, C₁₋₆alkyl, 3- to 5-membered carbocyclyl, and 5-membered heterocyclyl; and

R^{1b} in each occurrence may be independently selected from C₁₋₆alkyl, C₂₋₆alkenyl, C₂₋₆alkynyl, 3- to 5-membered carbocyclyl, and 5-membered heterocyclyl.

In still another aspect, R¹ may be selected from —CN, C₁₋₆alkyl, C₂₋₆alkenyl, C₂₋₆alkynyl, 3- to 5-membered carbocyclyl, 5-membered heterocyclyl, —OR^{1a}, —SR^{1a}, —N(R^{1a})₂, —N(R^{1a})C(O)R^{1b}, —NO₂, —C(O)H, —C(O)R^{1b}, —C(O)₂R^{1a}, —C(O)N(R^{1a})₂, —OC(O)R^{1b}, —N(R^{1a})C(O)N(R^{1a})₂, —S(O)R^{1b}, —S(O)₂R^{1b}, —S(O)₂N(R^{1a})₂, and —N(R^{1a})S(O)₂R^{1b};

R^{1a} in each occurrence may be independently selected from H and C₁₋₆alkyl; and
R^{1b} in each occurrence may be independently selected from C₁₋₆alkyl, C₂₋₆alkenyl, and C₂₋₆alkynyl.

In yet another aspect, R¹ may be selected from —CN, C₁₋₆alkyl, C₂₋₆alkenyl, C₂₋₆alkynyl, 3- to 5-membered carbocyclyl, 5-membered heterocyclyl, —OR^{1a}, and —N(R^{1a})₂; and

R^{1a} in each occurrence may be independently selected from H, C₁₋₆alkyl, 3- to 5-membered carbocyclyl, and 5-membered heterocyclyl.

In a further aspect, R¹ may be selected from —CN, C₁₋₆alkyl, 3- to 5-membered carbocyclyl, 5-membered heterocyclyl, —OR^{1a}, and —N(R^{1a})₂; and

R^{1a} in each occurrence may be independently selected from H and C₁₋₆alkyl.

In still a further aspect, R¹ may be selected from —CN, C₁₋₆alkyl, 3- to 5-membered carbocyclyl, —OR^{1a}, and —N(R^{1a})₂; and

R^{1a} in each occurrence may be independently selected from H and C₁₋₆alkyl.

In yet a further aspect, R¹ may be selected from C₁₋₆alkyl, —OR^{1a}, and 3- to 5-membered carbocyclyl; and

R^{1a} may be C₁₋₆alkyl.

In one aspect, R¹ may be selected from C₁₋₆alkyl, —OR^{1a}, cyclopropyl; and R^{1a} may be C₁₋₆alkyl.

In another aspect, R¹ may be selected from methyl, cyclopropyl, methoxy, ethoxy, and isopropoxy.

In still another aspect, R¹ may be methyl.

In yet another aspect, R¹ may be cyclopropyl.

In a further aspect, R¹ may be selected from methoxy, ethoxy, and isopropoxy.

### R₂

In one aspect, R² may be selected from H, halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —NO₂, —C(O)H, —C(O)R^{2b}, —C(O)₂R^{2a}, —C(O)N(R^{2a})₂, —OC(O)R^{2a}, —N(R^{2a})C(O)N(R^{2a})₂, —S(O)R^{2b}, —S(O)₂R^{2b}, —S(O)₂N(R^{2a})₂, and —N(R^{2a})S(O)₂R^{2b}, wherein said C₁₋₆alkyl, C₂₋₆alkenyl, and C₂₋₆alkynyl, carbocyclyl, and heterocyclyl may be optionally substituted with one or more R²⁰;

R^{2a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R²⁰;
R^{2b} in each occurrence may be independently selected from C₁₋₆alkyl, C₂₋₆alkenyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R²⁰;
R²⁰ in each occurrence may be independently selected from halo, —CN, —OR^{20a}, —SR^{20a}, —N(R^{20a})₂, —N(R^{20a})C(O)R^{20b}, —NO₂, —C(O)H, —C(O)R^{20b}, —C(O)₂R^{20a}, —C(O)N(R^{20a})₂, —OC(O)R^{20a}, —N(R^{20a})C(O)N(R^{20a})₂, —S(O)R^{20b}, —S(O)₂R^{20b}, —S(O)₂N(R^{20a})₂, and —N(R^{20a})S(O)₂R^{20b};
R^{20a} in each occurrence may be independently selected from H, C₁₋₆alkyl, carbocyclyl, and heterocyclyl; and
R^{20b} in each occurrence may be independently selected from C₁₋₆alkyl, C₂₋₆alkenyl, C₂₋₆alkynyl, carbocyclyl, and heterocyclyl.

In another aspect, R² may be selected from H, halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —NO₂, —C(O)H, —C(O)R^{2b}, —C(O)₂R^{2a}, —C(O)N(R^{2a})₂, —OC(O)R^{2a}, —N(R^{2a})C(O)N(R^{2a})₂, —S(O)R^{2b}, —S(O)₂R^{2b}, —S(O)₂N(R^{2a})₂, and —N(R^{2a})S(O)₂R^{2b};

R^{2a} in each occurrence is independently selected from H, C₁₋₆alkyl, carbocyclyl, and heterocyclyl; and
R^{2b} is independently selected from C₁₋₆alkyl, C₂₋₆alkenyl, C₂₋₆alkynyl, carbocyclyl, and heterocyclyl.

In still another aspect, R² may be selected from H, halo, and C₁₋₆alkyl.

In yet another aspect, R² may be selected from H and halo.

In a further aspect, R² may be selected from H, halo, and methyl.

In still a further aspect, R² may be selected from H, fluoro, chloro, and methyl.

In yet a further aspect, R² may be selected from H and fluoro.

In one aspect, R² may be H.

In another aspect, R² may be halo.

In still another aspect, R² may be fluoro.

### R³

In one aspect, R³ may be selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —NO₂, —C(O)H, —C(O)R^{3b}, —C(O)₂R^{3a}, —C(O)N(R^{3a})₂, —OC(O)R^{2a}, —N(R^{3a})C(O)N(R^{3a})₂, —S(O)R^{3b}, —S(O)₂R^{3b}, —S(O)₂N(R^{3a})₂, and —N(R^{3a})S(O)₂R^{3b}, wherein said C₁₋₆alkyl, C₂₋₆alkenyl, and C₂₋₆alkynyl, carbocyclyl, and heterocyclyl may be optionally substituted with one or more R³⁰;

R^{3a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R³⁰;
R^{3b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R³⁰;
R³⁰ in each occurrence may be independently selected from halo, —CN, —OR^{30a}, —SR^{30a}, —N(R^{30a})₂, —N(R^{30a})C(O)R^{30b}, —NO₂, —C(O)H, —C(O)R^{30b}, —C(O)₂R^{30a}, —C(O)N(R^{30a})₂, —OC(O)R^{30a}, —N(R^{30a})C(O)N(R^{30a})₂, —S(O)R^{30b}, —S(O)₂R^{30b}, —S(O)₂N(R^{30a})₂, and —N(R^{30a})S(O)₂R^{30b};
R^{30a} in each occurrence may be independently selected from H, C₁₋₆alkyl, carbocyclyl, and heterocyclyl; and
R^{30b} in each occurrence may be independently selected from C₁₋₆alkyl, C₂₋₆alkenyl, carbocyclyl, and heterocyclyl.

In another aspect, R³ may be selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —NO₂, —C(O)H, —C(O)R^{3b}, —C(O)₂R^{3a}, —C(O)N(R^{3a})₂, —OC(O)R^{3a}, —N(R^{3a})C(O)N(R^{3a})₂, —S(O)R^{3b}, —S(O)₂R^{3b}, —S(O)₂N(R^{3a})₂, and —N(R^{3a})S(O)₂R^{3b};

R^{3a} in each occurrence is independently selected from H, carbocyclyl, and heterocyclyl; and
R^{3b} is independently selected from C₁₋₆alkyl, C₂₋₆alkenyl, C₂₋₆alkynyl, carbocyclyl, and heterocyclyl.

In still another aspect, R³ may be H.

### R⁴

In one aspect, R⁴ may be selected from H, C₁₋₆alkyl, and —OR^{4a}; and

R^{4a} may be selected from H and C₁₋₆alkyl.

In another aspect, R⁴ may be selected from H, C₁₋₆alkyl, and hydroxy.

In still another aspect, R⁴ may be selected from H, methyl, and hydroxy.

In yet another aspect, R⁴ may be H.

In a further aspect, R⁴ may be methyl.

In still a further aspect, R⁴ may be hydroxy.

### R⁵

In one aspect, R⁵ may be selected from H, —CN, C₁₋₆alkyl, C₂₋₆alkenyl, and C₂₋₆alkynyl, —N(R^{5a})C(O)R^{5b}, —NO₂, —C(O)H, —C(O)R^{5b}, —C(O)₂R^{5a}, —C(O)N(R^{5a})₂, —OC(O)N(R⁵)₂, —N(R^{5a})C(O)₂R^{5a}, —N(R^{5a})C(O)N(R^{5a})₂, —OC(O)R^{5b}, —S(O)R^{5b}, —S(O)₂R^{5b}, —S(O)₂N(R^{5a})₂, and —N(R^{5a})S(O)₂R^{5b}, wherein said C₁₋₆alkyl, C₂₋₆alkenyl, and C₂₋₆alkynyl may be optionally substituted with one or more R⁵⁰;

R^{5a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R⁵⁰;
R^{5b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R⁵⁰;
R⁵⁰ in each occurrence may be independently selected from halo, —CN, carbocyclyl, heterocyclyl, —OR^{50a}, —SR^{50a}, —N(R^{50a})₂, —N(R^{50a})C(O)R^{50b}, —NO₂, —C(O)H, —C(O)R^{50b}, —C(O)₂R^{50a}, —C(O)N(R^{50a})₂, —OC(O)R^{50a}, —N(R^{50a})C(O)N(R^{50a})₂, —S(O)R^{50b}, —S(O)₂R^{50b}, —S(O)₂N(R^{50a})₂, and —N(R^{50a})S(O)₂R^{50b};
R^{50a} in each occurrence may be independently selected from H, C₁₋₆alkyl, carbocyclyl, and heterocyclyl; and
R^{50b} in each occurrence may be independently selected from C₁₋₆alkyl, C₂₋₆alkenyl, C₂₋₆alkynyl, carbocyclyl, and heterocyclyl.

In another aspect, R⁵ may be selected from H, C₁₋₆alkyl, C₂₋₆alkenyl, C₂₋₆alkynyl, wherein said C₁₋₆alkyl, C₂₋₆alkenyl, and C₂₋₆alkynyl may be optionally substituted with one or more R⁵⁰;

R⁵⁰ in each occurrence may be independently selected from halo, —CN, carbocyclyl, heterocyclyl, —OR^{50a}, —SR^{50a}, —N(R^{50a})₂, —N(R^{50a})C(O)R^{50b}, —NO₂, —C(O)H, —C(O)R^{50b}, —C(O)₂R^{50a}, —C(O)N(R^{50a})₂, —OC(O)R^{50a}, —N(R^{50a})C(O)N(R^{50a})₂, —S(O)R^{50b}, —S(O)₂R^{50b}, —S(O)₂N(R^{50a})₂, and —N(R^{50a})S(O)₂R^{50b};
R^{50a} in each occurrence may be independently selected from H, C₁₋₆alkyl, carbocyclyl, and heterocyclyl;
R^{50b} in each occurrence may be independently selected from C₁₋₆alkyl, C₂₋₆alkenyl, C₂₋₆alkynyl, carbocyclyl, and heterocyclyl.

In still another aspect, R⁵ may be selected from H, C₁₋₆alkyl, C₂₋₆alkenyl, C₂₋₆alkynyl, wherein said C₁₋₆alkyl, C₂₋₆alkenyl, and C₂₋₆alkynyl may be optionally substituted with one or more R⁵⁰;

R⁵⁰ in each occurrence may be independently selected from halo, —CN, carbocyclyl, heterocyclyl, —OR^{50a}, —SR^{50a}, —N(R^{50a})₂, —N(R^{50a})C(O)R^{50b}, —NO₂, —C(O)H, —C(O)R^{50b}, —C(O)₂R^{50a}, —C(O)N(R^{50a})₂, —OC(O)R^{50a}, —N(R^{50a})C(O)N(R^{50a})₂, —S(O)R^{50b}, —S(O)₂R^{50b}, —S(O)₂N(R^{50a})₂, and —N(R^{50a})S(O)₂R^{50b};
R^{50a} in each occurrence may be independently selected from H, C₁₋₆alkyl, carbocyclyl, and heterocyclyl;
R^{50b} in each occurrence may be independently selected from C₁₋₆alkyl, C₂₋₆alkynyl, carbocyclyl, and heterocyclyl.

In yet another aspect, R⁵ may be selected from H and C₁₋₆alkyl, wherein said C₁₋₆alkyl may be optionally substituted with one or more R⁵⁰;

R⁵⁰ in each occurrence may be independently selected from halo, —CN, —OR^{50a}, —SR^{50a}, —N(R^{50a})₂, and —C(O)N(R^{50a})₂; and
R^{50a} in each occurrence may be independently selected from H and C₁₋₆alkyl.

In a further aspect, R⁵ may be C₁₋₆alkyl, wherein said C₁₋₆alkyl is optionally substituted with one or more R⁵⁰;

R⁵⁰ in each occurrence may be independently selected from halo, —CN, —OR^{50a}, —SR^{50a}, —N(R^{50a})₂; and
R^{50a} in each occurrence may be independently selected from H and C₁₋₆alkyl.

In still a further aspect, R⁵ may be selected from H and C₁₋₆alkyl, wherein said C₁₋₆alkyl may be optionally substituted with one or more —OR⁵⁰; and

### R⁵⁰ may be H.

In yet a further aspect, R⁵ may be selected from H, methyl, and hydroxymethyl.

In one aspect, R⁵ may be H.

In another aspect, R⁵ may be methyl.

In still another aspect, R⁵ may be hydroxymethyl.

### Ring A, R¹, R², R³, R⁴, and R⁵

In one aspect, Ring A may be selected from 6-membered heterocyclyl, wherein said 6-membered heterocyclyl may be optionally substituted with one or more R⁶;

R¹ may be selected from —CN, C₁₋₆alkyl, C₂₋₆alkenyl, C₂₋₆alkynyl, 3- to 5-membered carbocyclyl, 5-membered heterocyclyl, —OR^{1a}, —N(R^{1a})₂, —N(R^{1a})C(O)R^{1b}, —NO₂, —C(O)H, —C(O)R^{1b}, —C(O)₂R^{1a}, —C(O)N(R^{1a})₂, —OC(O)R^{1b}, —N(R^{1a})C(O)N(R^{1a})₂, —S(O)R^{1b}, —S(O)₂R^{1b}, —S(O)₂N(R^{1a})₂, and —N(R^{1a})S(O)₂R^{1b}, wherein said C₁₋₆alkyl, C₂₋₆alkenyl, C₂₋₆alkynyl, 3- to 5-membered carbocyclyl, and 5-membered heterocyclyl may be optionally substituted with one or more R¹⁰;
R^{1a} in each occurrence may be independently selected from H, C₁₋₆alkyl, 3- to 5-membered carbocyclyl, and 5-membered heterocyclyl, wherein said C₁₋₆alkyl, 3- to 5-membered carbocyclyl, and 5-membered heterocyclyl in each occurrence may be optionally and independently substituted with one or more R¹⁰;
R^{1b} in each occurrence may be independently selected from C₁₋₆alkyl, C₂₋₆alkenyl, C₂₋₆alkynyl, 3- to 5-membered carbocyclyl, and 5-membered heterocyclyl wherein said C₁₋₆alkyl, C₂₋₆alkenyl, C₂₋₆alkynyl, 3- to 5-membered carbocyclyl, and 5-membered heterocyclyl in each occurrence may be optionally and independently substituted with one or more R¹⁰;
R² may be selected from H, halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —NO₂, —C(O)H, —C(O)R^{2b}, —C(O)₂R^{2a}, —C(O)N(R^{2a})₂, —OC(O)R^{2a}, —N(R^{2a})C(O)N(R^{2a})₂, —S(O)R^{2b}, —S(O)₂R^{2b}, —S(O)₂N(R^{2a})₂, and —N(R^{2a})S(O)₂R^{2b}, wherein said C₁₋₆alkyl, C₂₋₆alkenyl, and C₂₋₆alkynyl, carbocyclyl, and heterocyclyl may be optionally substituted with one or more R²⁰;
R^{2a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R²⁰;
R^{2b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R²⁰;
R³ may be selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —C(O)R^{3b}, —NO₂, —C(O)H, —C(O)₂R^{3a}, —C(O)N(R^{3a})₂, —OC(O)R^{2a}, —N(R^{3a})C(O)N(R^{3a})₂, —S(O)R³¹, —S(O)₂R^{3b}, —S(O)₂N(R^{3a})₂, and —N(R^{3a})S(O)₂R^{3b}, wherein said C₁₋₆alkyl, C₂₋₆alkenyl, and C₂₋₆alkynyl, carbocyclyl, and heterocyclyl may be optionally substituted with one or more R³⁰;
R^{3a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R³⁰;
R^{3b} in each occurrence may be independently selected from C₁₋₆alkyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R³⁰;
R⁴ may be selected from H, C₁₋₆alkyl, and —OR^{4a};
R^{4a} may be selected from H and C₁₋₆alkyl;
R⁵ may be selected from H, —CN, C₁₋₆alkyl, C₂₋₆alkenyl, and C₂₋₆alkynyl, —N(R^{5a})C(O)R^{5b}, —N(R^{5a})N(R^{5a})₂, —NO₂, —C(O)H, —C(O)R^{5b}, —C(O)₂R^{5a}, —C(O)N(R^{5a})₂, —OC(O)N(R^{5a})₂, —N(R^{5a})C(O)₂R^{5a}, —N(R^{5a})C(O)N(R^{5a})₂, —OC(O)R^{5b}, —S(O)R^{5b}, —S(O)₂R^{5b}, —S(O)₂N(R^{5a})₂, —N(R^{5a})S(O)₂R^{5b}, —C(R^{5a})═N(R^{5a}), and —C(R^{5a})═N(OR^{5a}), wherein said C₁₋₆alkyl, C₂₋₆alkenyl, and C₂₋₆alkynyl may be optionally substituted with one or more R⁵⁰;
R^{5a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R⁵⁰;
R^{5b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R⁵⁰;
R⁶ in each occurrence may be independently selected from halo, —CN, C₁₋₆alkyl, C₂₋₆alkenyl, C₂₋₆alkynyl, carbocyclyl, and heterocyclyl, —OR^{6a}, —SR^{6a}, —N(R^{6a})₂, —N(R^{6a})C(O)R^{6b}, —NO₂, —C(O)H, —C(O)R^{6b}, —C(O)₂R^{6a}, —C(O)N(R^{6a})₂, —OC(O)R^{6a}, —N(R^{6a})C(O)N(R^{6a})₂, —S(O)R^{6b}, —S(O)₂R^{6b}, —S(O)₂N(R^{6a})₂, and —N(R^{6a})S(O)₂R^{6b}, wherein said C₁₋₆alkyl, C₂₋₆alkenyl, C₂₋₆alkynyl, carbocyclyl, and heterocyclyl may be optionally substituted with one or more R⁶⁰;
R^{6a} in each occurrence may be independently selected from H, C₁₋₆alkyl, carbocyclyl, and heterocyclyl, wherein said C₁₋₆alkyl, carbocyclyl, and heterocyclyl in each occurrence may be optionally and independently substituted with one or more R⁶⁰;
R^{6b} in each occurrence may be independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence may be optionally and independently substituted with one or more R⁶⁰;
R¹⁰ in each occurrence may be independently selected from halo, —CN, C₁₋₆alkyl, C₂₋₆alkenyl, C₂₋₆alkynyl —OR^{10a}, —SR^{10a}, —N(R^{10a})₂, —N(R^{10a})C(O)R^{10b}, —NO₂, —C(O)H, —C(O)R^{1ob}, —C(O)₂R^{10a}, —C(O)N(R^{10a})₂, —OC(O)R^{10b}, —N(R^{10a})C(O)N(R^{10a})₂, —S(O)R^{10b}, —S(O)₂R^{10b}, —S(O)₂N(R^{10a})₂, and —N(R^{10a})S(O)₂R^{10b};
R^{10a} in each occurrence may be independently selected from H and C₁₋₆alkyl;
R^{10b} in each occurrence may be independently selected from C₁₋₆alkyl, C₂₋₆alkenyl, and C₂₋₆alkynyl;
R²⁰ in each occurrence may be independently selected from halo, —CN, —OR^{20a}, —SR^{20a}, —N(R^{20a})₂, —N(R^{20a})C(O)R^{20b}, —NO₂, —C(O)H, —C(O)R^{20b}, —C(O)₂R^{20a}, —C(O)N(R^{20a})₂, —OC(O)R^{20a}, —N(R^{20a})C(O)N(R^{20a})₂, —S(O)R^{20b}, —S(O)₂R^{20b}, —S(O)₂N(R^{20a})₂, and —N(R^{20a})S(O)₂R^{20b};
R^{20a} in each occurrence may be independently selected from H, C₁₋₆alkyl, carbocyclyl, and heterocyclyl;
R^{20b} in each occurrence may be independently selected from C₁₋₆alkyl, C₂₋₆alkenyl, C₂₋₆alkynyl, carbocyclyl, and heterocyclyl;
R³⁰ in each occurrence may be independently selected from halo, —CN, —OR^{30a}, —SR^{30a}, —N(R^{30a})₂, —N(R^{30a})C(O)R^{30b}, —NO₂, —C(O)H, —C(O)R^{30b}, —C(O)₂R^{30a}, —C(O)N(R^{30a}), —OC(O)R^{30a}, —N(R^{30a})C(O)N(R^{30a})₂, —S(O)R^{30b}, —S(O)²R^{30b}, —S(O)₂N(R^{30a})₂, and —N(R^{30a})S(O)₂R^{30b};
R^{30a} in each occurrence may be independently selected from H, C₁₋₆alkyl, carbocyclyl, and heterocyclyl;
R^{30b} in each occurrence may be independently selected from C₁₋₆alkyl, C₂₋₆alkenyl, C₂₋₆alkynyl, carbocyclyl, and heterocyclyl;
R⁵⁰ in each occurrence may be independently selected from halo, —CN, carbocyclyl, heterocyclyl, —OR^{50a}, —SR^{50a}, —N(R^{50a})₂, —N(R^{50a})C(O)R^{50b}, —NO₂, —C(O)H, —C(O)R^{50b}, —C(O)₂R^{50a}, —C(O)N(R^{50a})₂, —OC(O)R^{50a}, —N(R^{50a})C(O)N(R^{50a})₂, —S(O)R^{50b}, —S(O)₂R^{50b}, —S(O)₂N(R^{50a})₂, and —N(R^{50a})S(O)₂R^{50b};
R^{50a} in each occurrence may be independently selected from H, C₁₋₆alkyl, carbocyclyl, and heterocyclyl;
R^{50b} in each occurrence may be independently selected from C₁₋₆alkyl, C₂₋₆alkenyl, C₂₋₆alkynyl, carbocyclyl, and heterocyclyl;
R⁶⁰ in each occurrence may be independently selected from halo, —CN, C₁₋₆alkyl, C₂₋₆alkenyl, C₂₋₆alkynyl, carbocyclyl, and heterocyclyl, —OR^{60a}, —SR^{60a}, —N(R^{60a})₂, —N(R^{60a})C(O)R^{60b}, —NO₂, —C(O)H, —C(O)R^{60b}, —C(O)₂R^{60a}, —C(O)N(R^{60a})₂, —OC(O)R^{60a}, —N(R^{60a})C(O)N(R^{60a})₂, —S(O)R^{60b}, —S(O)₂R^{60b}, —S(O)₂N(R^{60a})₂, and N(R^{60a})S(O)₂R^{60b};
R^{60a} in each occurrence may be independently selected from H, C₁₋₆alkyl, carbocyclyl, and heterocyclyl; and
R^{60b} in each occurrence may be independently selected from C₁₋₆alkyl, C₂₋₆alkenyl, C₂₋₆alkynyl, carbocyclyl, and heterocyclyl.

In another aspect, Ring A may be selected from 6-membered heterocyclyl, wherein said 6-membered heterocyclyl may be optionally substituted with one or more R⁶;

R¹ may be selected from —CN, C₁₋₆alkyl, C₂₋₆alkenyl, C₂₋₆alkynyl, 3- to 5-membered carbocyclyl, 5-membered heterocyclyl, —OR^{1a}, —N(R^{1a})₂, —N(R^{1a})C(O)R^{1b}, —NO₂, —C(O)H, —C(O)R^{1b}, —C(O)₂R^{1a}, —C(O)N(R^{1a})₂, —OC(O)R^{1b}, —N(R^{1a})C(O)N(R^{1a})₂, —S(O)R^{1b}, —S(O)₂R^{1b}, —S(O)₂N(R^{1a})₂, and —N(R^{1a})S(O)₂R^{1b};
R^{1a} in each occurrence may be independently selected from H, C₁₋₆alkyl, 3- to 5-membered carbocyclyl, and 5-membered heterocyclyl;
R^{1b} in each occurrence may be independently selected from C₁₋₆alkyl, C₂₋₆alkenyl, C₂₋₆alkynyl, 3- to 5-membered carbocyclyl, and 5-membered heterocyclyl;
R² may be selected from H, halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —NO₂, —C(O)H, —C(O)R^{2b}, —C(O)₂R^{2a}, —C(O)N(R^{2a})₂, —OC(O)R^{2a}, —N(R^{2a})C(O)N(R^{2a})₂, —S(O)R^{2b}, —S(O)₂R^{2b}, —S(O)₂N(R^{2a})₂, and —N(R^{2a})S(O)₂R^{2b};
R^{2a} in each occurrence is independently selected from 1-1, C₁₋₆alkyl, carbocyclyl, and heterocyclyl;
R^{2b} is independently selected from C₁₋₆alkyl, C₂₋₆alkenyl, C₂₋₆alkynyl, carbocyclyl, and heterocyclyl;
R³ may be selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —NO₂, —C(O)H, —C(O)R^{3b}, —C(O)₂R^{3a}, —C(O)N(R^{3a})₂, —OC(O)R^{3a}, —N(R^{3a})C(O)N(R^{3a})₂, —S(O)R^{3b}, —S(O)₂R^{3b}, —S(O)₂N(R^{3a})₂, and —N(R^{3a})S(O)₂R^{3b};
R^{3a} in each occurrence is independently selected from 1-1, C₁₋₆alkyl, carbocyclyl, and heterocyclyl;
R^{3b} is independently selected from C₁₋₆alkyl, C₂₋₆alkenyl, C₂₋₆alkynyl, carbocyclyl, and heterocyclyl;
R⁴ may be selected from H, C₁₋₆alkyl, and —OR^{4a};
R^{4a} may be selected from H and C₁₋₆alkyl;
R⁵ may be selected from H, C₁₋₆alkyl, C₂₋₆alkenyl, C₂₋₆alkynyl, wherein said C₁₋₆alkyl, C₂₋₆alkenyl, and C₂₋₆alkynyl may be optionally substituted with one or more R⁵⁰;
R⁶ in each occurrence may be independently selected from halo, —CN, C₁₋₆alkyl, C₂₋₆alkenyl, C₂₋₆alkynyl, carbocyclyl, and heterocyclyl, —OR^{6a}, —SR^{6a}, —N(R^{6a})₂, —N(R^{6a})C(O)R^{6b}, —NO₂, —C(O)H, —C(O)R^{6b}, —C(O)₂R^{6a}, —C(O)N(R^{6a})₂, —OC(O)R^{6a}, —N(R^{6a})C(O)N(R^{6a})₂, —S(O)R^{6b}, —S(O)₂R^{6b}, —S(O)₂N(R^{6a})₂, and —N(R^{6a})S(O)₂R^{6b};
R^{6a} in each occurrence may be independently selected from H, C₁₋₆alkyl, carbocyclyl, and heterocyclyl;
R^{6b} in each occurrence may be independently selected from C₁₋₆alkyl, C₂₋₆alkenyl, C₂₋₆alkynyl, carbocyclyl, and heterocyclyl;
R⁵⁰ in each occurrence may be independently selected from halo, —CN, carbocyclyl, heterocyclyl, —OR^{50a}, —SR^{50a}, —N(R^{50a})₂, —N(R^{50a})C(O)R^{50b}, —NO₂, —C(O)H, —C(O)R^{50b}, —C(O)₂R^{50a}, —C(O)N(R^{50a})₂, —OC(O)R^{50a}, —N(R^{50a})C(O)N(R^{50a})₂, —S(O)R^{50b}, —S(O)₂R^{50b}, —S(O)₂N(R^{50a})₂, and —N(R^{50a})S(O)₂R^{50b};
R^{50a} in each occurrence may be independently selected from H, C₁₋₆alkyl, carbocyclyl, and heterocyclyl; and
R^{50b} in each occurrence may be independently selected from C₁₋₆alkyl, C₂₋₆alkenyl, C₂₋₆alkynyl, carbocyclyl, and heterocyclyl.

In still another aspect, Ring A may be selected from 6-membered heterocyclyl, wherein said 6-membered heterocyclyl may be optionally substituted with one or more R⁶;

R¹ may be selected from —CN, C₁₋₆alkyl, —N(R^{1a})₂, 3- to 5-membered carbocyclyl, and 5-membered heterocyclyl;
R^{1a} in each occurrence may be independently selected from H and C₁₋₆alkyl;
R² may be selected from H, halo, and C₁₋₆alkyl;
R³ may be selected from halo, —CN, —OR^{3a}, —SR^{3a}, and —N(R^{3a});
R^{6a} in each occurrence may be independently selected from H and C₁₋₆alkyl;
R⁴ may be selected from H, C₁₋₆alkyl, and —OR^{4a};
R^{4a} may be selected from H and C₁₋₆alkyl;
R⁵ may be selected from H and C₁₋₆alkyl, wherein said C₁₋₆alkyl may be optionally substituted with one or more R⁵⁰;
R⁶ in each occurrence may be independently selected from halo, —CN, —OR^{6a}, —SR^{6a}, and —N(R^{6a}),
R^{6a} in each occurrence may be independently selected from H and C₁₋₆alkyl;
R⁵⁰ in each occurrence may be independently selected from halo, —CN, —OR^{50a}, —SR^{50a}, —N(R^{50a})₂, and —C(O)N(R^{50a})₂; and
R^{50a} in each occurrence may be independently selected from H and C₁₋₆alkyl.

In yet another aspect, Ring A may be selected from 6-membered heteroaryl, wherein said 6-membered heteroaryl may be optionally substituted with one or more R⁶;

R¹ may be selected from C₁₋₆alkyl, —OR^{1a}, and 3- to 5-membered carbocyclyl;
R^{1a} may be C₁₋₆alkyl;
R² may be selected from H and halo.

### R³ may be H;

R⁴ may be selected from H, C₁₋₆alkyl, and hydroxy;
R⁵ may be selected from H and C₁₋₆alkyl, wherein said C₁₋₆alkyl may be optionally substituted with one or more —OR⁵⁰;
R⁶ may be halo; and

### R⁵⁰ may be H.

In a further aspect, Ring A may be selected from 5-fluoropyridin-2-yl, 3,5-difluoropyridin-2-yl, and 5-fluoropyrimidin-2-yl;

R¹ may be selected from methyl, cyclopropyl, methoxy, ethoxy, and isopropoxy;
R² may be selected from H and fluoro;

### R³ may be H;

R⁴ may be selected from H, methyl, and hydroxy; and
R⁵ may be selected from H, methyl, and hydroxymethyl.

In still a further aspect, compounds of Formula (I) may be compounds of Formula (Ia):

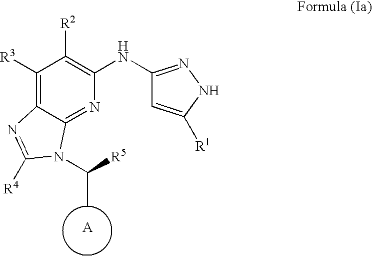

or a pharmaceutically acceptable salt thereof, wherein Ring A, R¹, R², R³, R⁴, and R⁵ are as defined hereinabove.

In one aspect of the invention, the present invention provides a compound of Formula (I), or a pharmaceutically acceptable salt thereof, as illustrated by the Examples, each of which provides a further independent aspect of the invention.

### Utility

### JAK2

The compounds of Formula (I) have utility for the treatment of myeloproliferative disorders, myelodysplastic syndrome and cancer by inhibiting the tyrosine kinases, particularly the JAK family and more particularly JAK2. Methods of treatment target tyrosine kinase activity, particularly the JAK family activity and more particularly JAK2 activity, which is involved in a variety of myeloproliferative disorders, myelodysplastic syndrome and cancer related processes. Thus, inhibitors of tyrosine kinase, particularly the JAK family and more particularly JAK2, are expected to be active against myeloproliferative disorders such as chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and neoplastic disease such as carcinoma of the breast, ovary, lung, colon, prostate or other tissues, as well as leukemias, myelomas and lymphomas, tumors of the central and peripheral nervous system, and other tumor types such as melanoma, fibrosarcoma and osteosarcoma. Tyrosine kinase inhibitors, particularly the JAK family inhibitors and more particularly JAK2 inhibitors are also expected to be useful for the treatment other proliferative diseases including but not limited to autoimmune, inflammatory, neurological, and cardiovascular diseases.

The compounds of Formula (I) have been shown to inhibit tyrosine kinases, particularly the JAK family and more particularly JAK2, as determined by the JAK2 Assay described herein.

The compounds of Formula (I) should also be useful as standards and reagents in determining the ability of a potential pharmaceutical to inhibit tyrosine kinases, particularly the JAK family and more particularly JAK2. These would be provided in commercial kits comprising a compound of this invention.

JAK2 kinase activity was determined by measuring the kinase's ability to phosphorylate synthetic tyrosine residues within a generic polypeptide substrate using an Amplified Luminescent Proximity Assay (Alphascreen) technology (PerkinElmer, 549 Albany Street, Boston, Mass.).

To measure JAK2 kinase activity, a commercially available purified enzyme may be used. The enzyme may be a C-terminal His6-tagged, recombinant, human JAK2, amino acids 808-end, (Genbank Accession number NM 004972) expressed by baculovirus in Sf21 cells (Upstate Biotechnology MA). After incubation of the kinase with a biotinylated substrate and adenosine triphosphate (ATP) for 60 minutes at room temperature, the kinase reaction may be stopped by the addition of 30 mM ethylenediaminetetraacetic acid (EDTA). The reaction may be performed in 384 well microtitre plates and the reaction products may be detected with the addition of streptavidin coated Donor Beads and phosphotyrosine-specific antibodies coated Acceptor Beads using the EnVision Multilabel Plate Reader after an overnight incubation at room temperature.

| | |
|---|---|
| Peptide substrate | TYK2 (Tyr 1054/1055 biotinylated peptide) Cell |
| | Signalling Technology #2200B. 402 μM stock. |
| ATP Km | 30 μM |
| Assay conditions | 150pM JAK2 enzyme, 30 μM ATP, 80 nM Tyk2, |
| | 10 mM MgCl₂, 50 mM Hepes buffer pH 7.5, |
| | 1 mM DTT, 0.025% DTT. |
| Incubation | 60 minutes, room temperature |
| Termination/Detection | 6.3 mM HEPES, 30 mM EDTA, 525 μg/ml |
| conditions | BSA, 40 mM NaCl, 0.007% Triton ® |
| | X-100, 12 ng/ml of Donor Beads, |
| | 12 ng/ml of Acceptor Beads |
| Detection incubation | overnight, room temperature |
| Fluometer settings | Excitation = 680 nm Emission = 570 nm |
| | Excitation Time = 180 ms Total Measurement |
| | Time = 550 ms |

Although the pharmacological properties of the compounds of Formula (I) vary with structural change, in general activity possessed by compounds of Formula (I) may be demonstrated at IC₅₀ concentrations (concentrations to achieve 50% inhibition) or doses at a level below 10 μM.

When tested in the above in-vitro assay the JAK inhibitory activity of the following example was measured at the following IC₅₀.

| Ex | IC₅₀ (μM) |
|---|---|
| 3 | 0.011 |

### TRK

The compounds of Formula (I) have utility for the treatment of cancer by inhibiting the tyrosine kinases, particularly the Trks and more particularly Trk A and B. Methods of treatment target tyrosine kinase activity, particularly the Trk activity and more particularly Trk A and B activity, which is involved in a variety of cancer related processes. Thus, inhibitors of tyrosine kinase, particularly the Trks and more particularly Trk A and B, are expected to be active against neoplastic disease such as carcinoma of the breast, ovary, lung, colon, prostate or other tissues, as well as leukemias and lymphomas, tumors of the central and peripheral nervous system, and other tumor types such as melanoma, fibrosarcoma and osteosarcoma. Tyrosine kinase inhibitors, particularly the Trk inhibitors and more particularly Trk A and B inhibitors are also expected to be useful for the treatment other proliferative diseases including but not limited to autoimmune, inflammatory, neurological, and cardiovascular diseases.

In addition, the compounds of the invention are expected to be of value in the treatment or prophylaxis of cancers selected with up regulated of constitutively activated Trk kinases, including but not limited to, oncogenic rearrangements leading to ETV6-TrkC fusions, TRP-TrkA fusions proteins, AML-ETO (t8;21), autocrine or paracrine signalling leading to elevated serum levels of NGF, BDNF, neurotropins or tumors with constitutively active Trk associated with disease aggressiveness, tumor growth and proliferation or survival signalling.

Compounds of the present invention have been shown to inhibit tyrosine kinases, particularly the Trks and more particularly Trk A and B, as determined by the Trk A Assay described herein.

Compounds provided by this invention should also be useful as standards and reagents in determining the ability of a potential pharmaceutical to inhibit tyrosine kinases, particularly the Trks and more particularly Trk A and B. These would be provided in commercial kits comprising a compound of this invention.

Trk A kinase activity was determined by measuring the kinase's ability to phosphorylate synthetic tyrosine residues within a generic polypeptide substrate using an Amplified Luminescent Proximity Assay (Alphascreen) technology (PerkinElmer, 549 Albany Street, Boston, Mass.).

To measure Trk A kinase activity, the intracellular domain of a HIS-tagged human Trk A kinase (amino acids 442-796 of Trk A, Swiss-Prot Primary Accession Number P04629) may be expressed in SF9 cells and purified using standard nickel column chromatography. After incubation of the kinase with a biotinylated substrate and adenosine triphosphate (ATP) for 20 minutes at room temperature, the kinase reaction may be stopped by the addition of 30 mM ethylenediaminetetraacetic acid (EDTA). The reaction may be performed in 384 well microtitre plates and the reaction products may be detected with the addition of strepavidin coated Donor Beads and phosphotyrosine-specific antibodies coated Acceptor Beads using the EnVision Multilabel Plate Reader after an overnight incubation at room temperature.

| | |
|---|---|
| Peptide substrate | PolyEY-biotin (PGT-bio.) |
| ATP Km | 70 μM |
| Assay conditions | 0.838 ng/ml Trk A, 9 mM HEPES, |
| | 45 μg/ml BSA, 10 mM MnCl₂, 5 nM PGT-bio, |
| | 0.01% Triton ® X-100, 70 μM ATP |
| Incubation | 20 minutes, room temperature |
| Termination/Detection | 6.3 mM HEPES, 30 mM EDTA, |
| conditions | 525 μg/ml BSA, 40 mM NaCl, 0.007% |
| | Triton ® X-100, 12 ng/ml of Donor Beads, |
| | 12 ng/ml of Acceptor Beads |
| Detection incubation | overnight, room temperature |
| Fluometer settings | Excitation = 680 nm Emission = 570 nm |
| | Excitation Time = 180 ms Total Measurement |
| | Time = 550 ms |

Although the pharmacological properties of the compounds of Formula (I) vary with structural change, in general activity possessed by compounds of Formula (I) may be demonstrated at ICₛₒ concentrations (concentrations to achieve 50% inhibition) or doses at a level below 10 μM.

When tested in the above in-vitro assay the Trk inhibitory activity of the following example was measured at the following IC₅₀s.

| Ex | IC₅₀ (μM) |
|---|---|
| 1 | 0.003 |
| 2 | 0.023 |
| 3 | 0.003 |
| 4 | 0.003 |
| 5 | 0.003 |
| 6 | 0.003 |
| 7 | 0.003 |
| 8 | 0.003 |
| 9 | 0.003 |
| 10 | 0.003 |
| 11 | 0.003 |
| 12 | 0.066 |
| 13 | 0.021 |
| 14 | 3.296 |
| 15 | 9.347 |
| 16 | 0.073 |
| 17 | 0.283 |
| 18 | 0.393 |

Thus, in one aspect, there is provided a compound of Formula (I), or a pharmaceutically acceptable salt thereof, for use as a medicament.

In another aspect, there is provided the use of a compound of Formula (I), or a pharmaceutically acceptable salt thereof, in the manufacture of a medicament for the treatment or prophylaxis of myeloproliferative disorders, myelodysplastic syndrome, and cancer, in a warm-blooded animal such as man.

In still another aspect, there is provided the use of a compound of Formula (I), or a pharmaceutically acceptable salt thereof, in the manufacture of a medicament for the treatment or prophylaxis of myeloproliferative disorders, myelodysplastic syndrome and cancers (solid and hematologic tumors), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acromegaly, acute and chronic inflammation, bone diseases, and ocular diseases with retinal vessel proliferation, in a warm-blooded animal such as man.

In yet another aspect, there is provided the use of a compound of Formula (I), or a pharmaceutically acceptable salt thereof, in the manufacture of a medicament for treating chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia, in a warm-blooded animal such as man.

In a further aspect, there is provided the use of a compound of Formula (I), or a pharmaceutically acceptable salt thereof, in the manufacture of a medicament for the production of an anti-proliferative effect, in a warm-blooded animal such as man.

In still a further aspect, there is provided the use of a compound of Formula (I), or a pharmaceutically acceptable salt thereof, in the manufacture of a medicament for the production of a JAK inhibitory effect.

In yet a further a further aspect, there is provided the use of a compound of Formula (I), or a pharmaceutically acceptable salt thereof, in the manufacture of a medicament for the production of a TRK inhibitory effect.

In one aspect, there is provided the use of a compound of Formula (I), or a pharmaceutically acceptable salt thereof, in the manufacture of a medicament for the treatment of cancer.

In another aspect, there is provided a method of treating myeloproliferative disorders, myelodysplastic syndrome, and cancer, in a warm-blooded animal such as man, said method comprising administering to said animal an effective amount of a compound of Formula (I), or a pharmaceutically acceptable salt thereof.

In still another aspect, there is provided a method of treating myeloproliferative disorders, myelodysplastic syndrome, and cancers (solid and hematologic tumors), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acromegaly, acute and chronic inflammation, bone diseases, and ocular diseases with retinal vessel proliferation, in a warm-blooded animal such as man, said method comprising administering to said animal an effective amount of a compound of Formula (I), or a pharmaceutically acceptable salt thereof.

In yet another aspect, there is provided a method of treating chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia, in a warm-blooded animal such as man, said method comprising administering to said animal an effective amount of compound of Formula (I), or a pharmaceutically acceptable salt thereof.

In a further aspect, there is provided a method for producing an anti-proliferative effect in a warm-blooded animal such as man, said method comprising administering to said animal an effective amount of a compound of Formula (I), or a pharmaceutically acceptable salt thereof.

In still a further aspect, there is provided a method for producing a JAK inhibitory effect in a warm-blooded animal such as man, said method comprising administering to said animal an effective amount of a compound of Formula (I), or a pharmaceutically acceptable salt thereof.

In yet a further aspect, there is provided a method for producing a TRK inhibitory effect in a warm-blooded animal such as man, said method comprising administering to said animal an effective amount of a compound of Formula (I), or a pharmaceutically acceptable salt thereof.

In one aspect, there is provided a method for treating cancer in a warm-blooded animal such as man, said method comprising administering to said animal an effective amount of a compound of Formula (I), or a pharmaceutically acceptable salt thereof.

In another aspect, there is provided a compound of Formula (I), or a pharmaceutically acceptable salt thereof, for use in treating myeloproliferative disorders, myelodysplastic syndrome, and cancer, in a warm-blooded animal such as man.

In still another aspect, there is provided a compound of Formula (I), or a pharmaceutically acceptable salt thereof, for use in treating myeloproliferative disorders, myelodysplastic syndrome, and cancers (solid and hematologic tumors), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acromegaly, acute and chronic inflammation, bone diseases, and ocular diseases with retinal vessel proliferation, in a warm-blooded animal such as man.

In yet another aspect, there is provided a compound of Formula (I), or a pharmaceutically acceptable salt thereof, for use in the treating chronic myeloid leukemia, polycythemia vera, essential thrombocythemia, myeloid metaplasia with myelofibrosis, idiopathic myelofibrosis, chronic myelomonocytic leukemia and hypereosinophilic syndrome, myelodysplastic syndromes and cancers selected from oesophageal cancer, myeloma, hepatocellular, pancreatic, cervical cancer, Ewings sarcoma, neuroblastoma, Kaposi's sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, mesothelioma, renal cancer, lymphoma and leukaemia, in a warm-blooded animal such as man.

In a further aspect, there is provided a compound of Formula (I), or a pharmaceutically acceptable salt thereof, for use in the production of an anti-proliferative effect, in a warm-blooded animal such as man.

In still a further aspect, there is provided a compound of Formula (I), or a pharmaceutically acceptable salt thereof, for use in the production of a JAK inhibitory effect in a warm-blooded animal such as man.

In yet a further aspect, there is provided a compound of Formula (I), or a pharmaceutically acceptable salt thereof, for use in the production of a TRK inhibitory effect in a warm-blooded animal such as man.

In one aspect, there is provided a compound of Formula (I), or a pharmaceutically acceptable salt thereof, for use in the treatment of cancer in a warm-blooded animal such as man.

In one aspect, where reference is made to the Trk inhibitory effect, this may particularly refer to a Trk A inhibitory effect.

In another aspect, where reference is made to the Trk inhibitory effect, this may particularly refer to a Trk B inhibitory effect.

In still another aspect, where reference is made to the treatment (or prophylaxis) of cancer, it may particularly refer to the treatment (or prophylaxis) of mesoblastic nephroma, mesothelioma, acute myeloblastic leukemia, acute lymphocytic leukemia, multiple myeloma, oesophageal cancer, myeloma, hepatocellular, pancreatic, cervical cancer, Ewings sarcoma, neuroblastoma, Kaposi's sarcoma, ovarian cancer, breast cancer including secretory breast cancer, colorectal cancer, prostate cancer including hormone refractory prostate cancer, bladder cancer, melanoma, lung cancer—non small cell lung cancer (NSCLC), and small cell lung cancer (SCLC), gastric cancer, head and neck cancer, renal cancer, lymphoma, thyroid cancer including papillary thyroid cancer, mesothelioma, leukaemia, tumors of the central and peripheral nervous system, melanoma, fibrosarcoma including congenital fibrosarcoma and osteosarcoma. More particularly it refers to prostate cancer. In addition, more particularly it refers to SCLC, NSCLC, colorectal cancer, ovarian cancer and/or breast cancer. In a further aspect it may refer to hormone refractory prostate cancer.

In still another aspect, there is provided a pharmaceutical composition comprising a compound of Formula (I), or a pharmaceutically acceptable salt thereof, and at least one pharmaceutically acceptable carrier, diluent, or excipient.

In yet another aspect, there is provided a pharmaceutical composition comprising a compound of Formula (I), or a pharmaceutically acceptable salt thereof, and at least one pharmaceutically acceptable carrier, diluent, or excipient.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate; granulating and disintegrating agents such as corn starch or algenic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form or in the form of nano or micronized particles together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example, polyoxethylene stearate), or condensation products of ethylene oxide with long chain aliphatic alcohols (for example, heptadecaethyleneoxycetanol), or condensation products of ethylene oxide with partial esters derived from fatty acids and a hexitol such as polyoxyethylene sorbitol monooleate, or condensation products of ethylene oxide with long chain aliphatic alcohols (for example, heptadecaethyleneoxycetanol), or condensation products of ethylene oxide with partial esters derived from fatty acids and a hexitol such as polyoxyethylene sorbitol monooleate, or condensation products of ethylene oxide with partial esters derived from fatty acids and hexitol anhydrides (for example, polyethylene sorbitan monooleate). The aqueous suspensions may also contain one or more preservatives such as ethyl or propyl p-hydroxybenzoate; anti-oxidants such as ascorbic acid); coloring agents; flavoring agents; and/or sweetening agents such as sucrose, saccharine or aspartame.

Oily suspensions may be formulated by suspending the active ingredient in a vegetable oil such as arachis oil, olive oil, sesame oil or coconut oil or in a mineral oil such as liquid paraffin. The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as, for example liquid paraffin, or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example, sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent (for example, a solution in 1,3-butanediol).

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 4 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. Accordingly, the optimum dosage may be determined by the practitioner who is treating any particular patient.

The anti-cancer treatment defined herein may be applied as a sole therapy or may involve, in addition to the compound of the invention, conventional surgery or radiotherapy or chemotherapy. Such chemotherapy may include one or more of the following categories of anti-tumor agents:
(i) antiproliferative/antineoplastic drugs and combinations thereof, as used in medical oncology, such as alkylating agents (for example, cis-platin, carboplatin, cyclophosphamide, nitrogen mustard, melphalan, chlorambucil, busulphan and nitrosoureas); antimetabolites (for example, antifolates such as fluoropyrimidines like 5-fluorouracil and tegafur, raltitrexed, methotrexate, cytosine arabinoside and hydroxyurea); antitumor antibiotics (for example, anthracyclines such as adriamycin, bleomycin, doxorubicin, daunomycin, epirubicin, idarubicin, mitomycin-C, dactinomycin and mithramycin); antimitotic agents (for example, vinca alkaloids such as vincristine, vinblastine, vindesine and vinorelbine and taxoids such as taxol and taxotere); and topoisomerase inhibitors (for example, epipodophyllotoxins such as etoposide and teniposide, amsacrine, topotecan and camptothecin); and proteosome inhibitors (for example, bortezomib [Velcade®]); and the agent anegrilide [Agrylin®]; and the agent alpha-interferon;
(ii) cytostatic agents such as antioestrogens (for example, tamoxifen, toremifene, raloxifene, droloxifene and iodoxyfene), oestrogen receptor down regulators (for example, fulvestrant), antiandrogens (for example, bicalutamide, flutamide, nilutamide and cyproterone acetate), LHRH antagonists or LHRH agonists (for example, goserelin, leuprorelin and buserelin), progestogens (for example, megestrol acetate), aromatase inhibitors (for example, as anastrozole, letrozole, vorazole and exemestane) and inhibitors of 5α-reductase such as finasteride;
(iii) agents which inhibit cancer cell invasion (for example, metalloproteinase inhibitors such as marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD 1839), N-(3-ethynylphenyl)-6,7-bis (2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family, for example inhibitors or phosphotidylinositol 3-kinase (PI3K) and for example inhibitors of mitogen activated protein kinase (MEK1/2) and for example inhibitors of protein kinase B (PKB/Akt), for example inhibitors of Src tyrosine kinase family and/or Abelson (Abl) tyrosine kinase family such as AZD0530 and dasatinib (BMS-354825) and imatinib mesylate (Gleevec™); and any agents that modify STAT signalling;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies and approaches using the immunomodulatory drugs thalidomide and lenalidomide [Revlimid®]; and
(x) other treatment regimes including: dexamethasone, proteasome inhibitors (including bortezomib), isotretinoin (13-cis retinoic acid), thalidomide, revemid, Rituxamab, ALIMTA, Cephalon's kinase inhibitors CEP-701 and CEP-2563, anti-Trk or anti-NGF monoclonal antibodies, targeted radiation therapy with 131I-metaiodobenzylguanidine (131I-MBG), anti-G(D2) monoclonal antibody therapy with or without granulocyte-macrophage colony-stimulating factor (GM-CSF) following chemotherapy.

Such conjoint treatment may be achieved by way of the simultaneous, sequential or separate dosing of the individual components of the treatment. Such combination products employ the compounds of this invention, or pharmaceutically acceptable salts thereof, within the dosage range described hereinbefore and the other pharmaceutically-active agent within its approved dosage range.

In addition to its use in therapeutic medicine, compounds of Formula (I) and pharmaceutically acceptable salts are also useful as pharmacological tools in the development and standardization of in vitro and in vivo test systems for the evaluation of the effects of inhibitors of JAK2 in laboratory animals such as cats, dogs, rabbits, monkeys, rats and mice, as part of the search for new therapeutic agents.

In any of the above-mentioned pharmaceutical composition, process, method, use, medicament, and manufacturing features of the instant invention, any of the alternate embodiments of the compounds of the invention described herein also apply.

In one aspect, the inhibition of JAK activity particularly refers to the inhibition of JAK2 activity.

### Process

If not commercially available, the necessary starting materials for the procedures such as those described herein may be made by procedures which are selected from standard organic chemical techniques, techniques which are analogous to the synthesis of known, structurally similar compounds, or techniques which are analogous to the Examples, Procedures, and Schemes, described herein.

It is noted that many of the starting materials for synthetic methods as described herein are commercially available and/or widely reported in the scientific literature, or could be made from commercially available compounds using adaptations of processes reported in the scientific literature. The reader is further referred to Advanced Organic Chemistry, 5^{th} Edition, by Jerry March and Michael Smith, published by John Wiley & Sons 2001, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, published by John Wiley and Sons, 1991).

Compounds of Formula (I) may be prepared in a variety of ways. The Processes and Schemes shown below illustrate some methods for synthesizing compounds of Formula (I) and intermediates which may be used for the synthesis of compounds of Formula (I) (wherein Ring A, R¹, R², R³, R⁴, and R⁵, unless otherwise defined, are as defined hereinabove). Where a particular solvent or reagent is shown in a Scheme or referred to in the accompanying text, it is to be understood that the chemist of ordinary skill in the art will be able to modify that solvent or reagent as necessary. The Processes and Schemes are not intended to present an exhaustive list of methods for preparing the compounds of Formula (I); rather, additional techniques of which the skilled chemist is aware may be also be used for the compounds' synthesis. The claims are not intended to be limited to the structures shown in the Processes and Schemes.

The skilled chemist will be able to use and adapt the information contained and referenced within the above references, and accompanying Examples therein and also the Examples, Procedures, and Schemes herein, to obtain necessary starting materials and products.

In one aspect, compounds of Formula (I) may be prepared by:

1) Process A—reacting a compound of Formula (A):

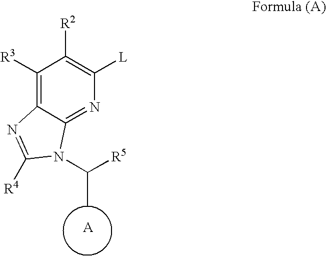

with a compound of Formula (B):

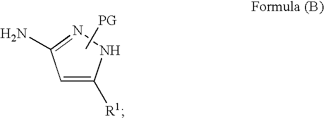

2) Process B—reacting a compound of Formula (C):

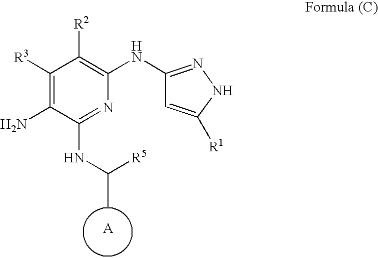

with a compound of Formula (D):

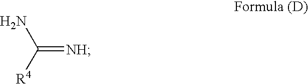

3) Process C—reacting a compound of Formula (E):

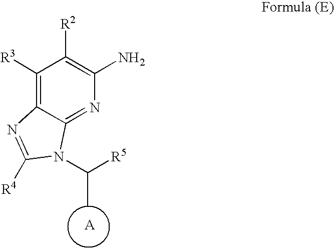

with a compound of Formula (F):

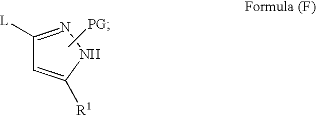

and
4) Process D—reacting a compound of Formula (G):

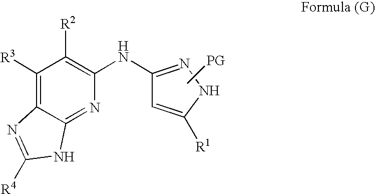

with a compound of Formula (H):

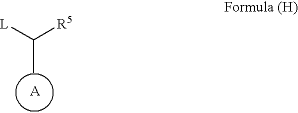

and thereafter if appropriate:
i) converting a compound of Formula (I) into another compound of Formula (I);
ii) removing any protecting groups; and/or
iii) forming a pharmaceutically acceptable salt,
   wherein
   L in each occurrence may be the same or different, and is a leaving group, as discussed hereinabove; and
   PG in each occurrence may be the same or different, and is a protecting group, as discussed hereinabove.

Specific reaction conditions for the Processes shown above are as follows:

Process A—Compounds of Formula (A) and compounds of Formula (B) may be reacted together in the presence of a suitable solvent, examples of which include ketones such as acetone, alcohols such as ethanol and butanol, and aromatic hydrocarbons such as toluene and N-methylpyrrolid-2-one. Such reaction may advantageously occur in the presence of a suitable base examples of which include inorganic bases such as cesium carbonate and potassium carbonate, and organic bases such as triethylamine and diisopropylethylamine. The reaction is advantageously performed at a temperature in a range from 0° C. to reflux.

In another aspect, compounds of Formula (A) and compounds of Formula (B) may be reacted together under standard Buchwald conditions (for example see J. Am. Chem. Soc., 118, 7215; J. Am. Chem. Soc., 119, 8451; J. Org. Chem., 62, 1568 and 6066), with a suitable base. Examples of suitable bases include inorganic bases such as cesium carbonate, and organic bases such as potassium t-butoxide. Such a reaction may be advantageously occur in the presence of palladium acetate. Solvents suitable for such a reaction include aromatic solvents such as toluene, benzene, or xylene.

Process B—Examples of compounds of Formula (D) include formamidine acetate. Other compounds which advantageously may be used in place of the compounds of Formula (D) include orthoesters such as triethyl orthoformate and triethyl orthoacetate.

Process C may be performed under conditions similar to those described for Process A or according to the Buchwald conditions described for process D.

Process D—Compounds of Formula (G) and Formula (H) may be reacted together under standard nucleophilic addition reaction conditions. For example, such a reaction may be performed in the presence of a suitable base such as potassium carbonate and a suitable solvent such as DMF and at a temperature range from about 25° C. to about 100° C.

Compounds of Formula (G) may be prepared according to Scheme 1:

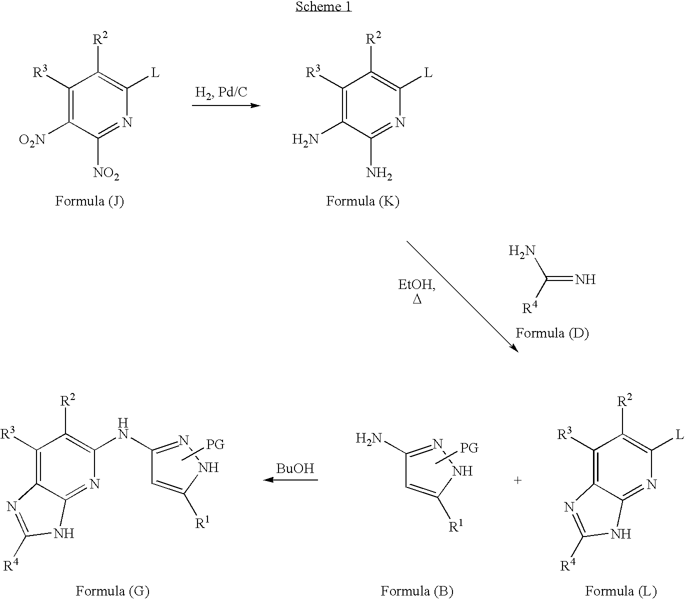

Compounds of Formula (G) may also be prepared according to Scheme 2:

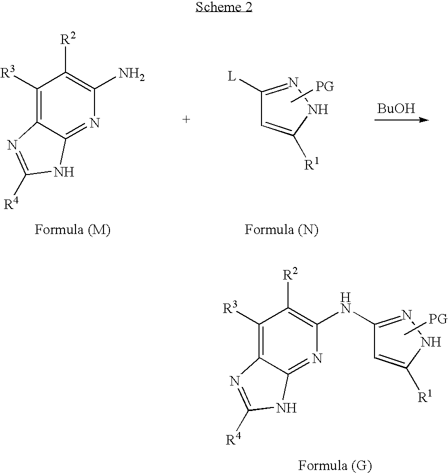

Compounds of Formula (G) may also be prepared according to Scheme 3:

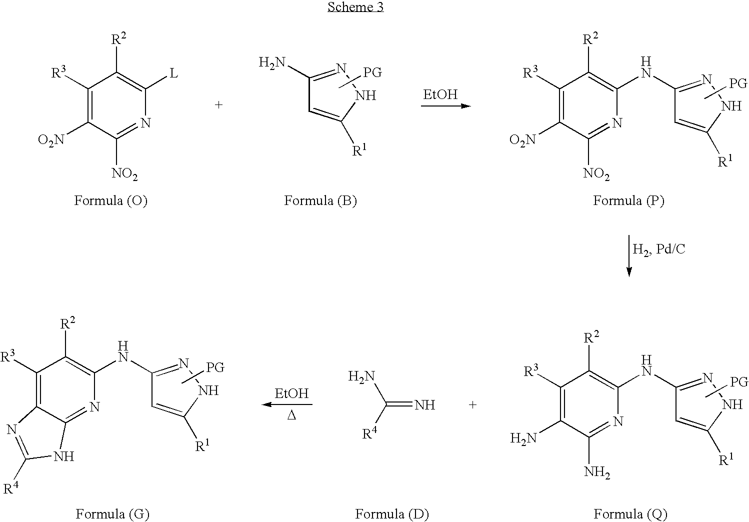

Compounds of Formula (E) may be prepared according to Scheme 4:

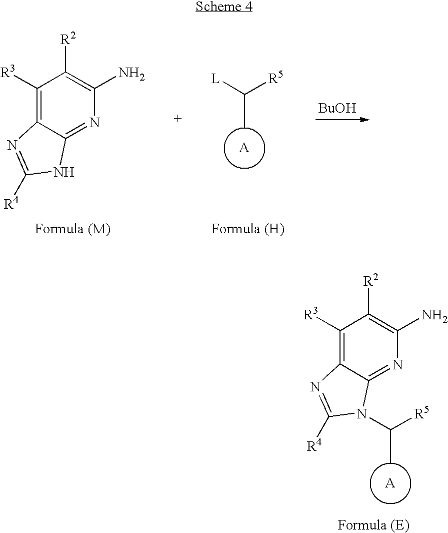

Compounds of Formula (A) may be prepared according to Scheme 5:

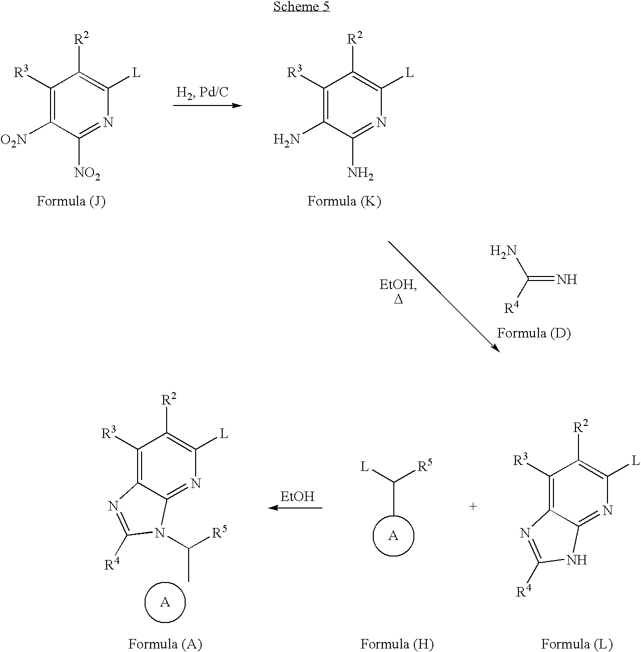

Compounds of Formula (C) may be prepared according to Scheme 6:

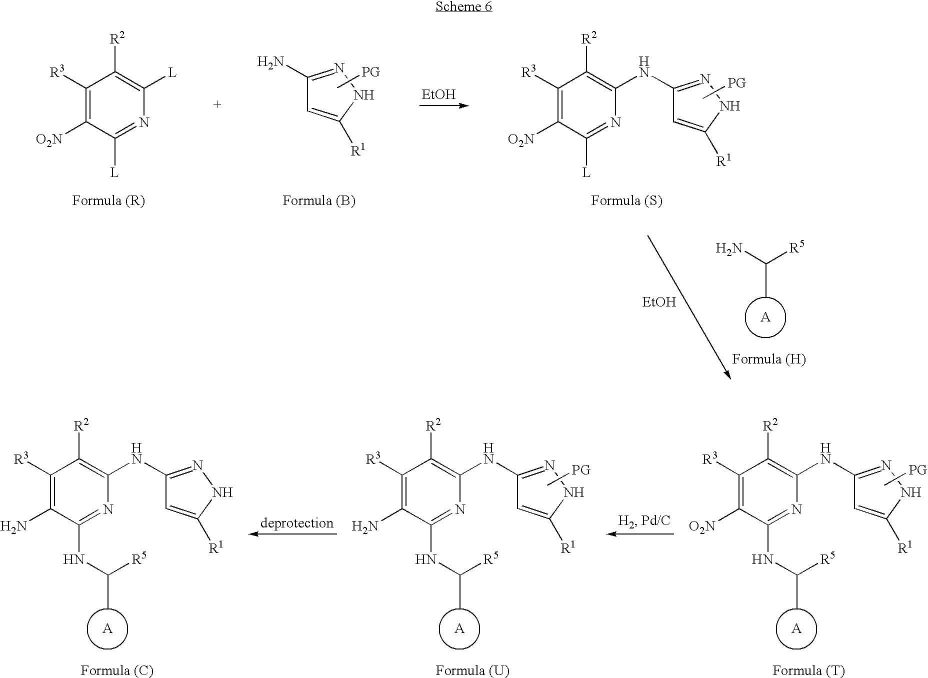

It is to be understood that the reaction conditions shown in Schemes 1 through 6 are meant to be illustrative, and that the skilled chemist will be able to modify the reaction conditions as necessary.

### EXAMPLES

The invention will now be further described with reference to the following illustrative Examples in which, unless stated otherwise:
(i) temperatures are given in degrees Celsius (° C.); operations are carried out at room temperature or ambient temperature, that is, in a range of 18-25° C.;
(ii) organic solutions were dried over anhydrous magnesium sulfate unless other wise stated; evaporation of organic solvent was carried out using a rotary evaporator under reduced pressure (4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC or liquid chromatography/mass spectroscopy and reaction times are given for illustration only;
(v) final products have satisfactory proton nuclear magnetic resonance (NMR) spectra and/or mass spectra data;
(vi) yields are given for illustration only and are not necessarily those which can be obtained by diligent process development; preparations were repeated if more material was required;
(vii) when given, NMR data is in the form of delta values for major diagnostic protons, given in part per million (ppm) relative to tetramethylsilane (TMS) as an internal standard, determined at 300 MHz in DMSO-d₆ unless otherwise stated;
(viii) chemical symbols have their usual meanings;
(ix) solvent ratio was given in volume:volume (v/v) terms.
(x) “ISCO” refers to normal phase flash column chromatography using pre-packed silica gel cartridges (12 g, 40 g etc.), used according to the manufacturer's instructions, obtained from ISCO, Inc, 4700 Superior Street Lincoln, Nebr., USA.
(xi) A “Gilson column” refers to a YMC-AQC18 reverse phase HPLC Column with dimension 20 mm/100 and 50 mm/250 in H₂O/MeCN with 0.1% TFA as mobile phase unless otherwise stated and used according to the manufacturer's instructions, obtained from Gilson, Inc. 3000 Parmenter Street, Middleton, Wis. 53562-0027, U.S.A.
(xii) “Biotage” refers to normal phase flash column chromatography using pre-packed silica gel cartridges (12 g, 40 g, 80 g etc.), used according to the manufacturer's instructions, obtained from Biotage Inc, 1725 Discovery Drive Charlotteville, Va. 22911, USA.
(xiii) “SFC (super critical fluid chromatography)” refers to Analytical SFC (ASC-1000 Analytical SFC System with Diode Array Detector) and/or Preparative SFC (APS-1000 AutoPrep Preparative SFC), used according to the manufacturer's instruction, obtained from SFC Mettler Toledo AutoChem, Inc. 7075 Samuel Morse Drive Columbia Md. 21046, U.S.A.
(xiv) Parr Hydrogenator or Parr shaker type hydrogenators are systems for treating chemicals with hydrogen in the presence of a catalyst at pressures up to 5 atmospheres (60 psi) and temperatures to 80° C.
(xv) the following abbreviations have been used:
   BINAP 2,2′-bis(diphenylphosphino)-1,1′-binapthyl
   Boc₂O di-tert-butyl-dicarbonate
   DCM dichloromethane
   DIPEA N,N-diisopropylethylamine
   DMF N,N-dimethylformamide
   DMAP 4-dimethylaminopyridine
   DMSO dimethylsulfoxide
   dppf 1,1′-Bis(diphenylphosphino)ferrocene
   EtOAc ethyl acetate
   Et₂O diethyl ether
   GC gas chromatography
   HPLC high-performance liquid chromatography
   LCMS liquid chromatography/mass spectroscopy
   Pd₂(dba)₃ tris(dibenzylideneacetone)dipalladium (0)
   THF tetrahydrofuran
   TFA trifluoroacetic acid
   Xantphos 9,9-Dimethyl-4,5-bis(diphenylphosphino)xanthene

### Intermediate 1

### 5-Fluoropyridine-2-carbonitrile

2-Bromo-5-fluoropyridine (93.0 g, 528 mmol), Zn dust (8.29 g, 127 mmol), zinc cyanide (40.3 g, 343 mmol), 1,1′-bis(diphenylphosphino)ferrocene (11.7 g, 21.1 mmol) and Pd₂dba₃ (9.68 g, 10.6 mmol) in anhydrous DMAc (300 ml) was heated at 95° C. for 3 hours. After cooled to room temperature, brine (100 ml) and ether (500 ml) was added. The solid formed was removed by filtration and washed with ether (300 ml). The organic layer was separated, washed with brine (200 ml) and dried over sodium sulfate, and concentrated. After removal of solvent, the resulted residue was purified by column chromatography (hexane:DCM=1:1) to give the title compound as a white solid (49 g, 72%). ¹H NMR (400 MHz) δ 8.82 (d, 1H), 8.21 (dd, 1H), 8.05 (dd, 1H).

### Intermediate 2

### N-(1-(5-Fluoropyridin-2-yl)vinyl)acetamide

A solution of MeMgBr (170.3 ml, 510.98 mmol) in ether was diluted with 170 ml of anhydrous THF and cooled to 0° C. 5-Fluoropyridine-2-carbonitrile (Intermediate 1, 53.6 g, 425.82 mmol) in THF (170 ml) was added drop-wise. The reaction was stirred at 0° C. for 30 minutes, then diluted with dichloromethane (170 ml). Acetic anhydride (48.3 ml, 510.98 mmol) in dichloromethane (100 ml) was added drop-wise at 0° C. After addition, the reaction was warmed to room temperature and stirred at room temperature for 8 hours. Saturated sodium bicarbonate solution (50 ml) was added and extracted with EtOAc (2×200 ml). The combined organic was dried over sodium sulfate. After removal of solvent, the resulted residue was purified by column chromatography (hexane:EtOAc=2.5:1) to give the title compound as a white solid (26.6 g, 35%). ¹H NMR (400 MHz) δ 9.37 (s, 1H), 8.57 (d, J=2.8 Hz, 1H), 7.81 (m, 2H), 6.01 (s, 1H), 5.52 (s, 1H), 2.08 (s, 3H). LCMS: 181 [M+1-1]⁺.

### Intermediate 3

### (S)-N-(1-(5-Fluoropyridin-2-yl)ethyl)acetamide

To a solution of N-(1-(5-fluoropyridin-2-yl)vinyl)acetamide (Intermediate 2, 11.0 g, 61.1 mmol) in MeOH (120 ml) under N₂ was added (+)-1,2-bis((2S,5S)-2,5-diethylphospholano)benzene (cyclooctadiene)rhodium(I)trifluoromethanesulfonate (0.441 g, 0.611 mmol). The solution was transferred to a high-pressure bomb and charged 150 psi H₂. The reaction stirred at room temperature and maintained inside pressure between 120-150 psi for 7 hours. The solvent was removed and the resulted residue was purified by column chromatography (EtOAc) to give the title compound as a white solid (9.8 g, 88%). ¹H NMR (400 MHz) δ 8.49 (d, J=2.4 Hz, 1H), 8.32 (d, 1H), 7.66 (m, 1H), 7.39 (dd, 1H), 4.95 (m, 1H), 1.85 (s, 3H), 1.34 (d, 3H). LCMS: 183 [M+H]⁺. Enantiomeric excess determined by HPLC (Chiralpak IA; 70:30 CO₂/MeOH), 95.3% ee.

### Intermediate 4

### tert-Butyl [(1S)-1-(5-fluoropyridin-2-yl)ethyl]carbamate

A solution of (S)-N-(1-(5-fluoropyridin-2-yl)ethyl)acetamide (Intermediate 3, 11.0 g, 60.37 mmol), DMAc (1.48 g, 12.07 mmol) and Boc₂O (26.35 g, 120.7 mmol) in THF (100 ml) was stirred at 50° C. for 20 hours. After cooled to room temperature, lithium hydroxide monohydrate (5.19 g, 123.8 mmol) and water (100 ml) were added. The reaction was stirred at room temperature for 5 hours and diluted with ether (200 ml). The organic layer was separated, washed with brine (100 ml), and dried over sodium sulfate. After removal of solvent, the resulted residue was purified by column chromatography (Hexane:EtOAc=5:1) to give the title compound as a pale yellow oil (13.6 g, 94%). ¹H NMR (400 MHz) δ 8.46 (d, 1H), 7.69 (m, 1H), 7.35-7.41 (m, 2H), 4.67 (m, 1H), 1.37 (s, 9H), 1.32 (d, 3H). LCMS: 241 [M+H]⁺.

### Intermediate 5

### [(1S)-1-(5-Fluoropyridin-2-yl)ethyl]amine

To a solution of tert-butyl [(1S)-1-(5-fluoropyridin-2-yl)ethyl]carbamate (Intermediate 4, 12.8 g, 53.3 mmol) in DCM (100 ml) was added HCl/dioxane solution (107 ml, 4 N, 428 mmol). The reaction was stirred at room temperature for 3 hours. The solvent was removed and 50 ml of saturated sodium bicarbonate was added. The resulting aqueous solution was extracted with ether (6×400 ml), dried over sodium sulfate and concentrated to give the title compound (7.30 g, 98%) as pale yellow oil. ¹H NMR (400 MHz) δ 8.44 (d, 1H), 7.66 (m, 1H), 7.53 (m, 1H), 4.01 (q, 1H), 1.94 (b, 2H), 1.26 (d, 3H). LCMS: 141 [M+H]⁺.

The hydrochloride salt of [(1S)-1-(5-fluoropyridin-2-yl)ethyl]amine may be prepared by dissolving the title compound in MeOH, and adding to the resulting solution a solution of HCl/dioxane. Evaporation of the solvent provides the hydrochloride salt of the title compound as a tan solid.

### Intermediate 6

### 2,3,6-Trifluoro-5-nitropyridine

To a 3-neck, round-bottomed flask was added 2,3,6-trifluoropyridine (25 g, 0.19 mol) followed by the addition of red fuming nitric acid (210 mL, 4.7 mol). Sulfuric acid (150 mL, 2.8 mol) was added to this mixture slowly via an addition funnel, maintaining internal temperature below 40° C. The resulting solution was heated to 60° C. for 30 minutes and allowed to cool to room temperature after heating. This solution was then further cooled in an ice-water bath and inversely quenched into a 2-L Erlenmeyer flask containing a mixture of ice and water (700 mL, 1:1 ratio). The quenched solution was then transferred to a 2-L separatory funnel and partitioned with hexanes (600 mL). The aqueous layer was subsequently washed with hexanes (600 mL) and methylene chloride (600 mL). The combined organic layers were then dried over Na₂SO₄, filtered, and concentrated to provide the title compound as a light yellow liquid (19.2 g, 57% yield).

¹H NMR (CDCl₃) δ 8.74 (s, 1H).

### Intermediate 7

### 5,6-Difluoro-N-[(1S)-1-(5-fluoropyridin-2-yl)ethyl]-3-nitropyridin-2-amine

To a cold (0° C.) solution of 2,3,6-trifluoro-5-nitropyridine (Intermediate 6, 8.02 g, 45 mmol) and triethylamine (12.5 mL, 90 mmol) in THF (100 mL) was added the hydrochloride salt of [(1S)-1-(5-fluoropyridin-2-yl)ethyl]amine (Intermediate 5, 10 g, 56 mmol) in portions. The reaction was allowed to stir at cold temperature for 1 hour then allowed to warm up to room temperature. The reaction was quenched with saturated NaCl (aq) solution and partitioned with EtOAc. The organic layer was dried over Na₂SO₄, filtered, and concentrated. The residue was purified by flash chromatography (Biotage, 20%→30% EtOAc/hexanes) to provide the title compound as a yellow solid (9.67 g, 72% isolated yield). LCMS (electrospray): 299 [M+1]. ¹H NMR (CDCl₃) δ 8.43 (s, 1H) 7.98-8.09 (m, 1H) 7.38-7.49 (m, 1H) 7.27-7.35 (m, 1H) 7.07 (d, 1H) 5.34 (d, 1H) 1.57 (d, 3H).

### Intermediate 8

### N²-(5-Cyclopropyl-1H-pyrazol-3-yl)-3-fluoro-N⁶-[(1S)-1-(5-fluoropyridin-2-yl)-ethyl]-5-nitropyridine-2,6-diamine

A mixture of 5,6-difluoro-N-[(1S)-1-(5-fluoropyridin-2-yl)ethyl]-3-nitropyridin-2-amine (Intermediate 7, 894 mg, 3 mmol), 5-cyclopropyl-1H-pyrazol-3-amine (740 mg, 6 mmol), and DIPEA (0.7 mL, 3.9 mmol) in THF (20 mL) was heated to 55° C. for 16 hours. The mixture was concentrated and purified by flash chromatography (Biotage, 30%→60% EtOAc/hexanes) to provide the title compound as an orange solid (620 mg). LCMS (electrospray): 402 [M+1]¹H NMR (CDCl₃) δ 11.01 (s, 1H) 8.48 (s, 1H) 8.02 (d, 1H) 7.28-7.51 (m, 2H) 6.66 (s, 1H) 5.38 (t, 1H) 1.86-2.00 (m, 1H) 1.66 (d, 3H) 0.99 (d, 2H) 0.72-0.79 (m, 2H).

### Intermediate 9

### 6-Chloro-N-[(1S)-1-(5-fluoropyridin-2-yl)-ethyl]-3-nitropyridin-2-amine

The title compound was prepared using a procedure analogous to the one described for the synthesis of Intermediate 7, using 2,6-dichloro-3-nitropyridine and the hydrochloride salt of [(1S)-1-(5-fluoropyridin-2-yl)ethyl]amine (Intermediate 5) as the starting materials. The reaction was quenched with saturated NaCl_{(aq)} solution and partitioned with EtOAc. The organic layer was dried over Na₂SO₄, filtered, and concentrated to provide the title compound (600 mg, 43% isolated yield). LCMS (electrospray): 297 [M+1]. ¹H NMR (CDCl₃) δ 9.19-9.38 (m, 1H) 8.46 (s, 1H) 8.30-8.39 (m, 1H) 7.27-7.44 (m, 2H) 6.60 (d, 1H) 5.42-5.59 (m, 1H) 1.60 (d, 3H).

### Intermediate 10

### N⁶-(5-Cyclopropyl-1H-pyrazol-3-yl)-N²-[(1S)-1-(5-fluoropyridin-2-yl)-ethyl]-3-nitropyridine-2,6-diamine

A mixture of 6-chloro-N-[(1S)-1-(5-fluoropyridin-2-yl)-ethyl]-3-nitropyridin-2-amine (Intermediate 9, 600 mg, 2 mmol), 5-cyclopropyl-1H-pyrazol-3-amine (500 mg, 4 mmol), and DIPEA (0.5 mL, 2.63 mmol) in n-butanol was set to heat in microwave for 30 minutes at 150° C. The residue was purified by flash chromatography (Biotage, 25%→40% EtOAc/hexanes) to provide the title compound (364 mg, 47% isolated yield). LCMS (electrospray): 384 [M+1]. ¹H NMR δ 12.17 (s, 1H) 10.45 (s, 1H) 9.56 (s, 1H) 8.59 (d, 1H) 8.11 (d, 1H) 7.63-7.78 (m, 1H) 7.49 (dd, 1H) 6.13-6.38 (m, 2H) 5.38-5.57 (m, 1H) 1.80-1.95 (m, 1H) 1.57 (d, 3H) 0.96 (d, 2H) 0.71 (s, 2H).

### Intermediate 11

### 5-Ethoxy-1H-pyrazol-3-amine

3-Amino-5-hydroxypyrazole and ethanol were reacted in a procedure analogous to the one described for the synthesis of Intermediate 23, providing the title compound.

¹H NMR (400 MHz, CD₃OD) δ 4.85 (br s, 3H), 4.02 (m, 2H), 1.30 (t, J=8 Hz, 3H)

### Intermediate 12

### N²-(5-Ethoxy-1H-pyrazol-3-yl)-3-fluoro-N⁶-[(1S)-1-(5-fluoropyridin-2-yl)-ethyl]-5-nitropyridine-2,6-diamine

The title compound was prepared using a procedure analogous to the one described for the synthesis of Intermediate 8 using 5,6-difluoro-N-[(1S)-1-(5-fluoropyridin-2-yl)-ethyl]-3-nitropyridin-2-amine (Intermediate 7) and 5-ethoxy-1H-pyrazol-3-amine (Intermediate 11) as the starting materials. The residue was purified by flash chromatography (Biotage, 5%→10% EtOAc in DCM) to provide the title compound as a yellow solid (969 mg, 33% isolated yield). LCMS (electrospray): 406 [M+1]. ¹H NMR (CDCl₃) δ 11.76 (br s, 1H) 11.04 (s, 1H) 8.50 (d, 1H) 8.02 (d, 1H) 7.35-7.53 (m, 2H) 6.49 (d, 1H) 5.44 (s, 1H) 5.31-5.43 (m, 1H) 4.25 (q, 2H) 1.69 (d, 2H) 1.41 (t, 3H).

### Intermediate 13

### 5-Isopropoxy-1H-pyrazol-3-amine

3-Amino-5-hydroxypyrazole and 2-propanol were reacted in a procedure analogous to the one described for the synthesis of Intermediate 23, providing the title compound.

¹H NMR (400 MHz) δ 10.3 (br s, 1H), 4.84 (br s, 2H), 4.65 (s, 1H), 4.52 (m, 1H), 1.20 (m, 6H)

### Intermediate 14

### 3-Fluoro-N⁶-[(1S)-1-(5-fluoropyridin-2-yl)-ethyl]-N²-(5-isopropoxy-1H-pyrazol-3-yl)-5-nitropyridine-2,6-diamine

The title compound was prepared using a procedure analogous to the one described for the synthesis of Intermediate 8 using 5,6-difluoro-N-[(1S)-1-(5-fluoropyridin-2-yl)-ethyl]-3-nitropyridin-2-amine (Intermediate 7), and 5-isopropoxy-1H-pyrazol-3-amine (Intermediate 13) as the starting materials. The residue was purified by flash chromatography (Biotage, 10%→30% ethyl acetate in methylene chloride) to provide the title compound as a yellow solid (670 mg, 41% isolated yield). LCMS (electrospray): 420 [M+1]. ¹H NMR (CDCl₃) δ 11.70 (br s, 1H) 11.04 (s, 1H) 8.50 (s, 1H) 8.03 (d, 1H) 7.34-7.54 (m, 2H) 6.49 (d, 1H) 5.31-5.50 (m, 2H) 4.72-4.88 (m, 1H) 1.69 (d, 3H) 1.38 (dd, 6H).

### Intermediate 15

### 5-Fluoropyrimidine-2-carbonitrile

A 10 ml microwave vial was charged with 2-chloro-5-fluoropyrimidine (2.0 g, 15.09 mmol), Pd₂(dba)₃ (0.549 g, 0.6 mmol), dppf (0.67 g, 1.21 mmol), zinc cyanide (1.15 g, 9.81 mmol), and zinc dust (0.237 mg, 3.62 mmol). The flask was evacuated and backfilled with N₂, and anhydrous DMAc. The vial was mounted onto a Personal Chemistry microwave reactor and heated at 100° C. for 10 hours. The reaction mixture was diluted with EtOAc and then washed with brine three times. The organic layer was obtained and evaporated to dryness. The dried residue was purified by silica gel chromatography (By ISCO Combiflash with gradient EtOAc and hexanes) to afford the title compound as a creamy solid (1.50 g, 80%). GC-MS: 123 (M); ¹H NMR (CDCl₃) δ 8.80 (s, 2H).

### Intermediate 16

### N-(1-(5-Fluoropyrimidin-2-yl)vinyl)acetamide

5-Fluoropyrimidine-2-carbonitrile (Intermediate 15, 1.0 g, 8.1 mmol) in THF (10 ml) was added to a solution of MeMgBr (3.3 ml, 9.75 mmol) in ether drop wise at 0° C. After addition, the reaction was warmed to room temperature, stirred at room temperature for 1 hour and then diluted with DCM (10 ml). Acetic anhydride (1.23 ml, 13.0 mmol) was added in one portion. The reaction was stirred at room temperature for 1 hour and 40° C. for 1 hour. Saturated sodium bicarbonate solution (10 ml) was added and extracted with EtOAc (2×20 ml). The combined organic was dried over sodium sulfate. After removal of solvent, the resulted residue was purified by column chromatography (hexane:EtOAc=2.5:1) to give the title compound as a white solid (0.38 g, 26%). ¹H NMR (400 MHz) 9.34 (s, 1H), 8.95 (s, 2H), 6.25 (s, 1H), 6.03 (s, 1H), 2.11 (s, 3H). LCMS: 182 [M+H]⁺.

### Intermediate 17

### (S)-N-(1-(5-Fluoropyrimidin-2-yl)ethyl)acetamide

N-(1-(5-Fluoropyrimidin-2-yl)vinyl)acetamide (Intermediate 16, 0.10 g, 0.55 mmol) in MeOH (5 ml) under N₂ was added (+)-1,2-bis((2S,5S)-2,5-diethylphospholano)benzene (cyclooctadiene)rhodium(I)trifluoromethanesulfonate (0.04 g, 0.0055 mmol). The solution was transferred to a high pressure bomb and charged 150 psi H₂. The reaction was stirred at room temperature for 4 hours. The solvent was removed and the resulted residue was purified by column chromatography (EtOAc) to give the title compound as a white solid (0.096 g, 95%). ¹H NMR (400 MHz) 8.84 (d, 2H), 8.34 (d, 1H), 5.00 (m, 1H), 1.84 (s, 3H), 1.37 (d, 3H). LCMS: 184 [M+H]⁺. Enantiomeric excess determined by HPLC (Chiralpak IA; 95:5 CO₂/MeOH), >99% ee.

### Intermediate 18

### tert-Butyl [(1S)-1-(5-fluoropyrimidin-2-yl)ethyl]carbamate

(S)-N-(1-(5-Fluoropyrimidin-2-yl)ethyl)acetamide (Intermediate 17, 0.20 g, 1.09 mmol), DMAc (0.027 g, 0.22 mmol) and Boc₂O (0.60 g, 2.73 mmol) in THF (10 ml) was stirred at 50° C. for 40 hours. After cooling to room temperature, lithium hydroxide monohydrate (0.094 g, 2.24 mmol) and water (10 ml) was added. The reaction was stirred at room temperature for 9 hours. Ether (30 ml) was added, organic layer was separated, washed with brine (20 ml) and dried over sodium sulfate. After removal of solvent, the resulted residue was purified by column chromatography (Hex:EtOAc=5:1) to give the title compound as a pale yellow oil (0.21 g, 80%). NMR (400 MHz) 8.84 (s, 2H), 7.24 (d, J=7.6 Hz, 1H), 4.74 (m, 1H), 1.35 (s, 12H). LCMS: 242 [M+H]⁺.

### Intermediate 19

### [(1S)-1-(5-Fluoropyrimidin-2-yl)ethyl]amine hydrochloride

To a solution of tent-butyl [(1S)-1-(5-fluoropyrimidin-2-yl)ethyl]carbamate (Intermediate 18, 0.21 g, 0.87 mmol) in DCM (5 ml) was added HCl (1.3 ml, 5.2 mmol) in dioxane. The reaction was stirred at room temperature for 3 hours. The solvent was removed under vacuum give the title compound as white solid (quantitative). LCMS: 142 [M+H]⁺.

### Intermediate 20

### 5,6-Difluoro-N-[(1S)-1-(5-fluoropyrimidin-2-yl)-ethyl]-3-nitropyridin-2-amine

The title compound was prepared using a procedure analogous to the one described for the synthesis of Intermediate 7 using 2,3,6-trifluoro-5-nitropyridine (Intermediate 6) and [(1S)-1-(5-fluoropyrimidin-2-yl)ethyl]amine hydrochloride (Intermediate 19) as the starting materials.

The residue was purified by flash chromatography (Biotage, 20% EtOAc/hexanes) to provide the title compound as a yellow solid (1.52 g, 81% isolated yield). LCMS (electrospray): 300 [M+1]. ¹H NMR (CDCl₃) δ 8.61 (s, 1H) 7.97-8.16 (m, 1H) 6.92 (d, 1H) 5.46 (t, 1H) 1.65 (d, 2H).

### Intermediate 21

### N²-(5-Cyclopropyl-1H-pyrazol-3-yl)-3-fluoro-N⁶-[(1S)-1-(5-fluoropyrimidin-2-yl)-ethyl]-5-nitropyridine-2,6-diamine

The title compound was prepared using a procedure analogous to the one described for the synthesis of Intermediate 8 using 5,6-difluoro-N-[(1S)-1-(5-fluoropyrimidin-2-yl)-ethyl]-3-nitropyridin-2-amine (Intermediate 20) and 5-cyclopropyl-1H-pyrazol-3-amine as the starting materials. The residue was purified by flash chromatography (Biotage, 50%475% ethyl acetate in hexanes) to provide the title compound as a yellow solid (700 mg, 62% isolated yield). LCMS (electrospray): 403 [M+1]. ¹H NMR (CD₃OD) δ 8.63-8.79 (m, 2H) 8.01 (d, 1H) 6.28 (s, 1H) 5.44-5.62 (m, 1H) 1.85-1.99 (m, 1H) 1.67 (t, 3H) 1.04 (d, 2H) 0.86 (d, 2H).

### Intermediate 22

### 3-Fluoro-N⁶-[(1S)-1-(5-fluoropyrimidin-2-yl)-ethyl]-N²-(5-isopropoxy-1H-pyrazol-3-yl)-5-nitropyridine-2,6-diamine

The title compound was prepared using a procedure analogous to the one described for the synthesis of Intermediate 8 using 5,6-difluoro-N-[(1S)-1-(5-fluoropyrimidin-2-yl)-ethyl]-3-nitropyridin-2-amine (Intermediate 20) and 5-isopropoxy-1H-pyrazol-3-amine (Intermediate 13) as the starting materials. The residue was purified by flash chromatography (Biotage, 15%→30% ethyl acetate in methylene chloride) to provide the title compound as a yellow solid (880 mg, 42% isolated yield). LCMS (electrospray): 421 [M+1]. ¹H NMR (CDCl₃) δ 11.90 (br s, 1H) 11.04 (s, 1H) 8.66 (s, 2H) 8.02 (d, 1H) 6.22 (d, 1H) 5.47-5.60 (m, 1H) 5.44 (s, 1H) 4.68-4.89 (m, 1H) 1.74 (d, 3H) 1.34-1.44 (m, 6H).

### Intermediate 23

### 5-Methoxy-1H-pyrazol-3-amine

To a suspension of 3-amino-5-hydroxypyrazole (50.00 g, 0.50 mol) in CH₂Cl₂ (800 mL) was added triphenylphosphine (155.64 g, 0.59 mol) and the resulting mixture was cooled to 0° C. Diisopropyl azodicarboxylate (117.64 mL, 121 g, 0.59 mol) was added drop-wise over a period of 35 minutes (the temperature of the reaction mixture was kept below 2° C.) to give a dark brown suspension (color differs from time to time). The reaction mixture was then held at 0° C. for 1 hour. An off white precipitation was observed after 30 minutes of the reaction. Methyl alcohol (50 mL, 40 g, 1.25 mol) was then added drop-wise over a period of 30 minutes at 0° C. as the slurry thinned considerably to give a yellow/orange suspension. The reaction mixture was then held at 0° C. for 1 hour. The reaction mixture was warmed slowly to ambient temperature and was then held at ambient temp overnight. The reaction mixture was filtered to remove undissolved solids. The filtrate was dried (MgSO₄) and concentrated under reduced pressure to give yellow-orange oil. Purification by column chromatography (5%→10% MeOH in CH₂Cl₂) afforded the title compound as a waxy solid (5 g). ¹H NMR: δ 4.67 (s, 1H) 3.61 (s, 3H); LCMS: 114 [M+l].

### Intermediate 24

### 3-Fluoro-N⁶-[(1S)-1-(5-fluoropyrimidin-2-yl)ethyl]-N²-(5-methoxy-1H-pyrazol-3-yl)-5-nitropyridine-2,6-diamine

The title compound was prepared using a procedure analogous to the one described for the synthesis of Intermediate 8 using 5,6-difluoro-N-[(1S)-1-(5-fluoropyrimidin-2-yl)-ethyl]-3-nitropyridin-2-amine (Intermediate 20) and 5-methoxy-1H-pyrazol-3-amine (Intermediate 23) as the starting materials. The residue was purified by flash chromatography (Biotage, 50%→75% ethyl acetate in hexanes) to provide the title compound as a yellow solid (586 mg, 30% isolated yield). LCMS (electrospray): 393 [M+1]. ¹H NMR (, CDCl₃) δ 11.92 (br s, 1H) 11.04 (s, 1H) 8.67 (s, 2H) 8.02 (d, 1H) 6.20 (d, 1H) 5.47-5.59 (m, 1H) 5.44 (s, 1H) 3.95 (s, 3H) 1.74 (d, 3H).

### Intermediate 25

### (R)-N-(2-{[tert-Butyl(dimethyl)silyl]oxy}ethylidene)-2-methylpropane-2-sulfinamide

To a solution of (R)-2-methylpropane-2-sulfinamide (2.5 g, 20.6 mmol) and {[tert-butyl(dimethyl)silyl]oxy}acetaldehyde (4.32 ml, 22.7 mmol) in CH₂Cl₂ (30 ml) was added anhydrous CuSO₄ (7.23 g, 45.32 mmol). The reaction mixture was stirred at room temperature for 2 days. The mixture was filtered through Celite®, washed with CH₂Cl₂ and concentrated in vacuo. Purification by column chromatography (Biotage, 0→30% EtOAc in hexanes) provided the title compound. (Tetrahedron Lett. 2001, 42, 2051-54). ¹H NMR (CDCl₃) δ 7.86-8.24 (m, 1H) 4.53 (d, 2H) 1.15-1.23 (m, 9H) 0.90 (s, 9H) 0.08 (s, 6H).

### Intermediate 26

### (R_{S})-N-[(1R)-2-{[tert-Butyl(dimethyl)silyl]oxy}-1-(5-fluoropyridin-2-yl)ethyl]-2-methylpropane-2-sulfinamide*

To a cold solution of 2-bromo-5-fluoropyridine (1.3 g, 7.2 mmol) in Et₂O (8 ml) at −68° C. was added a solution of t-BuLi (1.7 M in pentane, 8.5 ml, 14.4 mmol) with caution. The temperature of the mixture was kept below −65° C. and the mixture was allowed to stir for 15 minutes at −70° C. To a cooled solution (−75° C.) of (R)-N-(2-{[tert-butyl(dimethyl)silyl]oxy}ethylidene)-2-methylpropane-2-sulfinamide (Intermediate 25, 1.0 g, 3.6 mmol) in Et₂O (24 ml) was cannulated a solution of the above lithium compound over 15 minutes. The mixture was allowed to stir at −78° C. for 3 hours whereupon saturated NH₄Cl solution was added. The mixture was diluted with EtOAc and the organic layer was washed with brine and concentrated. Purification by column chromatography (Biotage, 20→40% EtOAc/hexanes) provided the title compound as a solid (higher Rf on TLC, 1.19 g) together with the diastereoisomer (lower Rf on TLC, 166 mg). ¹H NMR (CDCl₃) δ 8.41 (s, 1H) 7.35 (d, 2H) 4.59 (t, 1H) 4.43 (d, 1H) 3.82-4.02 (m, 2H) 1.23 (s, 9H) 0.81 (s, 9H)-0.06 (d, 6H). * Rs indicates that the configuration of sulfur is R

### Intermediate 27

### (2R)-2-Amino-2-(5-fluoropyridin-2-yl)ethanol hydrochloride

To a solution of (Rs)-N-[(1R)-2-{[tert-butyl(dimethyl)silyl]oxy}-1-(5-fluoropyridin-2-yl)ethyl]-2-methylpropane-2-sulfinamide (Intermediate 26, 1.13 g, 3.02 mmol) in MeOH (15 ml) was HCl (4 M in dioxane, 3.02 ml, 12.08 mol) at 0° C. and the mixture was stirred for 15 minutes and then was concentrated. The mixture was triturated from hexanes providing the title compound (575 mg). The product is highly hygroscopic. ¹H NMR δ 8.62 (s, 1H) 8.55 (s, 2H) 7.76-7.93 (m, 1H) 7.65 (dd, 1H) 4.43 (d, 1H) 3.77 (s, 2H).

### Intermediate 28

### (2R)-2-[(5,6-Difluoro-3-nitropyridin-2-yl)-amino]-2-(5-fluoropyridin-2-yl)-ethanol

The title compound was prepared using a procedure analogous to the one described for the synthesis of Intermediate 7 using 2,3,6-trifluoro-5-nitropyridine (Intermediate 6) and (2R)-2-amino-2-(5-fluoropyridin-2-yl)ethanol hydrochloride (Intermediate 27) as the starting materials. The residue was purified by flash chromatography (Biotage, 50% EtOAc/hexanes) to provide the title compound as a yellow solid (1.5 g, 46% isolated yield). LCMS (electrospray): 315 [M+1]. ¹H NMR (CDCl₃) δ 8.42 (s, 1H) 8.00-8.09 (m, 1H) 7.41-7.55 (m, 2H) 6.97 (d, 1H) 5.30-5.37 (m, 1H) 3.93-4.22 (m, 2H) 3.40 (dd, 1H).

### Intermediate 29

### (2R)-2-({5-Fluoro-6-[(5-isopropoxy-1H-pyrazol-3-yl)-amino]-3-nitropyridin-2-yl}amino)-2-(5-fluoropyridin-2-yl)-ethanol

The title compound was prepared using a procedure analogous to the one described for the synthesis of Intermediate 8 using (2R)-2-[(5,6-difluoro-3-nitropyridin-2-yl)-amino]-2-(5-fluoropyridin-2-yl)-ethanol (Intermediate 28) and 5-isopropoxy-1H-pyrazol-3-amine (Intermediate 13) as the starting materials. The residue was purified by flash chromatography (Biotage, 50%→70% ethyl acetate in hexanes) to provide the title compound as a yellow solid (300 mg, 26% isolated yield). LCMS (electrospray): 436 [M+1]. ¹H NMR (CDCl₃) δ 10.90 (s, 1H) 8.48 (s, 1H) 8.03 (d, 1H) 7.43 (t, 2H) 7.19 (d, 1H) 5.36-5.48 (m, 2H) 4.63-4.74 (m, 1H) 3.97-4.17 (m, 2H) 1.31-1.40 (m, 6H).

### Intermediate 30

### (2R)-2-({5-Fluoro-6-[(5-methoxy-1H-pyrazol-3-yl)-amino]-3-nitropyridin-2-yl}-amino)-2-(5-fluoropyridin-2-yl)-ethanol

The title compound was prepared using a procedure analogous to the one described for the synthesis of Intermediate 8 using (2R)-2-[(5,6-difluoro-3-nitropyridin-2-yl)-amino]-2-(5-fluoropyridin-2-yl)ethanol (Intermediate 28) and 5-methoxy-1H-pyrazol-3-amine (Intermediate 23) as the starting materials. The residue was purified by flash chromatography (Biotage, 30%→50% ethyl acetate in methylene chloride) to provide the title compound as a yellow solid (270 mg, 18% isolated yield). LCMS (electrospray): 408 [M+1]. ¹H NMR (CDCl₃) δ 10.90 (s, 1H) 8.49 (s, 1H) 8.03 (d, 1H) 7.44 (d, 2H) 7.17 (s, 1H) 5.38-5.48 (m, 2H) 4.92 (d, 1H) 4.05-4.19 (m, 2H) 3.90 (s, 3H).

### Intermediate 31

### (2R)-2-({6-[(5-Ethoxy-1H-pyrazol-3-yl)amino]-5-fluoro-3-nitropyridin-2-yl}amino)-2-(5-fluoropyridin-2-yl)ethanol

The title compound was prepared using a procedure analogous to the one described for the synthesis of Intermediate 8 using (2R)-2-[(5,6-difluoro-3-nitropyridin-2-yl)amino]-2-(5-fluoropyridin-2-yl)ethanol (Intermediate 28) and 5-ethoxy-1H-pyrazol-3-amine (Intermediate 11) as the starting materials. The residue was purified by flash chromatography (Biotage, 40%→60% ethyl acetate in methylene chloride) to provide the title compound (667 mg, 33% isolated yield). LCMS (electrospray): 422 [M+1]. ¹H NMR (CDCl₃) δ 10.89 (s, 1H) 8.49 (s, 1H) 8.02 (d, 1H) 7.39-7.48 (m, 2H) 7.18 (d, 1H) 5.41-5.50 (m, 1H) 5.39 (s, 1H) 3.95-4.27 (m, 5H) 1.39 (t, 3H).

### Intermediate 32

### 3-Fluoro-N⁶-[(1S)-1-(5-fluoropyridin-2-yl)-ethyl]-N²-(5-methoxy-1H-pyrazol-3-yl)-5-nitropyridine-2,6-diamine

A mixture of 5,6-difluoro-N-[(1S)-1-(5-fluoropyridin-2-yl)ethyl]-3-nitropyridin-2-amine (Intermediate 7, 9.09 g, 30.5 mmol), 5-methoxy-1H-pyrazol-3-amine (Intermediate 23, 4.13 g, 36.6 mmol), and DIPEA (11 mL, 61 mmol) in isopropanol (152 mL) was heated to 75° C. for 16 hours. The residue was triturated from ethyl acetate and hexanes to afford the title compound (5.23 g, 44% isolated yield). LCMS (electrospray): 392 [M+1].

### Intermediate 33

### 3-Fluoro-N⁶-[(1S)-1-(5-fluoropyrimidin-2-yl)ethyl]-N²-(5-methyl-1H-pyrazol-3-yl)-5-nitropyridine-2,6-diamine

To a solution of 5,6-difluoro-N-[(1S)-1-(5-fluoropyrimidin-2-yl)-ethyl]-3-nitropyridin-2-amine (Intermediate 20, 2.81 mmol) in THF (14 ml) was added 5-methyl-1H-pyrazol-3-amine (545 mg, 5.62 mmol) and DIPEA (0.64 ml). The resulting mixture was heated to 55° C. o/n. The resulting mixture was cooled to room temperature and the solvent was removed under reduced pressure to give a colored residue. Purification by column chromatography (Biotage, 50%→75% EtOAc/hexanes) afforded the title compound (470 mg). LCMS: 377 [M+1]. ¹H NMR (MeOD) 8.63-8.78 (m, 2H) 8.01 (d, J=11.30 Hz, 1H) 6.30 (s, 1H) 5.40-5.57 (m, 1H) 2.31 (s, 3H) 1.60-1.80 (m, 3H).

### Intermediate 34

### 3-Fluoro-N⁶-[(1S)-1-(5-fluoropyridin-2-yl)ethyl]-N²-(5-methyl-1H-pyrazol-3-yl)-5-nitropyridine-2,6-diamine

The title compound was prepared using a procedure analogous to the one described for the synthesis of Intermediate 33 using 5,6-difluoro-N-[(1S)-1-(5-fluoropyridin-2-yl)ethyl]-3-nitropyridin-2-amine (Intermediate 7) and 5-methyl-1H-pyrazol-3-amine as starting materials. LCMS: 376 [M+1]. ¹H NMR (CDCl₃) δ 8.48 (d, 1H) 8.03 (d, 1H) 7.28-7.51 (m, 2H) 6.60 (s, 1H) 6.09 (br s, 1H) 5.31-5.47 (m, 1H) 2.34 (s, 3H) 1.67 (d, 3H).

### Intermediate 35

### (2R)-2-({5-Fluoro-6-[(5-methyl-1H-pyrazol-3-yl)amino]-3-nitropyridin-2-yl}amino)-2-(5-fluoropyridin-2-yl)-ethanol

The title compound was prepared using a procedure analogous to the one described for the synthesis of Intermediate 33 using (2R)-2-[(5,6-difluoro-3-nitropyridin-2-yl)amino]-2-(5-fluoropyridin-2-yl)ethanol (Intermediate 28) and 5-methyl-1H-pyrazol-3-amine as starting materials. LCMS: 392 [M+1].

### Intermediate 36

### N-[(3,5-Difluoropyridin-2-yl)methyl]-5,6-difluoro-3-nitropyridin-2-amine

The title compound was prepared using a procedure analogous to the one described for the synthesis of Intermediate 21 using 2,3,6-trifluoro-5-nitropyridine (Intermediate 6) and [(3,5-difluoropyridin-2-yl)methyl]amine as starting materials. LCMS: 303 [M+1].

### Intermediate 37

### N⁶-[(3,5-Difluoropyridin-2-yl)methyl]-3-fluoro-N²-(5-methyl-1H-pyrazol-3-yl)-5-nitropyridine-2,6-diamine

The title compound was prepared using a procedure analogous to the one described for the synthesis of Intermediate 33 using N-[(3,5-difluoropyridin-2-yl)methyl]-5,6-difluoro-3-nitropyridin-2-amine (Intermediate 36) and 5-methyl-1H-pyrazol-3-amine as starting materials. LCMS: 380 [M+1].

### Example 1

### N-(5-Cyclopropyl-1H-pyrazol-3-yl)-6-fluoro-3-[(1S)-1-(5-fluoropyridin-2-yl)-ethyl]-3H-imidazo[4,5-b]-pyridin-5-amine

To a solution of N²-(5-cyclopropyl-1H-pyrazol-3-yl)-3-fluoro-N⁶-[(1S)-1-(5-fluoropyridin-2-yl)ethyl]-5-nitropyridine-2,6-diamine (Intermediate 8, 300 mg, 0.75 mmol) in MeOH-THF (19 mL, 1:1 ratio) was added zinc dust (245 mg, 3.75 mmol) followed by the addition of saturated NH₄Cl_{(aq)} solution (1.9 mL). The resulting mixture was allowed to stir at ambient temperature for 1 hour. Once the reaction showed consumption of starting material, NH₄OAc (2.3 mL) solution was added and this mixture was allowed to stir at ambient temperature for an additional 30 minutes. Ethyl acetate was added and the mixture was filtered through a pad of Celite®. The filtrate was transferred to a separatory funnel and extracted with saturated NaCl_{(aq)} solution. The organic layer was dried over Na₂SO₄, filtered, and concentrated. The residue was dissolved in ethanol (14 mL) followed by the addition of formamidine acetate (166 mg, 1.59 mmol). This mixture was allowed to heat at 95° C. for 15 hours. The reaction mixture was concentrated and purified by flash chromatography (Biotage, 5% MeOH in ethyl acetate) to provide the title compound (85 mg, 30% isolated yield). LCMS (electrospray): 382 [M+1]. ¹H NMR (CD₃OD) δ 8.27-8.37 (m, 13H) 7.62 (d, 1H) 7.49 (d, 2H) 6.15 (s, 1H) 5.17 (d, 1H) 1.89-2.08 (m, 1H) 1.62 (d, 3H) 1.09 (d, 2H) 0.79 (dd, 2H).

### Example 2

### N-(5-Cyclopropyl-1H-pyrazol-3-yl)-3-[(1S)-1-(5-fluoropyridin-2-yl)ethyl]-3H-imidazo[4,5-b]pyridin-5-amine

The title compound was prepared using a procedure analogous to the one described for the synthesis of Example 1 using N⁶-(5-cyclopropyl-1H-pyrazol-3-yl)-N²-[(1S)-1-(5-fluoropyridin-2-yl)-ethyl]-3-nitropyridine-2,6-diamine (Intermediate 10) as the starting material. The residue was purified by flash chromatography (Biotage, 7% methanol in methylene chloride) to provide the title compound (86 mg, 55% isolated yield). LCMS (electrospray): 364 [M+1]. ¹H NMR (400 MHz, CD₃OD) δ 8.51 (d, 1H) 8.30 (s, 1H) 7.79 (d, 1H) 7.49-7.61 (m, 1H) 7.42 (dd, 1H) 6.73 (d, 1H) 5.99 (d, 1H) 5.77 (s, 1H) 1.84-1.96 (m, 1H) 0.91-1.03 (m, 2H) 0.65-0.78 (m, 2H).

### Example 3

### N-(5-Ethoxy-1H-pyrazol-3-yl)-6-fluoro-3-[(1S)-1-(5-fluoropyridin-2-yl)-ethyl]-3H-imidazo[4,5-b]pyridin-5-amine

The title compound was prepared using a procedure analogous to the one described for the synthesis of to Example 1 using N²-(5-ethoxy-1H-pyrazol-3-yl)-3-fluoro-N⁶-[(1S)-1-(5-fluoropyridin-2-yl)-ethyl]-5-nitropyridine-2,6-diamine (Intermediate 12) as the starting material. The residue was purified by flash chromatography (Biotage, 30%→50% acetone in methylene chloride) to provide the title compound (70 mg, 8% isolated yield). LCMS (electrospray): 386 [M+1]. ¹H NMR δ 8.45 (d, 1H) 8.36 (s, 1H) 7.85 (d, 1H) 7.54-7.69 (m, 1H) 7.48 (dd, 1H) 7.36 (d, 1H) 6.01 (s, 1H) 5.18 (t, 1H) 4.07-4.32 (m, 2H) 1.54 (d, 3H) 1.41 (t, 3H).

### Example 4

### 6-Fluoro-3-[(1S)-1-(5-fluoropyridin-2-yl)-ethyl]-N-(5-isopropoxy-1H-pyrazol-3-yl)-3H-imidazo[4,5-b]pyridin-5-amine

The title compound was prepared using a procedure analogous to the one described for the synthesis of Example 1 using 3-fluoro-N⁶-[(1S)-1-(5-fluoropyridin-2-yl)-ethyl]-N²-(5-isopropoxy-1H-pyrazol-3-yl)-5-nitropyridine-2,6-diamine (Intermediate 14) as the starting material. The residue was purified by flash chromatography (Biotage, 8:1:1 of methylene chloride/acetone/ethyl acetate) to provide the title compound (33 mg, 15% isolated yield). LCMS (electrospray): 400 [M+1]. ¹H NMR (CD₃OD) δ 8.38 (s, 1H) 8.33 (s, 1H) 7.63 (d, 1H) 7.49 (d, 2H) 5.96 (s, 1H) 5.15-5.32 (m, 1H) 4.47-4.65 (m, 1H) 1.63 (t, 3H) 1.37-1.49 (m, 6H).

### Example 5

### N-(5-Cyclopropyl-1H-pyrazol-3-yl)-6-fluoro-3-[(1S)-1-(5-fluoropyrimidin-2-yl)-ethyl]-3H-imidazo[4,5-b]pyridin-5-amine

The title compound was prepared using a procedure analogous to the one described for the synthesis of Example 1 using N²-(5-cyclopropyl-1H-pyrazol-3-yl)-3-fluoro-N⁶-[(1S)-1-(5-fluoropyrimidin-2-yl)-ethyl]-5-nitropyridine-2,6-diamine (Intermediate 21) as the starting material. The residue was purified by flash chromatography (Biotage, 65% ethyl acetate in hexanes to 100% ethyl acetate) to provide the title compound (124 mg, 32% isolated yield). LCMS (electrospray): 383 [M+1]. ¹H NMR (CD₃OD) δ 8.65 (s, 2H) 8.33 (s, 1H) 7.58 (d, 1H) 6.40 (s, 1H) 5.31 (d, 1H) 2.00 (s, 1H) 1.65 (d, 3H) 1.11 (d, 2H) 0.86 (t, 2H).

### Example 6

### 6-Fluoro-3-[(1S)-1-(5-fluoropyrimidin-2-yl)-ethyl]-N-(5-isopropoxy-1H-pyrazol-3-yl)-3H-imidazo[4,5-b]pyridin-5-amine

The title compound was prepared using a procedure analogous to the one described for the synthesis of Example 1 using 3-fluoro-N⁶-[(1S)-1-(5-fluoropyrimidin-2-yl)ethyl]-N²-(5-isopropoxy-1H-pyrazol-3-yl)-5-nitropyridine-2,6-diamine (Intermediate 22) as the starting material. The residue was purified by flash chromatography (Biotage, 7:3 of methylene chloride/acetone) followed by a trituration from water/acetonitrile (2:1) to provide the title compound (166 mg, 42% isolated yield). LCMS (electrospray): 401 [M+1]. ¹H NMR (CDCl₃) δ 8.63 (s, 2H) 8.07 (s, 1H) 7.61 (d, 1H) 5.87 (s, 1H) 5.64 (s, 1H) 5.29-5.46 (m, 1H) 4.87 (s, 1H) 1.71 (d, 3H) 1.38-1.48 (m, 6H).

### Example 7

### 6-Fluoro-3-[(1S)-1-(5-fluoropyrimidin-2-yl)-ethyl]-N-(5-methoxy-1H-pyrazol-3-yl)-3H-imidazo[4,5-b]pyridin-5-amine

The title compound was prepared using a procedure analogous to the one described for the synthesis of Example 1 using 3-fluoro-N⁶-[(1S)-1-(5-fluoropyrimidin-2-yl)ethyl]-N²-(5-methoxy-1H-pyrazol-3-yl)-5-nitropyridine-2,6-diamine (Intermediate 24) as the starting material. The residue was purified by flash chromatography (Biotage, 20%→40% acetone in methylene chloride) to provide the title compound (129 mg, 23% isolated yield). LCMS (electrospray): 373 [M+1]. ¹H NMR (CD₃OD) δ 8.66 (s, 2H) 8.33 (s, 1H) 7.61 (d, 1H) 6.25 (s, 1H) 5.35 (t, 1H) 4.05 (s, 3H) 1.66 (d, 3H).

### Example 8

### (2R)-2-{6-Fluoro-5-[(5-isopropoxy-1H-pyrazol-3-yl)-amino]-3H-imidazo[4,5-b]pyridine-3-yl}-2-(5-fluoropyridin-2-yl)-ethanol

The title compound was prepared using a procedure analogous to the one described for the synthesis of Example 1 using (2R)-2-({5-fluoro-6-[(5-isopropoxy-1H-pyrazol-3-yl)-amino]-3-nitropyridin-2-yl}-amino)-2-(5-fluoropyridin-2-yl)-ethanol (Intermediate 29) as the starting material. The residue was purified by flash chromatography (Biotage, 3:1 ethyl acetate/hexanes) to provide the title compound (120 mg, 29% isolated yield). LCMS (electrospray): 416 [M+1]. ¹H NMR (CD₃OD) δ 8.43 (s, 1H) 8.35 (s, 1H) 7.68 (d, 1H) 7.52 (d, 2H) 5.99 (s, 1H) 5.21-5.33 (m, 1H) 4.48-4.63 (m, 1H) 3.91-4.13 (m, 2H) 1.35-1.52 (m, 6H).

### Example 9

### (2R)-2-{6-Fluoro-5-[(5-methoxy-1H-pyrazol-3-yl)-amino]-3H-imidazo[4,5-b]pyridin-3-yl}-2-(5-fluoropyridin-2-yl)-ethanol

The title compound was prepared using a procedure analogous to the one described for the synthesis of Example 1 using (2R)-2-({5-fluoro-6-[(5-methoxy-1H-pyrazol-3-yl)-amino]-3-nitropyridin-2-yl}amino)-2-(5-fluoropyridin-2-yl)ethanol (Intermediate 30) as the starting material. The residue was purified by flash chromatography (Biotage, 90% ethyl acetate in methylene chloride) to provide the title compound (50 mg, 19% isolated yield). LCMS (electrospray): 388 [M+1]. ¹H NMR (CD₃OD) δ 8.44 (s, 1H) 8.35 (s, 1H) 7.67 (d, 1H) 7.53 (d, 2H) 6.06 (s, 1H) 5.28 (t, 1H) 3.89-4.09 (m, 5H).

### Example 10

### (2R)-2-{5-[(5-Ethoxy-1H-pyrazol-3-yl)-amino]-6-fluoro-3H-imidazo[4,5-b]pyridin-3-yl}-2-(5-fluoropyridin-2-yl)-ethanol

The title compound was prepared using a procedure analogous to the one described for the synthesis of Example 1 using (2R)-2-({6-[(5-ethoxy-1H-pyrazol-3-yl)-amino]-5-fluoro-3-nitropyridin-2-yl}-amino)-2-(5-fluoropyridin-2-yl)-ethanol (Intermediate 31) as the starting material. The residue was purified by flash chromatography (silica gel, 90% ethyl acetate in methylene chloride) to provide the title compound (327 mg, 34% isolated yield). LCMS (electrospray): 402 [M+1]. ¹H NMR (CDCl₃) δ 8.46 (s, 1H) 8.11 (s, 1H) 7.62 (d, 1H) 7.32-7.51 (m, 2H) 6.22 (d, 1H) 5.86 (s, 1H) 5.27-5.35 (m, 1H) 4.23 (q, 2H) 4.10 (t, 2H) 1.44 (t, 3H).

### Example 11

### 6-Fluoro-3-[(1S)-1-(5-fluoropyridin-2-yl)-ethyl]-N-(5-methoxy-1H-pyrazol-3-yl)-3H-imidazol-[4,5-b]pyridin-5-amine

To a slurry of 3-fluoro-N⁶-[(1S)-1-(5-fluoropyridin-2-yl)ethyl]-N²-(5-methoxy-1H-pyrazol-3-yl)-5-nitropyridine-2,6-diamine (Intermediate 32, 13.2 g, 8 mmol) in ethanol (150 mL) under N₂ atmosphere was added 10 wt % Palladium/Carbon (700 mg). The reaction was evacuated under vacuum and purged with H₂ (balloon) several times. The reaction was then allowed to stir under H₂ at ambient temperature for 3 hours. As the reaction progresses, the slurry becomes very black and consumption of starting material was monitored by TLC (1:1 of ethyl acetate/hexanes). The reaction was filtered through a pad of Celite® and washed with ethanol (50 mL). The filtrate is then transferred to a round-bottomed flask followed by the addition of formamidine acetate (1.7 g, 16 mmol). The reaction was set to heat at 75° C. for 1 hour. The residue obtained after concentration was then purified by flash chromatography (Biotage, 20%→50% acetone in methylene chloride) to provide the title compound (840 mg, 28% isolated yield). LCMS (electrospray): 372 [M+1]. ¹H NMR δ 11.97 (s, 1H) 10.74-10.96 (m, 1H) 8.93 (d, 1H) 8.48 (s, 1H) 7.96-8.12 (m, 1H) 7.69 (t, 1H) 7.37-7.58 (m, 1H) 5.87 (s, 1H) 5.25-5.57 (m, 1H) 3.83 (s, 3H) 1.59 (d, 3H).

### Example 12

### 6-Fluoro-3-[(1S)-1-(5-fluoropyrimidin-2-yl)ethyl]-N-(5-methy 1-1H-pyrazol-3-yl)-3H-imidazo[4,5-b]pyridin-5-amine

The title compound was prepared using a procedure analogous to the one described for the synthesis of Example 1 using 3-fluoro-N⁶-[(1S)-1-(5-fluoropyrimidin-2-yl)ethyl]-N²-(5-methyl-1H-pyrazol-3-yl)-5-nitropyridine-2,6-diamine (Intermediate 33) as the starting material. LCMS: 357 [M+1]. ¹H NMR (MeOD) δ 8.68 (s, 2H) 8.34 (s, 1H) 7.62 (d, 1H) 6.49 (s, 1H) 5.35 (d, 1H) 2.41 (s, 3H) 1.66 (d, 3H).

### Example 13

### 6-Fluoro-3-[(1S)-1-(5-fluoropyridin-2-yl)ethyl]-N-(5-methyl-1H-pyrazol-3-yl)-3H-imidazo[4,5-b]pyridin-5-amine

The title compound was prepared using a procedure analogous to the one described for the synthesis of Example 1 using 3-fluoro-N⁶-[(1S)-1-(5-fluoropyridin-2-yl)ethyl]-N²-(5-methyl-1,1-pyrazol-3-yl)-5-nitropyridine-2,6-diamine (Intermediate 34) as the starting material. LCMS: 356 [M+1]. ¹H NMR (MeOD) δ 8.43 (s, 1H) 8.32 (s, 1H) 7.61 (d, 1H) 7.50 (d, 2H) 6.23 (s, 1H) 5.17 (q, 1H) 2.37 (s, 3H) 1.61 (d, 3H).

### Example 14

### 6-Fluoro-3-[(1S)-1-(5-fluoropyrimidin-2-yl)ethyl]-5-[(5-methyl-1H-pyrazol-3-yl)amino]-1,3-dihydro-2H-imidazo[4,5-b]pyridin-2-one

To a solution of 3-fluoro-N⁶-[(1S)-1-(5-fluoropyrimidin-2-yl)ethyl]-N²-(5-methyl-1H-pyrazol-3-yl)-5-nitropyridine-2,6-diamine (Intermediate 33, 185 mg, 0.5 mmol) in EtOH (5 ml) were added SnCl₂.2H₂O (553 mg, 2.45 mmol) and tetramethoxymethane (0.652 ml). The resulting solution was heated to 70° C. o/n. The mixture was allowed to cool to room temperature and filtered through Celite® and washed with EtOAc. Evaporation of the volatiles under reduced pressure gave a colored residue that was purified by Gilson (5%→95% MeCN/H₂O) to give the title compound. LCMS: 373 [M+1]. ¹H NMR δ 1.54 (s, 3H) 2.24 (s, 3H) 4.88-5.19 (m, 1H) 5.99 (s, 1H) 6.06 (s, 1H) 7.25 (d, 1H) 8.98 (s, 2H).

### Example 15

### 6-Fluoro-3-[(1S)-1-(5-fluoropyrimidin-2-yl)ethyl]-2-methyl-N-(5-methyl-1H-pyrazol-3-yl)-3H-imidazo[4,5-b]pyridin-5-amine

The title compound was prepared using a procedure analogous to the one described for the synthesis of Example 14 using 3-fluoro-N⁶-[(1S)-1-(5-fluoropyrimidin-2-yl)ethyl]-N²-(5-methyl-1H-pyrazol-3-yl)-5-nitropyridine-2,6-diamine (Intermediate 33), SnCl₂.2H₂O, and triethyl orthoacetate as starting materials. LCMS: 371 [M+1]. ¹H NMR δ 1.56 (d, 3H) 2.33 (s, 3H) 2.54 (s, 3H) 4.92-5.23 (m, 1H) 5.95 (s, 1H) 7.80 (d, 1H) 8.81 (s, 2H).

### Example 16

### (2R)-2-{6-Fluoro-5-[(5-methyl-1H-pyrazol-3-yl)amino]-3H-imidazo[4,5-b]pyridin-3-yl}-2-(5-fluoropyridin-2-yl)ethanol

The title compound was prepared using a procedure analogous to the one described for the synthesis of Example 14 using (2R)-2-({5-fluoro-6-[(5-methyl-1H-pyrazol-3-yl)amino]-3-nitropyridin-2-yl}amino)-2-(5-fluoropyridin-2-yl)ethanol (Intermediate 35), SnCl₂.2H₂O, and triethyl orthoformate as starting materials. LCMS: 372 [M+1]. ¹H NMR δ 2.27 (s, 3H) 3.71-4.02 (m, 2H) 5.04-5.14 (m, 1H) 6.21 (s, 1H) 7.09 (s, 1H) 7.49 (dd, 1H) 7.57-7.68 (m, 1H) 7.87-7.98 (m, 1H) 8.51-8.60 (m, 2H).

### Example 17

### 3-[(3,5-Difluoropyridin-2-yl)methyl]-6-fluoro-N-(5-methyl-1H-pyrazol-3-yl)-3H-imidazo[4,5-b]pyridin-5-amine

The title compound was prepared using a procedure analogous to the one described for the synthesis of Example 14 using N⁶-[(3,5-difluoropyridin-2-yl)methyl]-3-fluoro-N²-(5-methyl-1H-pyrazol-3-yl)-5-nitropyridine-2,6-diamine (Intermediate 37), SnCl₂.2H₂O, and triethylorthoformate as starting materials. LCMS: 360 [M+1]. ¹H NMR δ 2.32 (s, 3H) 4.75 (s, 2H) 6.33-6.51 (m, 1H) 7.86 (d, 2H) 7.90-8.00 (m, 1H) 8.41-8.47 (m, 2H).

### Example 18

### 3-[(3,5-Difluoropyridin-2-yl)methyl]-6-fluoro-2-methyl-N-(5-methyl-1H-pyrazol-3-yl)-3H-imidazo[4,5-b]pyridin-5-amine

The title compound was prepared using a procedure analogous to the one described for the synthesis of Example 14 using N⁶-[(3,5-difluoropyridin-2-yl)methyl]-3-fluoro-N²-(5-methyl-1H-pyrazol-3-yl)-5-nitropyridine-2,6-diamine (Intermediate 37), SnCl₂.2H₂O, and triethylorthoacetate as starting materials. LCMS: 374 [M+1]. ¹H NMR δ 2.32 (s, 3H) 3.29-3.36 (m, 3H) 4.63 (d, 2H) 6.09 (s, 1H) 7.15 (s, 1H) 7.69 (s, 1H) 7.84-7.94 (m, 1H) 8.40 (s, 1H) 12.69 (s, 1H).

What is claimed is:

1. A compound of Formula (I): 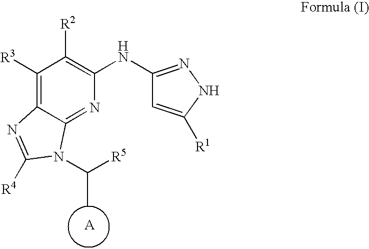
or a pharmaceutically acceptable salt thereof, wherein
Ring A is selected from heterocyclyl, wherein said heterocyclyl is optionally substituted with one or more R⁶;
R¹ is selected from H, —CN, C₁₋₆alkyl, C₂₋₆alkenyl, C₂₋₆alkynyl, 3- to 5-membered carbocyclyl, 5-membered heterocyclyl, —OR^{1a}, —SR^{1a}, —N(R^{1a})₂, —N(R^{1a})C(O)R^{1b}, —N(R^{1a})N(R^{1a})₂, —NO₂, —C(O)H, —C(O)R^{1b}, —C(O)₂R^{1a}, —C(O)N(R^{1a})₂, —OC(O)N(R^{1a})₂, —N(R^{1a})C(O)₂R^{1a}, —N(R^{1a})C(O)N(R^{la})₂, —OC(O)R^{1b}, —S(O)R^{1b}, —S(O)₂R^{1b}, —S(O)₂N(R^{ia})₂, —N(R^{1a})S(O)₂R^{1b}, —C(R^{1a})═N(R^{1a}), and —C(R^{1a})═N(OR^{1a}), wherein said C₁₋₆alkyl, C₂₋₆alkenyl, C₂₋₆alkynyl, 3- to 5-membered carbocyclyl, and 5-membered heterocyclyl are optionally substituted with one or more R¹⁰;
R^{1a} in each occurrence is independently selected from H, C₁₋₆alkyl, 3- to 5-membered carbocyclyl, and 5-membered heterocyclyl, wherein said C₁₋₆alkyl, 3- to 5-membered carbocyclyl, and 5-membered heterocyclyl in each occurrence are optionally and independently substituted with one or more R¹⁰;
R^{1b} in each occurrence is independently selected from C₁₋₆alkyl, C₂₋₆alkenyl, C₂₋₆alkynyl, 3- to 5-membered carbocyclyl, and 5-membered heterocyclyl wherein said C₁₋₆alkyl, C₂₋₆alkenyl, C₂₋₆alkynyl, 3- to 5-membered carbocyclyl, and 5-membered heterocyclyl in each occurrence are optionally and independently substituted with one or more R¹⁰;
R² is selected from H, halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —N(R^{2a})N(R^{2a})₂, —NO₂, —C(O)H, —C(O)R^{2b}, —C(O)₂R^{2a}, —C(O)N(R^{2a})₂, —OC(O)N(R^{2a})₂, —N(R^{2a})C(O)₂R^{2a}, —N(R^{2a})C(O)N(R^{2a})₂, —OC(O)R^{2b}, —S(O)R^{2b}, —S(O)₂R^{2b}, —S(O)₂N(R^{2a})₂, —N(R^{2a})S(O)₂R^{2b}, —C(R^{2a})═N(R^{2a}), and —C(R^{2a})═N(OR^{2a}), wherein said C₁₋₆alkyl, C₂₋₆alkenyl, C₂₋₆alkynyl, carbocyclyl, and heterocyclyl are optionally substituted with one or more R²⁰;
R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R²⁰;
R^{2b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R²⁰;
R³ is selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —N(R^{3a})N(R^{3a})₂, —NO₂, —C(O)H, —C(O)R^{3b}, —C(O)₂R^{3a}, —C(O)N(R^{3a})₂, —OC(O)N(R^{3a})₂, —N(R^{3a})C(O)₂R^{3a}, —N(R^{3a})C(O)N(R^{3a})₂, —OC(O)R^{3b}, —S(O)R^{3b}, —S(O)₂R^{3b}, —S(O)₂N(R^{3a})₂, —N(R^{3a})S(O)₂R^{3b}, —C(R^{3a})═N(R^{3a}), and —C(R^{3a})═N(OR^{3a}), wherein said C₁₋₆alkyl, C₂₋₆alkenyl, C₂₋₆alkynyl, carbocyclyl, and heterocyclyl are optionally substituted with one or more R³⁰;
R^{3a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R³⁰;
R^{3b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R³⁰;
R⁴ is selected from H, halo, —CN, C₁₋₆alkyl, C₂₋₆alkenyl, C₂₋₆alkynyl, carbocyclyl, heterocyclyl, —OR^{4a}, —SR^{4a}, —N(R^{4a})₂, —N(R^{4a})C(O)R^{4b}, —N(R^{4a})N(R^{4a})₂, —NO₂, —C(O)H, —C(O)R^{4b}, —C(O)₂R^{4a}, —C(O)N(R^{4a})₂, —OC(O)N(R^{4a})₂, —N(R^{4a})C(O)₂R^{4a}, —N(R^{4a})C(O)N(R^{4a})₂, —OC(O)R^{4b}, —S(O)R^{4b}, —S(O)₂R^{4b}, —S(O)₂N(R^{4a})₂, —N(R^{4a})S(O)₂R^{4b}, —C(R^{4a})═N(R^{4a}), and —C(R^{4a})═N(OR^{4a}), wherein said C₁₋₆alkyl, C₂₋₆alkenyl, C₂₋₆alkynyl, carbocyclyl, and heterocyclyl are optionally substituted with one or more R⁴⁰;
R^{4a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R⁴⁰;
R^{4b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R⁴⁰;
R⁵ is selected from H, —CN, C₁₋₆alkyl, C₂₋₆alkenyl, C₂₋₆alkynyl, carbocyclyl, heterocyclyl, —N(R^{5a})C(O)R^{5b}, —N(R^{5a})N(R^{5a})₂, —NO₂, —C(O)H, —C(O)R^{5b}, —C(O)₂R^{5a}, —C(O)N(R^{5a})₂, —OC(O)N(R⁵¹₂, —N(R^{5a})C(O)₂R^{5a}, —N(R^{5a})C(O)N(R^{5a})₂, —OC(O)R^{5b}, —S(O)R^{5b}, —S(O)₂R^{5b}, —S(O)₂N(R^{5a})₂, —N(R^{5a})S(O)₂R^{5b}, —C(R^{5a})═N(R^{5a}), and —C(R^{5a})═N(OR^{5a}), wherein said C₁₋₆alkyl, C₂₋₆alkenyl, C₂₋₆alkynyl, carbocyclyl, and heterocyclyl are optionally substituted with one or more R⁵⁰;
R^{5a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R⁵⁰;
R^{5b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R⁵⁰;
R⁶ in each occurrence is independently selected from halo, —ON, C₁₋₆alkyl, C₂₋₆alkenyl, C₂₋₆alkynyl, carbocyclyl, heterocyclyl, —OR^{6a}, —SR^{6a}, —N(R^{6a})₂, —N(R^{6a})C(O)R^{6b}, —N(R^{6a})N(R^{6a})₂, —NO₂, —C(O)H, —C(O)R^{6b}, —C(O)₂R^{6a}, —C(O)N(R^{6a})₂, —OC(O)N(R⁶¹₂, —N(R^{6a})C(O)₂R^{6a}, —N(R^{6a})C(O)N(R^{6a})₂, —OC(O)R^{6b}, —S(O)R^{6b}, —S(O)₂R^{6b}, —S(O)₂N(R^{6a})₂, —N(R^{6a})S(O)₂R^{6b}, —C(R^{6a})═N(R^{6a}), and —C(R^{6a})═N(OR^{6a}), wherein said C₁₋₆alkyl, C₂₋₆alkenyl, C₂₋₆alkynyl, carbocyclyl, and heterocyclyl are optionally substituted with one or more R⁶⁰;
R^{6a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R⁶⁰;
R^{6b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R⁶⁰;
R¹⁰ in each occurrence is independently selected from halo, —ON, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})C(O)R^{10b}, —N(R^{10a})N(R^{10a})₂, —NO₂, —C(O)H, —C(O)R^{10b}, —C(O)₂R^{10a}, —C(O)N(R^{10a})₂, —OC(O)N(R^{10a})₂, —N(R^{10a})C(O)₂R^{10a}, —N(R^{10a})C(O)N(R^{10a})₂, —OC(O)R^{10b}, —S(O)R^{10b}, —S(O)₂R^{10b}, —S(O)₂N(R^{10a})₂, —N(R^{10a})S(O)₂R^{10b}, —C(R^{10a})═N(R^{10a}), and —C(R^{10a})═N(OR^{10a}), wherein said C₁₋₆alkyl, C₂₋₆alkenyl, C₂₋₆alkynyl, carbocyclyl, and heterocyclyl in each occurrence are optionally and independently substituted with one or more R^{a};
R^{10a} in each occurrence is independently selected from H and C₁₋₆alkyl, wherein said C₁₋₆alkyl in each occurrence is optionally and independently substituted with one or more R^{a};
R^{10b} in each occurrence is independently selected from C₁₋₆alkyl, C₂₋₆alkenyl, and C₂₋₆alkynyl, wherein said C₁₋₆alkyl, C₂₋₆alkenyl, and C₂₋₆alkynyl in each occurrence are optionally and independently substituted with one or more R^{a};
R²⁰ in each occurrence is independently selected from halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, —N(R^{20a})C(O)R^{20b}, —N(R^{20a})N(R^{20a})₂, —NO₂, —C(O)H, —C(O)R^{20b}, —C(O)₂R^{20a}, —C(O)N(R^{20a})₂, —OC(O)N(R^{20a})₂, —N(R^{20a})C(O)₂R^{20a}, —N(R^{20a})C(O)N(R^{20a})₂, —OC(O)R^{20b}, —S(O)R^{20b}, —S(O)₂R^{20b}, —S(O)₂N(R^{20a})₂, —N(R^{20a})S(O)₂R^{20b}, —C(R^{20a})═N(R^{20a}), and —C(R^{20a})═N(OR^{20a}), wherein said C₁₋₆alkyl, C₂₋₆alkenyl, C₂₋₆alkynyl, carbocyclyl, and heterocyclyl in each occurrence are optionally and independently substituted with one or more R^{b};
R^{20a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R^{b};
R^{20b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R^{b};
R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})C(O)R^{30b}, —N(R^{30a})N(R^{30a})₂, —NO₂, —C(O)H, —C(O)R^{30b}, —C(O)₂R^{30a}, —C(O)N(R^{30a})₂, —OC(O)N(R^{30a})₂, —N(R^{30a})C(O)₂R^{30a}, —N(R^{30a})C(O)N(R^{30a})₂, —OC(O)R^{30b}, —S(O)R^{30b}, —S(O)₂R^{30b}, —S(O)₂N(R^{30a})₂, —N(R^{30a})S(O)₂R^{30b}, —C(R^{30a})═N(R^{30a}), and —C(R^{30a})═N(OR^{30a}), wherein said C₁₋₆alkyl, C₂₋₆alkenyl, C₂₋₆alkynyl, carbocyclyl, and heterocyclyl in each occurrence are optionally and independently substituted with one or more R^{c};
R^{30a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R^{c};
R^{30b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R^{c};
R⁴⁰ in each occurrence is independently selected from halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})C(O)R^{40b}, —N(R^{40a})N(R^{40a})₂, —NO₂, —C(O)H, —C(O)R^{40b}, —C(O)₂R^{40a}, —C(O)N(R^{40a})₂, —OC(O)N(R^{40a})₂, —N(R^{40a})C(O)₂R^{40a}, —N(R^{40a})C(O)N(R^{40a})₂, —OC(O)R^{40b}, —S(O)R^{40b}, —S(O)₂R^{40b}, —S(O)₂N(R^{40a})₂, —N(R^{40a})S(O)₂R^{40b}, —C(R^{40a})═N(R^{40a}), and —C(R^{40a})═N(OR^{40a}), wherein said C₁₋₆alkyl, C₂₋₆alkenyl, C₂₋₆alkynyl, carbocyclyl, and heterocyclyl in each occurrence are optionally and independently substituted with one or more R^{d};
R^{40a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R^{d};
R^{40b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R^{d};
R⁵⁰ in each occurrence is independently selected from halo, —CN, C₁₋₆alkyl, C₂₋₆alkenyl, C₂₋₆alkynyl, carbocyclyl, heterocyclyl, —OR^{50a}, —SR^{50a}, —N(R^{50a})₂, —N(R^{50a})C(O)R^{50b}, —N(R^{50a})N(R^{50a})₂, —NO₂, —C(O)H, —C(O)R^{50b}, —C(O)₂R^{50a}, —C(O)N(R^{50a})₂, —OC(O)N(R^{50a})₂, —N(R^{50a})C(O)₂R^{50a}, —N(R^{50a})C(O)N(R^{50a})₂, —OC(O)R^{50b}, —S(O)R^{50b}, —S(O)₂R^{50b}, —S(O)₂N(R^{50a})₂, —N(R^{50a})S(O)₂R^{50b}, —C(R^{50a})═N(R^{50a}), and —C(R^{50a})═N(OR^{50a}), wherein said C₁₋₆alkyl, C₂₋₆alkenyl, C₂₋₆alkynyl, carbocyclyl, and heterocyclyl in each occurrence are optionally and independently substituted with one or more R^{e};
R^{50a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R^{e};
R^{50b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R^{e};
R⁶⁰ in each occurrence is independently selected from halo, —CN, C₁₋₆alkyl, C₂₋₆alkenyl, C₂₋₆alkynyl, carbocyclyl, heterocyclyl, —OR^{60a}, —SR^{60a}, —N(R^{60a})₂, —N(R^{60a})C(O)R^{60b}, —N(R^{60a})N(R^{60a})₂, —NO₂, —C(O)H, —C(O)R^{60b}, —C(O)₂R^{60a}, —C(O)N(R^{60a})₂, —OC(O)N(R^{60a})₂, —N(R^{60a})C(O)₂R^{60a}, —N(R^{60a})C(O)N(R^{60a})₂, —OC(O)R^{60b}, —S(O)R^{60b}, —S(O)₂R^{60b}, —S(O)₂N(R^{60a})₂, —N(R^{60a})S(O)₂R^{60b}, —C(R^{60a})═N(R^{60a}), and —C(R^{60a})═N(OR^{60a}), wherein said C₁₋₆alkyl, C₂₋₆alkenyl, C₂₋₆alkynyl, carbocyclyl, and heterocyclyl in each occurrence are optionally and independently substituted with one or more IV;
R^{60a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more IV;
R^{60b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more IV;
R^{a}, R^{b}, R^{c}, R^{d}, R^{e} and R^{f} in each occurrence are independently selected from halo, —CN, C₁₋₆alkyl, C₂₋₆alkenyl, C₂₋₆alkynyl, carbocyclyl, heterocyclyl, —OR^{m}, —SR^{m}, —N(R^{m})₂, —N(R^{m})C(O)Rⁿ, —N(R^{m})N(R^{m})₂, —NO₂, —C(O)H, —C(O)Rⁿ, —C(O)₂R^{m}, —C(O)N(R^{m})₂, —OC(O)N(R^{m})₂, —N(R^{m})C(O)₂R^{m}, —N(R^{m})C(O)N(R^{m})₂, —OC(O)Rⁿ, —S(O)Rⁿ, —S(O)₂Rⁿ, —S(O)₂N(R^{m})₂, —N(R^{m})S(O)₂Rⁿ, —C(R^{m})═N(R^{m}), and —C(R^{m})═N(OR^{m});
R^{m} in each occurrence is independently selected from H and C₁₋₆alkyl; and
Rⁿ is C₁₋₆alkyl.

2. A compound of Formula (I), or a pharmaceutically acceptable salt thereof, as claimed in claim 1, wherein
Ring A is selected from 6-membered heterocyclyl, wherein said 6-membered heterocyclyl are optionally substituted with one or more R⁶;
R⁶ in each occurrence is independently selected from halo, —CN, C₁₋₆alkyl, C₂₋₆alkenyl, C₂₋₆alkynyl, carbocyclyl, and heterocyclyl, —OR^{6a}, —SR^{6a}, —N(R^{6a})₂, —N(R^{6a})C(O)R^{6b}, —NO₂, —C(O)R^{6b}, —C(O)₂R^{6a}—C(O)N(R^{6a})₂, —OC(O)R^{6a}, —N(R^{6a})C(O)N(R^{6a})₂, —S(O)R^{6b}, —S(O)₂R^{6b}, —S(O)₂N(R^{6a})₂, and —N(R^{6a})S(O)₂R^{6b}, wherein said C₁₋₆alkyl, C₂₋₆alkenyl, C₂₋₆alkynyl, carbocyclyl, and heterocyclyl are optionally substituted with one or more R⁶⁰;
R^{6a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R⁶⁰;
R^{6b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R⁶⁰;
R⁶⁰ in each occurrence is independently selected from halo, —CN, C₁₋₆alkyl, C₂₋₆alkenyl, C₂₋₆alkynyl, carbocyclyl, and heterocyclyl, —OR^{60a}, —SR^{60a}, —N(R^{60a})₂, —N(R^{60a})C(O)R^{60b}, —NO₂, —C(O)H, —C(O)R^{60b}, —C(O)₂R^{60a}, —C(O)N(R^{60a})₂, —OC(O)R^{60a}, —N(R^{60a})C(O)N(R^{60a})₂, —S(O)R^{60b}, —S(O)₂R^{60b}, —S(O)₂N(R^{60a})₂, and —N(R^{60a})S(O)₂R^{60b};
R^{60a} in each occurrence is independently selected from H, C₁₋₆alkyl, carbocyclyl, and heterocyclyl; and
R^{60b} in each occurrence is independently selected from C₁₋₆alkyl, C₂₋₆alkenyl, C₂₋₆alkynyl, carbocyclyl, and heterocyclyl.

3. A compound of Formula (I), or a pharmaceutically acceptable salt thereof, as claimed in claim 1, wherein
R¹ is selected from —CN, C₁₋₆alkyl, C₂₋₆alkenyl, C₂₋₆alkynyl, 3- to 5-membered carbocyclyl, 5-membered heterocyclyl, —OR^{1a}, —SR^{1a}, —N(R^{1a})₂, —N(R^{1a})C(O)R^{1b}, —NO₂, —C(O)H, —C(O)R^{1b}, —C(O)₂R^{1a}, —C(O)N(R^{1a})₂, —OC(O)R^{1b}, —N(R^{1a})C(O)N(R^{1a})₂, —S(O)R^{1b}, —S(O)₂R^{1b}, —S(O)₂N(R^{1a})₂, and —N(R^{1a})S(O)₂R^{1b}, wherein said C₁₋₆alkyl, C₂₋₆alkenyl, C₂₋₆alkynyl, 3- to 5-membered carbocyclyl, and 5-membered heterocyclyl are optionally substituted with one or more R¹⁰;
R^{1a} in each occurrence is independently selected from H, C₁₋₆alkyl, 3- to 5-membered carbocyclyl, and 5-membered heterocyclyl, wherein said C₁₋₆alkyl, 3- to 5-membered carbocyclyl, and 5-membered heterocyclyl in each occurrence are optionally and independently substituted with one or more R¹⁰;
R^{1b} in each occurrence is independently selected from C₁₋₆alkyl, C₂₋₆alkenyl, C₂₋₆alkynyl, 3- to 5-membered carbocyclyl, and 5-membered heterocyclyl wherein said C₁₋₆alkyl, C₂₋₆alkenyl, C₂₋₆alkynyl, 3- to 5-membered carbocyclyl, and 5-membered heterocyclyl in each occurrence are optionally and independently substituted with one or more R¹⁰;
R¹⁰ in each occurrence is independently selected from halo, —CN, C₁₋₆alkyl, C₂₋₆alkenyl, C₂₋₆alkynyl —OR^{10a}, —SR^{10a}, —N(R^{10a})₂, —N(R^{10a})C(O)R^{10b}, —NO₂, —C(O)H, —C(O)R^{10b}, —C(O)₂R^{10a}, —C(O)N(R^{10a})₂, —OC(O)R^{10b}, —N(R^{10a})C(O)N(R^{10a})₂, —S(O)R^{10b}, —S(O)₂R^{10b}, —S(O)₂N(R^{10a})₂, and —N(R^{10a})S(O)₂R^{10b};
R^{10a} in each occurrence is independently selected from H, C₁₋₆alkyl, carbocyclyl, and heterocyclyl; and
R^{10b} in each occurrence is independently selected from C₁₋₆alkyl, C₂₋₆alkenyl, C₂₋₆alkynyl, carbocyclyl, and heterocyclyl.

4. A compound of Formula (I), or a pharmaceutically acceptable salt thereof, as claimed in claim 1, wherein
R² is selected from H, halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —NO₂, —C(O)H, —C(O)R^{2b}, —C(O)₂R^{2a}, —C(O)N(R^{2a})₂, —OC(O)R^{2a}, —N(R^{2a})C(O)N(R^{2a})₂, —S(O)R^{2b}, —S(O)₂R^{2b}, —S(O)₂N(R^{2a})₂, and —N(R^{2a})S(O)₂R^{2b}, wherein said C₁₋₆alkyl, C₂₋₆alkenyl, and C₂₋₆alkynyl, carbocyclyl, and heterocyclyl are optionally substituted with one or more R²⁰;
R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R²⁰;
R^{2b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R²⁰;
R²⁰ in each occurrence is independently selected from halo, —CN, —OR^{20a}, —SR^{20a}, —N(R^{20a})₂, —N(R^{20a})C(O)R^{20b}, —NO₂, —C(O)H, —C(O)R^{20b}, —C(O)₂R^{20a}, —C(O)N(R^{20a})₂, —OC(O)R^{20a}, —N(R^{20a})C(O)N(R^{20a})₂, —S(O)R^{20b}, —S(O)₂R^{20b}, —S(O)₂N(R^{20a})₂, and —N(R^{20a})S(O)₂R^{20b};
R^{20a} in each occurrence is independently selected from H, C₁₋₆alkyl, carbocyclyl, and heterocyclyl; and
R^{20b} in each occurrence is independently selected from C₁₋₆alkyl, C₂₋₆alkenyl, C₂₋₆alkynyl, carbocyclyl, and heterocyclyl.

5. A compound of Formula (I), or a pharmaceutically acceptable salt thereof, as claimed in claim 1, wherein
R³ is selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —NO₂, —C(O)H, —C(O)R^{3b}, —C(O)₂R^{3a}, —C(O)N(R^{3a})₂, —OC(O)R^{2a}, —N(R^{3a})C(O)N(R^{3a})₂, —S(O)R^{3b}, —S(O)₂R^{3b}, —S(O)₂N(R^{3a})₂, and —N(R^{3a})S(O)₂R^{3b}, wherein said C₁₋₆alkyl, C₂₋₆alkenyl, and C₂₋₆alkynyl, carbocyclyl, and heterocyclyl are optionally substituted with one or more R³⁰;
R^{3a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R³⁰;
R^{3b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R³⁰;
R³⁰ in each occurrence is independently selected from halo, —CN, —OR^{30a}, —SR^{30a}, —N(R^{30a})₂, —N(R^{30a})C(O)R^{30b}, —NO₂, —C(O)H, —C(O)R^{30b}, —C(O)₂R^{30a}, —C(O)N(R^{30a})₂, —OC(O)R^{30a}, —N(R^{30a})C(O)N(R^{30a})₂, —S(O)R^{30b}, —S(O)₂R^{30b}, —S(O)₂N(R^{30a})₂, and —N(R^{30a})S(O)₂R^{30b};
R^{30a} in each occurrence is independently selected from H, C₁₋₆alkyl, carbocyclyl, and heterocyclyl; and
R^{30b} in each occurrence is independently selected from C₁₋₆alkyl, C₂₋₆alkenyl, C₂₋₆alkynyl, carbocyclyl, and heterocyclyl.

6. A compound of Formula (I), or a pharmaceutically acceptable salt thereof, as claimed in claim 1, wherein
R⁴ is selected from H, C₁₋₆alkyl, and —OR^{4a}; and
R^{4a} is selected from H and C₁₋₆alkyl.

7. A compound of Formula (I), or a pharmaceutically acceptable salt thereof, as claimed in claim 1, wherein
R⁵ is selected from H, —CN, C₁₋₆alkyl, C₂₋₆alkenyl, and C₂₋₆alkynyl, —N(R^{5a})C(O)R^{5b}, —NO₂, —C(O)H, —C(O)R^{5b}, —C(O)₂R^{5a}, —C(O)N(R^{5a})₂, —OC(O)N(R^{5a})₂, —N(R^{5a})C(O)₂R^{5a}, —N(R^{5a})C(O)N(R^{5a})₂, —OC(O)R^{5b}, —S(O)R^{5b}, —S(O)₂R^{5b}, —S(O)₂N(R^{5a})₂, and —N(R^{5a})S(O)₂R^{5b}, wherein said C₁₋₆alkyl, C₂₋₆alkenyl, and C₂₋₆alkynyl are optionally substituted with one or more R⁵⁰;
R^{5a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted with one or more R⁵⁰;
R^{5b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted with one or more R⁵⁰;
R⁵⁰ in each occurrence is independently selected from halo, —CN, carbocyclyl, heterocyclyl, —OR^{50a}, —SR^{50a}, —N(R^{50a})₂, —N(R^{50a})C(O)R^{50b}, —NO₂, —C(O)H, —C(O)R^{50b}, —C(O)₂R^{50a}, —C(O)N(R^{50a})₂, —OC(O)R^{50a}, —N(R^{50a})C(O)N(R^{50a})₂, —S(O)R^{50b}, —S(O)₂R^{50b}, —S(O)₂N(R^{50a})₂, and —N(R^{50a})S(O)₂R^{50b};
R^{50a} in each occurrence is independently selected from H, C₁₋₆alkyl, carbocyclyl, and heterocyclyl; and
R^{50b} in each occurrence is independently selected from C₁₋₆alkyl, C₂₋₆alkenyl, C₂₋₆alkynyl, carbocyclyl, and heterocyclyl.

8. A compound of Formula (I): 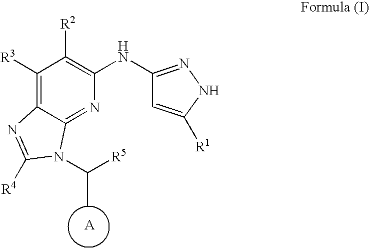
or a pharmaceutically acceptable salt thereof, wherein
Ring A is selected from 5-fluoropyridin-2-yl, 3,5-difluoropyridin-2-yl, and 5-fluoropyrimidin-2-yl;
R¹ is selected from methyl, cyclopropyl, methoxy, ethoxy, and isopropoxy;
R² is selected from H and fluoro;
R³ is H;
R⁴ is selected from H, methyl, and hydroxy; and
R⁵ is selected from H, methyl, and hydroxymethyl.

9. A compound of Formula (I), or a pharmaceutically acceptable salt thereof, as claimed in claim 1, for use as a medicament.

10. The use of a compound of Formula (I), or a pharmaceutically acceptable salt thereof, as claimed in claim 1, in the manufacture of a medicament for the treatment of cancer.

11. A method for treating cancer in a warm-blooded animal such as man, said method comprising administering to said animal an effective amount of a compound of Formula (I), or a pharmaceutically acceptable salt thereof, as claimed in claim 1.

12. A compound of Formula (I), or a pharmaceutically acceptable salt thereof, as claimed in claim 1, for use in the production of a JAK inhibitory effect in a warm-blooded animal such as man.

13. A pharmaceutical composition comprising a compound of Formula (I), or a pharmaceutically acceptable salt thereof, as claimed in claim 1, and at least one pharmaceutically acceptable carrier, diluent, or excipient.

14. A process for preparing a compound of Formula (I) as claimed in claim 1, wherein said process is selected from:
1) Process A—reacting a compound of Formula (A): 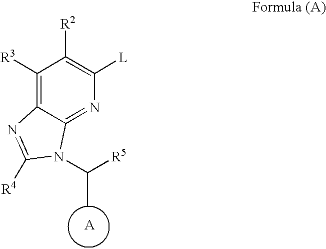
with a compound of Formula (B): 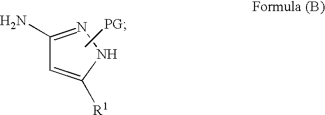
2) Process B—reacting a compound of Formula (C): 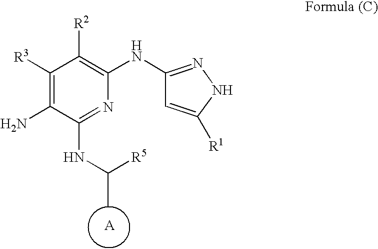
with a compound of Formula (D): 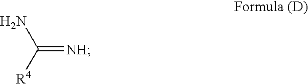
3) Process C—reacting a compound of Formula (E): 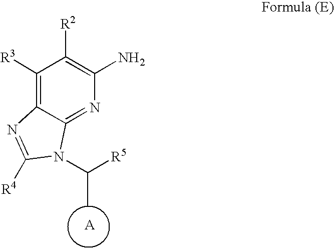
with a compound of Formula (F): 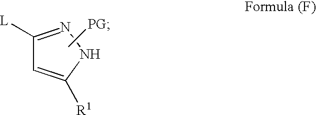
and
4) Process D reacting a compound of Formula (G): 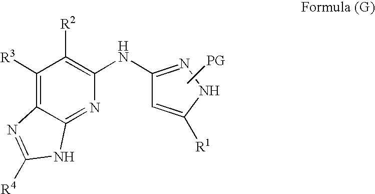
with a compound of Formula (H): 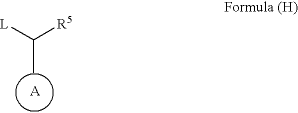
and thereafter if appropriate:
i. converting a compound of Formula (I) into another compound of Formula (I);
ii. removing any protecting groups; and/or
iii. forming a pharmaceutically acceptable salt,
wherein
L in each occurrence is the same or different, and is a leaving group; and
PG in each occurrence is the same or different, and is a protecting group.
